(19) United States
(12) Patent Application Publication
(10) Pub. No.: US 2026/0057960 A1
(43) Pub. Date: Feb. 26, 2026

(54) SYSTEMS AND METHODS FOR PREDICTING RESPONSE TO ANTI-TNF THERAPIES

(71) Applicant: Scipher Medicine Corporation, Waltham, MA (US)

(72) Inventors: Susan GHIASSIAN, Coquitlam (CA); Marc SANTOLINI, Waltham, MA (US); Nancy SCHOENBRUNNER, Charlestown, MA (US); Keith J. JOHNSON, Wayland, MA (US); Ivan VOITALOV, Brighton, MA (US)

(21) Appl. No.: 19/083,334

(22) Filed: Mar. 18, 2025

Related U.S. Application Data
(63) Continuation of application No. 18/793,615, filed on Aug. 2, 2024, now abandoned.
Application No. 18/793,615 is a continuation of application No. PCT/US23/62003, filed on Feb. 3, 2023.
(60) Provisional application No. 63/306,770, filed on Feb. 4, 2022.
(60) Provisional application No. 63/340,360, filed on May 10, 2022.

(51) Int. Cl. G16B 5/20; G16B 25/10; G16B 40/20

(52) U.S. Cl. CPC G16B 5/20; G16B 25/10; G16B 40/20

(57) ABSTRACT

Disclosed herein are methods and systems for administering therapy to subjects who have been determined to display or not display a gene expression response signature established to distinguish between responsive and non-responsive prior subjects who have received the therapy. The subject and prior subjects may suffer from a disease, disorder, or condition for which it is desired to predict whether the subject will respond to the therapy. In an aspect, the disease, disorder, or condition may be an autoimmune disorder such as ulcerative colitis. The subject may be administered an anti-TNF therapy or an alternative to anti-TNF therapy based upon predictions provided by methods and systems described herein.

### CROSS-REFERENCE TO RELATED APPLICATIONS

The present application is a continuation U.S. application Ser. No. 18/793,615, filed Aug. 2, 2024, which is a continuation of International Application No. PCT/US2023/062003, filed Feb. 3, 2023, which claims priority to U.S. Provisional Application No. 63/306,770, filed Feb. 4, 2022, and U.S. Provisional Application No. 63/340,360, filed May 10, 2022, each of which is incorporated by reference herein in its entirety.

### BACKGROUND

Tumor necrosis factor (TNF) is a cell signaling protein related to regulation of immune cells and apoptosis and is implicated in a variety of immune and autoimmune-mediated disorders. In particular, TNF is known to promote inflammatory response, which causes many problems associated with autoimmune disorders, such as rheumatoid arthritis, psoriatic arthritis, ankylosing spondylitis, Crohn's disease, ulcerative colitis, inflammatory bowel disease, chronic psoriasis, hidradenitis suppurativa, asthma, juvenile idiopathic arthritis, vitiligo, Graves' ophthalmopathy (also known as thyroid eye disease, or Graves' orbitopathy), and multiple sclerosis.

TNF-mediated disorders are currently treated by inhibition of TNF and, in particular, by administration of an anti-TNF agent (e.g., by anti-TNF therapy). Examples of anti-TNF agents approved in the United States include monoclonal antibodies that target TNF, such as adalimumab (Humira®), certolizumab pegol (Cimzia®), golimumab (Simponi® and Simponi Aria®), and infliximab (Remicade®), decoy circulating receptor fusion proteins such as etanercept (Enbrel®), and biosimilars, such as adalimumab ABP 501 (Amgevita™), or etanercept biosimilars GP2015 (Erelzi®).

### SUMMARY

A significant known problem with anti-TNF therapies is that response rates can be inconsistent. Indeed, recent international conferences designed to bring together leading scientists and clinicians in the fields of immunology and rheumatology to identify unmet needs in these fields almost universally identify uncertainty in response rates as an ongoing challenge. For example, the 19^{th} annual International Targeted Therapies meeting, which held break-out sessions relating to challenges in treatment of a variety of diseases, including rheumatoid arthritis, psoriatic arthritis, axial spondyloarthritis, systemic lupus erythematous, and connective tissue diseases (e.g., Sjogren's syndrome, Systemic sclerosis, vasculitis including Bechet's and IgG4 related disease), identified certain issues common to all of these diseases, specifically, “the need for better understanding the heterogeneity within each disease . . . so that predictive tools for therapeutic responses can be developed. See Winthrop, etal. “The unmet need in rheumatology: Reports from the targeted therapies meeting 2017,” Clin. Immunol. pii: S1521-6616 (17) 30543-0, Aug. 12, 2017, which is incorporated herein by reference for all purposes. Similarly, extensive literature relating to treatment of Crohn's Disease with anti-TNF therapy consistently bemoanserratic response rates and inability to predict which patients will benefit. See, e.g., M. T. Abreu, “Anti-TNF Failures in Crohn's Disease,” Gastroenterol Hepatol (N.Y.), 7 (1):37-39 (January 2011); see also Ding et al. “Systematic review: predicting and optimising response to anti-TNF therapy in Crohn's disease-algorithm for practical management,” Aliment Pharmacol. Ther., 43 (1):30-51 (January 2016) (reporting that “[p]rimary nonresponse to anti-TNF treatment affects 13-40% of patients.”), which are incorporated herein by reference for all purposes.

Thus, a significant number of patients to whom anti-TNF therapy is currently being administered do not benefit from the treatment and may even be harmed. A risk of serious infection and malignancy associated with anti-TNF therapy are so significant that product approvals may require so-called “black box warnings” be included on the label. Other potential side effects of such therapy include, for example, congestive heart failure, demyelinating disease, and other systemic side effects. Furthermore, given that several weeks to months of treatment are required before a patient is identified as not responding to anti-TNF therapy (e.g., is a non-responder to anti-TNF therapy), proper treatment of such patients can be significantly delayed as a result of the current inability to identify responder vs. non-responder subjects. See, e.g., Roda et al. “Loss of Response to Anti-TNFs: Definition, Epidemiology, and Management,” Clin. Tranl. Gastroenterol., 7 (1): e135 (January 2016) (citing Hanauer et al. “ACCENT I Study group. Maintenance Infliximab for Crohn's disease: the ACCENT I randomized trial,” Lancet 59:1541-1549 (2002); Sands et al. “Infliximab maintenance therapy for fistulizing Crohn's disease,” N. Engl. J. Med. 350:876-885 (2004)), which is incorporated herein by reference for all purposes.

Taken together, particularly given that these anti-TNF therapies can be quite expensive (typically costing upwards of $40,000-$60,000 per patient per year), these challenges make clear that technologies capable of defining, identifying, or characterizing responder vs. non-responder patient populations can represent a significant technological advance and can provide significant value to patients and to the healthcare industry more broadly. For example, the present disclosure may benefit patients, doctors, regulatory agencies, and drug developers. The present disclosure provides such technologies.

In some aspects, the methods and compositions described herein permit care providers to distinguish subjects likely to benefit from anti-TNF therapy from those who are not, reduce risks to patients, increase timing and quality of care for non-responder patient populations, increase efficiency of drug development, and avoid costs associated with administering ineffective therapy to non-responder patients or with treating side effects such patients experience upon receiving anti-TNF therapy.

In some aspects, the methods and compositions described herein embody or arise from, among other things, certain insights that include, for example, identification of the source of a problem with other methods to defining responder vs. non-responder populations or that represent particularly useful strategies for defining classifiers that distinguish between such populations. For example, as described herein, the present disclosure identifies that one source of a problem with many other methods for defining responder vs. non-responder populations through consideration of gene expression differences in the populations is that they may prioritize or otherwise focus on highest fold changes; the present disclosure teaches that such an approach misses subtle but meaningful differences relevant to disease biology. Moreover, the present disclosure offers an insight that mapping of genes with altered expression levels onto a human interactome map (in particular onto a human interactome map that represents experimentally supported physical interactions between cellular components which, in some embodiments, explicitly excludes any theoretical, calculated, or other interaction that has been proposed but not experimentally validated), can provide a useful and effective classifier for defining responders vs. non-responders to anti-TNF therapy. In some embodiments, genes included in such a classifier represent a connected module on the human interactome.

In some embodiments, the present disclosure provides a method of treating subjects suffering from a disease, disorder, or condition (e.g., inflammatory bowel disease, ulcerative colitis or Crohn's disease) with an anti-TNF therapy, the method comprising: administering the anti-TNF therapy to subjects who have been determined to display a gene expression response signature established to distinguish between responsive and non-responsive prior subjects who have received the anti-TNF therapy (e.g., where “prior subjects” refer to subjects who have previously received the anti-TNF therapy, and have been classified as responsive or non-responsive to said anti-TNF therapy).

In some embodiments, the present disclosure provides a method of treating a subject suffering from a disease, disorder, or condition (e.g., inflammatory bowel disease, ulcerative colitis or Crohn's disease) with an anti-TNF therapy, the method comprising: administering the anti-TNF therapy to subjects who have been determined not to display a gene expression response signature established to distinguish between responsive and non-responsive prior subjects who have received the anti-TNF therapy.

In some embodiments, the present disclosure provides a method of treating a subject suffering from a disease, disorder, or condition (e.g., inflammatory bowel disease, ulcerative colitis or Crohn's disease) with an anti-TNF therapy, the method comprising: administering the anti-TNF therapy to subjects who have been determined to be responsive via a classifier determined to distinguish between responsive and non-responsive subjects who have received the anti-TNF therapy (“prior subjects”), wherein the classifier distinguishes between responsive and non-responsive subjects on the basis of a set of variables, the set of variables comprising expression of one or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPM1A, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPL1, TMEM97, TLR2, FPR2, IL1B, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGR1CP, FCGR1B, ABL2, GALNT15, FFAR2, ATG4B, FST, NFIL3, RPIA, SMC2, IFIT2, NINJ1, DYRKIA, IRAK2, NUP88, or CHST10.

In some embodiments, the present disclosure provides a method of validating response to an anti-TNF therapy in a subject, the method comprising: receiving, by a processor of a computing device, a gene expression response signature determined to distinguish between responsive and non-responsive subjects to the anti-TNF therapy; analyzing, by the processor, gene expression levels of the subject relative to the gene expression response signature to determine whether the subject expresses the gene expression response signature.

In some embodiments, the present disclosure provides a method of validating response to an anti-TNF therapy in a subject, the method comprising: receiving, by a processor of a computing device, a gene expression response signature determined to distinguish between responsive and non-responsive subjects to the anti-TNF therapy; analyzing, by the processor, gene expression levels of the subject relative to the gene expression response signature to determine whether the subject expresses the gene expression response signature, wherein the gene expression response signature comprises one or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPMIA, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPLI, TMEM97, TLR2, FPR2, IL1B, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGRICP, FCGRIB, ABL2, GALNT15, FFAR2, ATG4B, FST, NFIL3, RPIA, SMC2, IFIT2, NINJ1, DYRKIA, IRAK2, NUP88, or CHST10.

In some embodiments, the present disclosure provides a system for determining or validating responsiveness to anti-TNF therapy for a subject suffering from a disease, the system comprising a processor of a computing device; and a memory having instructions stored thereon, wherein the instructions, when executed by the processor cause the processor to perform: receiving, by the processor, a gene expression response signature determined to distinguish between responsive and non-responsive subjects to the anti-TNF therapy; analyzing, by the processor, gene expression levels of the subject relative to the gene expression response signature to determine whether the subject expresses the gene expression response signature.

In some embodiments, the present disclosure provides a system for determining or validating responsiveness to anti-TNF therapy for a subject suffering from a disease, the system comprising a processor of a computing device; and a memory having instructions stored thereon, wherein the instructions, when executed by the processor cause the processor to perform: receiving, by the processor, a gene expression response signature determined to distinguish between responsive and non-responsive subjects to the anti-TNF therapy; analyzing, by the processor, gene expression levels of the subject relative to the gene expression response signature to determine whether the subject expresses the gene expression response signature, wherein the gene expression response signature comprises one or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPMIA, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPLI, TMEM97, TLR2, FPR2, ILIB, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGRICP, FCGRIB, ABL2, GALNT15, FFAR2, ATG4B, FST, NFIL3, RPIA, SMC2, IFIT2, NINJ1, DYRKIA, IRAK2, NUP88, or CHST10.

In some embodiments, the present disclosure provides a method of treating subjects suffering from a disease, disorder, or condition with an alternative to anti-TNF therapy, the method comprising: administering the alternative to anti-TNF therapy to the subject who have been determined to be non-responsive via a classifier determined to distinguish between responsive and non-responsive subjects who have received the anti-TNF therapy (“prior subjects”), and the classifier distinguishes between responsive and non-responsive subjects on the basis of a set of variables.

In some embodiments, the present disclosure provides a method of treating subjects suffering from a disease, disorder, or condition with an alternative to anti-TNF therapy, the method comprising: administering the alternative to anti-TNF therapy to the subject who have been determined to be non-responsive via a classifier determined to distinguish between responsive and non-responsive subjects who have received the anti-TNF therapy (“prior subjects”), and the classifier distinguishes between responsive and non-responsive subjects on the basis of a set of variables, the set of variables comprising expression of one or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPMIA, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPLI, TMEM97, TLR2, FPR2, ILIB, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGR1CP, FCGR1B, ABL2, GALNT15, FFAR2, ATG4B, FST, NFIL3, RPIA, SMC2, IFIT2, NINJ1, DYRKIA, IRAK2, NUP88, or CHST10.

In some embodiments, the present disclosure provides a kit for evaluating a likelihood that a subject suffering from an autoimmune disorder will not respond to an anti-TNF therapy, the kit comprising a set of reagents for detecting an expression level of one or more genes.

In some embodiments, the present disclosure provides a kit for evaluating a likelihood that a subject suffering from an autoimmune disorder will not respond to an anti-TNF therapy, the kit comprising a set of reagents for detecting an expression level of one or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPMIA, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPLI, TMEM97, TLR2, FPR2, ILIB, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGR1CP, FCGR1B, ABL2, GALNT15, FFAR2, ATG4B, FST, NFIL3, RPIA, SMC2, IFIT2, NINJ1, DYRK1A, IRAK2, NUP88, or CHST10.

Additional aspects and advantages of the present disclosure will become readily apparent from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-1D depicts identification of response discriminatory genes. (A) depicts Pearson correlation distribution of gene expression values with response outcomes in observed versus randomized gene expression data. The signal-to-noise ratio of actual and randomized Pearson correlations were derived by dividing the randomized valued by the observed value. (B) depicts genes associated to top 123 probes with highest signal-to-noise ratio were mapped on the network resulting in observation of multiple connected components. Larger nodes correspond to genes with higher signal-to-noise ratio ranks, and node colors indicate expression change in responders with respect to inadequate responders. (C) depicts comparison of the observed average shortest path between the genes associated to top 123 probes with the expected average shortest path generated by 100,000 randomizations. (D) depicts a heatmap representing the baseline gene expression values of LCC genes (CXCL1 (204470_at), CXCL2 (209774_x_at), MAP3K20 (225662_at), MEIS1 (1559477_s_at), CEBPB (212501_at), CXCL6 (206336_at), MS4A7 (223344_s_at), DRAM1 (218627_at), NR3C1 (201865_x_at), IGFBP5 (203424_s_at), (211959_at), AMIGO2 (222108_at), MMP12 (204580_at)) used for classifier training across patients. Red corresponds to higher relative expression values and green corresponds to lower relative expression values;

FIG. 2A-2C depicts a cross-cohort performance of the response prediction classifier. (A) depicts a receiver operating characteristic (ROC) curve. (B) depicts classifier predicted scores among validation set patients who are true clinical responders (R) or inadequate responders (IR) to infliximab. (C) depicts accuracy in predicting inadequate responders to infliximab in the validation cohort. The classifier is able to detect 50% of the non-responders with 100% accuracy;

FIG. 3A-3C depicts differential gene expression of response discriminatory genes. (A) depicts a Volcano plot indicating the differential expression for all genes between responders and inadequate responders to infliximab. The 12 genes (corresponding to 13 probes) in the classifier LCC are highlighted in orange. (B) depicts a comparison of the expression of the 12 largest connected component (LCC) classifier genes (CXCL1 (204470_at), CXCL2 (209774_x_at), MAP3K20 (225662_at), MEIS1 (1559477_s_at), CEBPB (212501_at), CXCL6 (206336_at), MS4A7 (223344_s_at), DRAM1 (218627_at), NR3C1 (201865_x_at), IGFBP5 (203424_s_at), (211959_at), AMIGO2 (222108_at), MMP12 (204580_at)) of responders or inadequate responders vs. healthy controls. (C) depicts Unsupervised hierarchical cluster analysis of 12 classifier largest connected component (LCC) genes illustrating the relative RNA expression between healthy controls, responders and inadequate responders;

FIG. 4A-4B depicts apparently distinct gene lists mapped onto the same network region of the Human Interactome indicated a common underlying biology of response. (A) depicts a response module: largest connected component formed by the proteins encoded by the response signature genes from the training and validation cohorts. Proteins encoded by training cohort genes are in blue and those encoded by validation cohort genes are in orange. (B) depicts a distribution of largest connected component (LCC) size from random expectation;

FIG. 5 depicts selection of the number of probes to be considered when calculating largest connected component (LCC) size. This plot summarizes the LCC size with respect to the number of top differentially expressed probes to be considered. The LCC reached a plateau between 123 and 199 probes with an LCC size of 12;

FIG. 6 depicts an example network environment and computing devices for use in some embodiments;

FIG. 7 depicts an example workflow for developing a classifier in some embodiments;

FIG. 8A-8D depict plots illustrating in-cohort rheumatoid arthritis (RA) classifier validation using leave-one-out cross validation when training on Feature Set 1 (FIGS. 4A and 4B) and top nine signature genes (FIGS. 4C and 4D). FIG. 4B depicts Negative Predictive Value (NPV) vs. specificity. FIG. 4D depicts Negative Predictive Value (NPV) vs. specificity; and

FIG. 9A-9B depict ROC curves of cross cohort classifier test results (in FIG. 5A) and negative predictive performance (NPV) (in FIG. 5B) for the RA classifier.

FIG. 10 depicts relationships between positive predictive value (PPV), negative predictive value (NPV), sensitivity, and specificity.

### DETAILED DESCRIPTION

While various embodiments of the present disclosure have been shown and described herein, such embodiments are provided by way of example only. Numerous variations, changes, and substitutions may occur without departing from the present disclosure. It should be understood that various alternatives to the embodiments of the disclosure described herein may be employed.

As noted, the response rate for patients undergoing anti-TNF therapy can be inconsistent. Technologies that reliably identify responsive or non-responsive subjects may be beneficial, as they may avoid wasteful and even potentially damaging administration of therapy to subjects who will not respond, and furthermore may allow timely determination of more appropriate treatment for such subjects. The present disclosure provides such technologies, addressing needs of patients, their families, drug developers, and medical professionals each of whom may suffer under the current system.

While significant effort has been invested in efforts to develop technologies that reliably predict responsiveness (e.g., by identifying responsive vs. non-responsive populations) or development of resistance for certain therapeutic agents, regimens, or modalities, success has been elusive and almost exclusively limited to the oncology sector. Complex disorders, such as autoimmune or cardiovascular diseases, can be particularly challenging.

Cancer is generally associated with particular strong driver genes, which dramatically simplifies the analysis required to identify responder vs non-responder patient populations and significantly improves success rates. By contrast, diseases associated with more complex genetic (or epigenetic) contributions have thus far presented an insurmountable challenge for available technologies.

Indeed, a large number of published reports that describe efforts to develop technologies for predicting responsiveness to anti-TNF therapy in inflammatory conditions (e.g., rheumatoid arthritis) most commonly rely on blood-based gene expression classifiers. See, e.g., Nakamura et al. “Identification of baseline gene expression signatures predicting therapeutic responses to three biologic agents in rheumatoid arthritis: a retrospective observational study” Arthritis Research & Therapy (2016) 18:159 DOI 10.1186/s13075-016-1052-8, which is incorporated herein by reference for all purposes. However, a clinically utilizable classifier has not yet been identified. Notably, Toonen et al. performed an independent study that tested eight different gene expression signatures predicting response to anti-TNF, and reported that most signatures failed to demonstrate sufficient predictive value to be of utility. See M. Toonen et al. “Validation Study of Existing Gene Expression Signatures for Anti-TNF Treatment in Patients with Rheumatoid Arthritis,” PLOS ONE 7 (3): e33199, which is incorporated herein by reference for all purposes. Thomson et al. attempted to describe a blood-based classifier to identify non-responders to one anti-TNF therapy, infliximab, in rheumatoid arthritis. Thomson et al. “Blood-based identification of non-responders to anti-TNF therapy in rheumatoid arthritis,” BMC Med Genomics, 8:26, *1-12 (2015), which is incorporated herein by reference for all purposes. Their proposed classifier comprised 18 signaling mechanisms indicative of higher TNF-mediated inflammatory signaling in responders at baseline, versus higher levels of specific metabolic activities in non-responders at baseline. The test, however, did not reach the level of predictive accuracy required for commercialization and so development was stopped.

Generally, methods for defining responder vs. non-responder classifiers for anti-TNF therapy rely on machine-learning approaches, using mean values across classes of response, and focus on genes with the highest fold changes, often in a pathway-based context. The present disclosure identifies various sources of problems with these methods, and, moreover, provides technologies that solve or avoid the problems, thereby satisfying the long felt need within the community for accurate or useful predictive classifiers.

Among other things, the present disclosure appreciates that machine learning may be useful for finding correlations between datasets of patients but fails to achieve sufficient predictive accuracy across cohorts. Furthermore, the present disclosure identifies that prioritizing or otherwise focusing on highest fold changes can miss subtle but meaningful differences relevant to disease biology. Still further, the present disclosure offers an insight that mapping of genes with altered expression levels onto a human interactome (e.g., that represents experimentally supported physical interactions between cellular components and, in some embodiments, explicitly excludes any theoretical, calculated, or other interaction that has been proposed but not experimentally validated) can provide a useful and effective classifier for defining responders vs. non-responders to anti-TNF therapy. In some embodiments, genes included in such a classifier represent a connected module in the human interactome. Examples of methods of treatment and classifier development related to the present disclosure is found in WO 2019/178546 A1, which is incorporated herein by reference for all purposes.

As used herein, the term “administration” generally refers to the administration of a composition to a subject or system, for example to achieve delivery of an agent that is, or is included in or otherwise delivered by, the composition.

As used herein, the term “agent” generally refers to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.).

As used herein, the term “amino acid” generally refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. Asused herein, the term “standard amino acid” refers to any of the twenty L-amino acids commonly found in naturally occurring peptides. “Nonstandard amino acid” refers to any amino acid, other than the standard amino acids, regardless of whether it is or can be found in a natural source. In some embodiments, an amino acid, including a carboxy- or amino-terminal amino acid in a polypeptide, can contain a structural modification as compared to the general structure above. For example, in some embodiments, an amino acid may be modified by methylation, amidation, acetylation, pegylation, glycosylation, phosphorylation, or substitution (e.g., of the amino group, the carboxylic acid group, one or more protons, or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. As will be clear from context, in some embodiments, the term “amino acid” may be used to refer to a free amino acid; in some embodiments it may be used to refer to an amino acid residue of a polypeptide, e.g., an amino acid residue within a polypeptide.

As used herein, the term “analog” generally refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g, by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

As used herein, the term “antagonist” generally refers to an agent, or condition whose presence, level, degree, type, or form is associated with a decreased level or activity of a target. An antagonist may include an agent of any chemical class including, for example, small molecules, polypeptides, nucleic acids, carbohydrates, lipids, metals, or any other entity that shows the relevant inhibitory activity. In some embodiments, an antagonist may be a “direct antagonist” in that it binds directly to its target; in some embodiments, an antagonist may be an “indirect antagonist” in that it exerts its influence by mechanisms other than binding directly to its target; e.g., by interacting with a regulator of the target, so that the level or activity of the target is altered). In some embodiments, an “antagonist” may be referred to as an “inhibitor”.

As used herein, the term “antibody” generally refers to a polypeptide that includes canonical immunoglobulin sequence elements sufficient to confer specific binding to a particular target antigen. Intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, or “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops called “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. Affinity or other binding attributes of Fc regions for Fc receptors can be modulated through glycosylation or other modification. In some embodiments, antibodies produced or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region-sequences that are characteristic of mouse, rabbit, primate, or human antibodies. In some embodiments, antibody sequence elements are humanized, primatized, chimeric, etc. Moreover, the term “antibody” as used herein, can refer in appropriate embodiments (unless otherwise stated or clear from context) to any of the investigated or developed constructs or formats for utilizing antibody structural and functional features in alternative presentation. For example, an antibody utilized in accordance with the present disclosure is in a comprising intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies minibodies; BiTE®s; ankyrin repeat proteins or DARPINs®; Avimers®; DARTs; TCR-like antibodies;, Adnectins®; Affilins®; Trans-bodies®; Affibodies®; TrimerX®; MicroProteins; Fynomers®, Centyrins®; or KALBITOR®s. In some embodiments, an antibody may lack a covalent modification (e.g., attachment of a glycan) that it may have if produced naturally. In some embodiments, an antibody may contain a covalent modification (e.g, attachment of a glycan, a payload [e.g., a detectable moiety, a therapeutic moiety, a catalytic moiety, etc], or other pendant group [e.g., poly-ethylene glycol, etc.]).

As used herein, two events or entities are “associated” with one another if the presence, level, degree, type or form of one is generally correlated with that of the other. For example, a particular entity (e.g., polypeptide, genetic signature, metabolite, microbe, etc) is considered to be associated with a particular disease, disorder, or condition, if its presence, level or form correlates with incidence of or susceptibility to the disease, disorder, or condition (e.g., across a relevant population). In some embodiments, two or more entities are physically “associated” with one another if they interact, directly or indirectly, so that they are or remain in physical proximity with one another. In some embodiments, two or more entities that are physically associated with one another are covalently linked to one another; in some embodiments, two or more entities that are physically associated with one another are not covalently linked to one another but are non-covalently associated, for example by mechanism of hydrogen bonds, van der Waals interaction, hydrophobic interactions, magnetism, and combinations thereof.

As used herein, the term “biological sample” generally refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or bronchoalveolar lavages; aspirates; scrapings; bonemarrow specimens; tissue biopsy specimens; surgical specimens; feces, other body fluids, secretions, or excretions; or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate method. For example, in some embodiments, a primary biological sample is obtained by methods comprising biopsy (e.g., fine needle aspiration or tissue biopsy), surgery, or collection of body fluid (e.g., blood, lymph, feces etc.), or a combination thereof. In some embodiments, as will be clear from context, the term “sample” refers to a preparation that is obtained by processing (e.g., by removing one or more components of or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation or purification of certain components, etc.

As used herein, the term “biological network” generally refers to any network that applies to biological systems, having sub-units (e.g., “nodes”) that are linked into a whole, such as species units linked into a whole web. In some embodiments, a biological network is a protein-protein interaction network (PPI), representing interactions among proteins present in a cell, where proteins are nodes and their interactions are edges. In some embodiments, connections between nodes in a PPI are experimentally verified. In some embodiments, connections between nodes are a combination of experimentally verified a mathematically calculated. In some embodiments, a biological network is a human interactome (a network of experimentally derived interactions that occur in human cells, which includes protein-protein interaction information as well as gene expression and co-expression, cellular co-localization of proteins, genetic information, metabolic and signaling pathways, etc.). In some embodiments, a biological network is a gene regulatory network, a gene co-expression network, a metabolic network, or a signaling network.

As used herein, the term “combination therapy” generally refers to a clinical intervention in which a subject is simultaneously exposed to two or more therapeutic regimens (e.g., two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), or at different time points. In some embodiments, two or more therapeutic agents may be administered together in a combination composition, or even in a combination compound (e.g., as part of a single chemical complex or covalent entity), via the same administration route, or at the same time.

As used herein, the term “comparable” generally refers to two or more agents, entities, situations, sets of conditions, etc., that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those practicing the present disclosure will understand, in context, what degree of identity is required in any given circumstance for two or more such agents, entities, situations, sets of conditions, etc. to be considered comparable. For example, sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, the phrase “corresponding to” generally refers to a relationship between two entities, events, or phenomena that share sufficient features to be reasonably comparable such that “corresponding” attributes are apparent. For example, in some embodiments, the term may be used in reference to a compound or composition, to designate the position or identity of a structural element in the compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, for purposes of simplicity, residues in a polypeptide are often designated using a canonical numbering system based on a reference related polypeptide, so that an amino acid “corresponding to” a residue at position 190, for example, may not actually be the 190th amino acid in a particular amino acid chain but rather corresponds to the residue found at 190 in the reference polypeptide; those practicing the present disclosure will readily appreciate how to identify “corresponding” amino acids. For example, those practicing the present disclosure will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides or nucleic acids in accordance with the present disclosure.

As used herein, the term “dosing regimen” generally refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (e.g., is a therapeutic dosing regimen).

As used herein, the terms “improved,” “increased,” or “reduced,”, or grammatically comparable comparative terms thereof, generally indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.).

As used herein, the term “patient” or “subject” generally refers to any organism to which a provided composition is or may be administered, e.g., for experimental, diagnostic, prophylactic, cosmetic, or therapeutic purposes. Typical patients or subjects include animals (e.g., mammals such as mice, rats, rabbits, non-human primates, or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose or to treat a disease, disorder, or condition.

As used herein, the term “pharmaceutical composition” generally refers to an active agent, formulated together with one or more pharmaceutically acceptable carriers. In some embodiments, the active agent is present in unit dose amounts appropriate for administration in a therapeutic regimen to a relevant subject (e.g., in amounts that have been demonstrated to show a statistically significant probability of achieving a predetermined therapeutic effect when administered), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence or magnitude sufficient to achieve statistical relevance). Those practicing the present disclosure will be aware, or will readily be able to determine, in a given context, a degree or prevalence of difference that is required or sufficient to achieve such statistical significance.

As used herein, the phrase “pharmaceutically acceptable” generally refers to those compounds, materials, compositions, or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “responder” generally refers to a subject that displays an improvement in clinical signs and symptoms after receiving anti-TNF therapy for a period of time. Those practicing the present disclosure will understand that the medical community may establish an appropriate period of time for any particular disease or condition, or for any particular patient or patient type. To give but a few examples, in some embodiments, the period of time may be at least 8 weeks. In some embodiments, the period of time may be at least 12 weeks. In some embodiments, the period of time may be 14 weeks.

As used herein, the term “non-responder” generally refers to a subject that displays a insufficient improvement in clinical signs and symptoms after receiving anti-TNF therapy for a period of time. Those practicing the present disclosure will understand that the medical community may establish an appropriate period of time for any particular disease or condition, or for any particular patient or patient type. To give but a few examples, in some embodiments, the period of time may be at least 8 weeks. In some embodiments, the period of time may be at least 12 weeks. In some embodiments, the period of time may be 14 weeks.

As used herein, the term “reference” generally describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested or determined substantially simultaneously with the testing or determination of interest. In some embodiments, a reference or control is a historical reference or control, optionally embodied in a tangible medium. Typically, a reference or control is determined or characterized under comparable conditions or circumstances to those under assessment. Those practicing the present disclosure will appreciate when sufficient similarities are present to justify reliance on or comparison to a particular possible reference or control.

As used herein, the phrase “therapeutic agent” generally refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, or reduce incidence of one or more symptoms or features of a disease, disorder, or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

As used herein, the term “therapeutically effective amount” generally refers to an amount of a substance (e.g., a therapeutic agent, composition, or formulation) that elicits a desired biological response when administered as part of a therapeutic regimen. In some embodiments, a therapeutically effective amount of a substance is an amount that is sufficient, when administered to a subject suffering from or susceptible to a disease, disorder, or condition, to treat, diagnose, prevent, or delay the onset of the disease, disorder, or condition. As will be appreciated by those practicing the present disclosure, the effective amount of a substance may vary depending on such factors as the desired biological endpoint, the substance to be delivered, the target cell or tissue, etc. For example, the effective amount of compound in a formulation to treat a disease, disorder, or condition is the amount that alleviates, ameliorates, relieves, inhibits, prevents, delays onset of, reduces severity of or reduces incidence of one or more symptoms or features of the disease, disorder or condition. In some embodiments, a therapeutically effective amount is administered in a single dose; in some embodiments, multiple unit doses are required to deliver a therapeutically effective amount.

As used herein, the terms “treat,” “treatment,” or “treating” generally refer to any method used to partially or completely alleviate, ameliorate, relieve, inhibit, prevent, delay onset of, reduce severity of, or reduce incidence of one or more symptoms or features of a disease, disorder, or condition. Treatment may be administered to a subject who does not exhibit signs of a disease, disorder, or condition. In some embodiments, treatment may be administered to a subject who exhibits only early signs of the disease, disorder, or condition, for example, for the purpose of decreasing the risk of developing pathology associated with the disease, disorder, or condition.

As used herein, the “term positive predictive value (PPV)” generally refers to a probability that a person with a positive test actually has a disease, disorder, or condition. In some cases, PPV is associated with a non-responder when the disease, disorder, or condition is ulcerative colitis. In some cases, PPV is associated with a responder when the disease, disorder, or condition is rheumatoid arthritis. As used herein, the term “negative predictive value (NPV)” generally refers to a probability that a person with a negative test actually does not have a disease, disorder, or condition. In some cases, NPV is associated with a responder when the disease, disorder, or condition is ulcerative colitis. In some cases, NPV is associated with a non-responder when the disease, disorder, or condition is rheumatoid arthritis. As used herein, the term “true positive rate (TPR)” or sensitivity generally refers to a test's ability to correctly identify all people who have a disease, disorder, or condition. In some cases, TPR is associated with a non-responder when the disease, disorder, or condition is ulcerative colitis. In some cases, TPR is associated with a responder when the disease, disorder, or condition is rheumatoid arthritis. As used herein, the term “true negative rate (TNR or NPR)” or specificity generally refers to a test's ability to correctly identify all people who do not have a disease, disorder, or condition. In some cases, TNR is associated with a responder when the disease, disorder, or condition is ulcerative colitis. In some cases, TNR is associated with a non-responder when the disease, disorder, or condition is rheumatoid arthritis.

### Anti-TNF Therapy

TNF-mediated disorders are currently treated by inhibition of TNF and, in particular, by administration of an anti-TNF agent (e.g., by anti-TNF therapy). Examples of anti-TNF agents approved for use in the United States include monoclonal antibodies such as adalimumab (Humira®), certolizumab pegol (Cimzia®), infliximab (Remicade), and decoy circulating receptor fusion proteins such as etanercept (Enbrel®). These agents are currently approved for use in treatment of indications, according to dosing regimens, as set forth below in Table 1.

TABLE 1

| | | Certolizumab | | | | |
|---|---|---|---|---|---|---|
| Indication | Adalimumab¹ | Pegol¹ | Infliximab² | Etanercept¹ | Golimumab¹ | Golimumab² |
| Juvenile | 10 kg (22 lbs) | N/A | N/A | 0.8 mg/kg | N/A | N/A |
| Idiopathic | to <15 kg (33 | | | weekly, with a | | |
| Arthritis | lbs): 10 mg | | | maximum of 50 | | |
| | every other | | | mg per week | | |
| | week | | | | | |
| | 15 kg (33 lbs) | | | | | |
| | to <30 kg (66 | | | | | |
| | lbs): 20 mg | | | | | |
| | every other | | | | | |
| | week | | | | | |
| | ≥30 kg (66 | | | | | |
| | lbs): 40 mg | | | | | |
| | every other | | | | | |
| | week | | | | | |
| Psoriatic | 40 mg every other | 400 mg initially | 5 mg/kg at 0, 2 | 50 mg once | 50 mg | N/A |
| Arthritis | week | and at week 2 and | and 6 weeks, then | weekly with or | administered by | |
| | | 4, followed by | every 8 weeks | without | subcutaneous | |
| | | 200 mg every | | methotrexate | injection once a | |
| | | other week; for | | | month | |
| | | maintenance | | | | |
| | | dosing, 400 mg | | | | |
| | | every 4 weeks | | | | |
| Rheumatoid | 40 mg every other | 400 mg initially | In conjunction | 50 mg once | 50 mg once a | 2 mg/kg |
| Arthritis | week | and at Weeks 2 | with | weekly with or | month | intravenous |
| | | and 4, followed | methotrexate, 3 | without | | infusion over 30 |
| | | by 200 mg every | mg/kg at 0, 2 and | methotrexate | | minutes at |
| | | other week; for | 6 weeks, then | | | weeks 0 and 4, |
| | | maintenance | every 8 weeks | | | then every 8 |
| | | dosing, 400 mg | | | | weeks |
| | | every 4 weeks | | | | |
| Ankylosing | 40 mg every other | 400 mg (given as | 5 mg/kg at 0, 2 | 50 mg once | 50 mg | N/A |
| Spondylitis | week | 2 subcutaneous | and 6 weeks, then | weekly | administered by | |
| | | injections of 200 | every 6 weeks | | subcutaneous | |
| | | mg each) initially | | | injection once a | |
| | | and at weeks 2 | | | month | |
| | | and 4, followed | | | | |
| | | by 200 mg every | | | | |
| | | other week or 400 | | | | |
| | | mg every 4 weeks | | | | |
| Adult | Initial dose | 400 mg initially | 5 mg/kg at 0, 2 | N/A | N/A | N/A |
| Crohn's | (Day 1): 160 | and at Weeks 2 | and 6 weeks, then | | | |
| Disease | mg | and 4 | every 8 weeks. | | | |
| | Second dose | Continue with | | | | |
| | two weeks | 400 mg every | | | | |
| | later (Day 15): | four weeks | | | | |
| | 80 mg | | | | | |
| | Two weeks | | | | | |
| | later (Day 29): | | | | | |
| | Begin a | | | | | |
| | maintenance | | | | | |
| | dose of 40 mg | | | | | |
| | every other | | | | | |
| | week | | | | | |
| Pediatric | 17 kg (37 lbs) | N/A | 5 mg/kg at 0, 2 | N/A | N/A | N/A |
| Crohn's | to <40 kg (88 lbs): | | and 6 weeks, then | | | |
| Disease | Initial dose | | every 8 weeks. | | | |
| | (Day 1): 80 mg | | | | | |
| | Second dose | | | | | |
| | two weeks | | | | | |
| | later (Day 15): | | | | | |
| | 40 mg | | | | | |
| | Two weeks | | | | | |
| | later (Day 29): | | | | | |
| | Begin a | | | | | |
| | maintenance | | | | | |
| | dose of 20 mg | | | | | |
| | every other | | | | | |
| | week ≥40 kg | | | | | |
| | (88 lbs): | | | | | |
| | Initial dose | | | | | |
| | (Day 1): 160 | | | | | |
| | mg | | | | | |
| | Second dose | | | | | |
| | two weeks | | | | | |
| | later (Day 15): | | | | | |
| | 80 mg | | | | | |
| | Two weeks | | | | | |
| | later (Day 29): | | | | | |
| | Begin a | | | | | |
| | maintenance | | | | | |
| | dose of 40 mg | | | | | |
| | every other | | | | | |
| | week | | | | | |
| Ulcerative | Initial dose | N/A | 5 mg/kg at 0, 2 | N/A | N/A | N/A |
| Colitis | (Day 1): 160 | | and 6 weeks, then | | | |
| | mg | | every 8 weeks. | | | |
| | Second dose | | | | | |
| | two weeks | | | | | |
| | later (Day 15): | | | | | |
| | 80 mg | | | | | |
| | Two weeks | | | | | |
| | later (Day 29): | | | | | |
| | Begin a | | | | | |
| | maintenance | | | | | |
| | dose of 40 mg | | | | | |
| | every other | | | | | |
| | week | | | | | |
| Plaque | 80 mg initial | N/A | N/A | 50 mg twice | N/A | N/A |
| Psoriasis | dose; 40 mg | | | weekly for 3 | | |
| | every other week | | | months, followed | | |
| | beginning one | | | by 50 mg once | | |
| | week after initial | | | weekly | | |
| | dose | | | | | |
| Hidradenitis | Initial dose (Day | N/A | N/A | N/A | N/A | N/A |
| Suppurativa | 1): 160 mg | | | | | |
| | Second dose | | | | | |
| | two weeks later | | | | | |
| | (Day 15): 80 mg | | | | | |
| | Third dose (Day | | | | | |
| | 29) and | | | | | |
| | subsequent doses: | | | | | |
| | 40 mg every | | | | | |
| | week | | | | | |
| Uveitis | 80 mg initial | N/A | N/A | N/A | N/A | N/A |
| | dose; | | | | | |
| | 40 mg every other | | | | | |
| | week beginning | | | | | |
| | one week after | | | | | |
| | initial dose | | | | | |

¹Administered by subcutaneous injection.
²Administered by intravenous infusion.

The present disclosure provides technologies relevant to anti-TNF therapy, including those therapeutic regimens as set forth in Table 1. In some embodiments, the anti-TNF therapy is or comprises administration of infliximab (Remicade®), adalimumab (Humira®), certolizumab pegol (Cimzia®), etanercept (Enbrel®), or biosimilars thereof. In some embodiments, the anti-TNF therapy is or comprises administration of infliximab (Remicade®) or adalimumab (Humira®). In some embodiments, the anti-TNF therapy is or comprises administration of infliximab (Remicade®). In some embodiments, the anti-TNF therapy is or comprises administration of adalimumab (Humira®).

In some embodiments, the anti-TNF therapy is or comprises administration of a biosimilar anti-TNF agent. In some embodiments, the anti-TNF agent comprises infliximab biosimilars such as CT-P13, BOW015, SB2, Inflectra®, Renflexis®, and Ixifi™, adalimumab biosimilars such as ABP 501 (Amgevita™), Adfrar®, and Hulio™ and etanercept biosimilars such as HD203, SB4 (Benepali®), GP2015, Erelzi®, Intacept®, or a combination thereof.

In some embodiments, the present disclosure defines patient populations to whom anti-TNF therapy can (or cannot) be administered. In some embodiments, technologies provided by the present disclosure generate information useful to doctors, pharmaceutical companies, payers, or regulatory agencies who wish to ensure that anti-TNF therapy is administered to responder populations or is not administered to non-responder populations.

### Diseases, Disorders or Conditions

In general, provided disclosures are useful in any context in which administration of anti-TNF therapy is contemplated or implemented. In some embodiments, provided technologies are useful in the diagnosis or treatment of subjects suffering from a disease, disorder, or condition associated with aberrant (e.g., elevated) TNF expression or activity. In some embodiments, provided technologies are useful in monitoring subjects who are receiving or have received anti-TNF therapy. In some embodiments, provided technologies identify whether a subject will or will not respond to a given anti-TNF therapy. In some embodiments, the provided technologies identify whether a subject will develop resistance to a given anti-TNF therapy.

Accordingly, the present disclosure provides technologies relevant to treatment of the various diseases, disorders, and conditions related to TNF, including those listed in Table 1. In some embodiments, a subject is suffering from a disease, disorder, or condition comprising rheumatoid arthritis, psoriatic arthritis, ankylosing spondylitis, Crohn's disease (adult or pediatric), ulcerative colitis, inflammatory bowel disease, chronic psoriasis, plaque psoriasis, hidradenitis suppurativa, asthma, uveitis, juvenile idiopathic arthritis, vitiligo, Graves' ophthalmopathy (see also thyroid eye disease, or Graves' orbitopathy), multiple sclerosis, or a combination thereof. In some embodiments, the disease, disorder, or condition is rheumatoid arthritis. In some embodiments, the disease, disorder, or condition is psoriatic arthritis. In some embodiments, the disease, disorder, or condition is ankylosing spondylitis. In some embodiments, the disease, disorder, or condition is Crohn's disease. In some embodiments, the disease, disorder, or condition is adult Crohn's disease. In some embodiments, the disease, disorder, or condition is pediatric Crohn's disease. In some embodiments, the disease, disorder, or condition is inflammatory bowel disease. In some embodiments, the disease, disorder, or condition is ulcerative colitis. In some embodiments, the disease, disorder, or condition is chronic psoriasis. In some embodiments, the disease, disorder, or condition is plaque psoriasis. In some embodiments, the disease, disorder, or condition is hidradenitis suppurativa. In some embodiments, the disease, disorder, or condition is asthma. In some embodiments, the disease, disorder, or condition is uveitis. In some embodiments, the disease, disorder, or condition is juvenile idiopathic arthritis. In some embodiments, the disease, disorder, or condition is vitiligo. In some embodiments, the disease, disorder, or condition is Graves' ophthalmopathy (see also thyroid eye disease, or Graves' orbitopathy). In some embodiments, the disease, disorder, or condition is multiple sclerosis.

### Provided Classifier(s)

The present disclosure provides classifiers that are or comprise gene expression response signatures that identify (e.g., predict) which patients will or will not respond to anti-TNF therapy. In some embodiments, a gene classifier comprises a gene expression response signature (e.g., a set of one or more genes) that distinguishes between responsive and non-responsive prior subjects (e.g., where “prior subjects” refers to subjects who have previously received an anti-TNF therapy, and have been classified as responders or non-responders).

As described herein, the present disclosure provides gene expression response signatures and methods for determining gene expression response signatures that are characteristic of anti-TNF responder or non-responder populations. In some embodiments, a particular gene expression response signature classifies responder or non-responder populations for a particular anti-TNF therapy (e.g., a particular anti-TNF agent or regimen). In some embodiments, a particular gene expression response signature classifies responder or non-responder populations suffering from a particular disease, disorder, or condition, for a particular anti-TNF therapy (e.g., a particular anti-TNF agent or regimen). In some embodiments, responder or non-responder populations for different anti-TNF therapies (e.g., different anti-TNF agents or regimens) may overlap or be co-extensive; in some such embodiments, the present disclosure may provide gene expression response signatures that serve as gene classifiers for responder or non-responder populations across anti-TNF therapies.

In some embodiments, as described herein, a gene expression response signature is identified by retrospective analysis of gene expression levels in biological samples from subjects who have received anti-TNF therapy (e.g., “prior subjects”) and have been determined to respond (e.g., are responders) or not to respond (e.g., are non-responders). In some embodiments, all such subjects have received the same anti-TNF therapy (optionally for the same or different periods of time); alternatively or additionally, in some embodiments, all such subjects have been diagnosed with the same disease, disorder or condition. In some embodiments, subjects whose biological samples are analyzed in the retrospective analysis had received different anti-TNF therapy (e.g., with a different anti-TNF agent or according to a different regimen); alternatively or additionally, in some embodiments, subjects whose biological samples are analyzed in the retrospective analysis have been diagnosed with different diseases, disorders, or conditions.

In some embodiments, a gene expression response signature as described herein is determined by comparison of gene expression levels in the responder vs. non-responder populations whose biological samples are analyzed in a retrospective analysis as described herein. In some embodiments, a gene expression response signature comprises genes whose individual expression levels show statistically significant differences between the responder and non-responder populations. In some embodiments, a gene expression response signature comprises genes whose linear combination of expression levels show statistically significant differences between the responder and non-responder populations. In some embodiments, a gene expression response signature comprises genes whose non-linear combination of expression levels show statistically significant differences between the responder and non-responder populations.

In some embodiments, a gene expression response signature is incorporated into a classifier for distinguishing between responder and non-responder subjects. In some embodiments, a classifier is developed by assessing each of: the one or more genes whose expression levels significantly correlate (e.g., in a linear or non-linear manner) to clinical responsiveness or non-responsiveness (e.g., a gene expression response signature); and optionally one or more of the presence of the one or more single nucleotide polymorphs (SNPs) and at least one clinical characteristic.

In some embodiments, the present disclosure embodies an insight that the source of a problem with certain efforts to identify or provide gene expression response signatures through comparison of gene expression levels in responder vs non-responder populations have emphasized or focused on (often solely on) genes that show the largest difference (e.g., greater than 2-fold change) in expression levels between the populations. The present disclosure appreciates that even genes those expression level differences are relatively small (e.g., less than 2-fold change in expression) provide useful information if the difference is significant, and are valuably included in a gene expression response signature in embodiments described herein.

Moreover, in some embodiments, the present disclosure embodies an insight that analysis of interaction patterns of genes whose expression levels show statistically significant differences (optionally including small differences) between responder and non-responder populations as described herein provides new and valuable information that materially improves the quality and predictive power of a gene expression response signature.

Further, as noted, the present disclosure provides technologies that allow practitioners to reliably and consistently predict response to anti-TNF therapy in a cohort of subjects (e.g., treatment naïve subjects, e.g., subjects who have not received anti-TNF therapy). In particular, for example, the rate of response for some anti-TNF therapies is less than 35% within a given cohort of subjects. The provided technologies allow for prediction of greater than 65% accuracy within a cohort of subjects a response rate (e.g., whether certain subjects will or will not respond to a given therapy). In some embodiments, the methods and systems described herein predict 65% or greater the subjects that are responders (e.g., will respond to anti-TNF therapy) within a given cohort. In some embodiments, the methods and systems described herein predict 70% or greater the subjects that are responders within a given cohort. In some embodiments, the methods and systems described herein predict 80% or greater the subjects that are responders within a given cohort. In some embodiments, the methods and systems described herein predict 90% or greater the subjects that are responders within a given cohort. In some embodiments, the methods and systems described herein predict 100% the subjects that are responders within a given cohort. In some embodiments, the methods and systems described herein predict 65% or greater the subjects that are non-responders (e.g., will not respond to anti-TNF therapy) within a given cohort. In some embodiments, the methods and systems described herein predict 70% or greater the subjects that are non-responders within a given cohort. In some embodiments, the methods and systems described herein predict 80% or greater the subjects that are non-responders within a given cohort. In some embodiments, the methods and systems described herein predict 90% or greater the subjects that are non-responders within a given cohort. In some embodiments, the methods and systems described herein predict 100% of the subjects that are non-responders within a given cohort.

In some embodiments, a gene expression response signature is developed by assessing one or more genes comprising: ETV1, IL13RA2, PDPN, KATNAL1, LOC100505918, CXCL2, SIRT4, RPRDIA, DMD, PDLIM4, AKAP12, ABTB1, IL7R, ZC4H2, RNF24, GOLGA6L6, TOLLIP, DLX5, FAM86C1, SEZ6L, SOD2, SOD2-OT1, SSR4P1, ABHD12, GPR161, DRAM1, TNC, H2BC3, MPI, MMP10, VASHI, LINC01241, C16orf58, ZNF510, RASSF9, MEIS1, RHOJ, USP54, INHBA, PPMIA, NAAA, NFE2L1, DALRD3, LOC101929243, PSG9, RAP2C, TMEM158, TRDV2, YMEILI, TRAC, TRAJ17, TRAV20, ADGRL2, LIMS1, LIMS4, OPN1SW, TAL1, N4BP2L1, PROX1-AS1, RBM48, TSPAN2, PTK2B, OTX1, PRKAR2B, ADAMTS12, SNX29, ADAMTS17, DKK3, ABCC5, STC1, SNAPCI, MS4A7, SRPK3, CXCL6, IL11, CEBPB, SLC25A29, SGK2, SPACA9, MMP3, RPUSD3, CXCL1, IL411, FRMD6, SPART, BBOX1, PAX5, RBPJ, WNT5A, AP2A2, TRAF1, PLG, ZEB2, PLAU, AMIGO2, EPS15L1, KLHL6, NRCAM, MGAT4B, MAP3K20, TAGAP, SEC63, ASB10, SLC35G2, MMP12, LARP4B, AJUBA, SPIRE2, ARMCX2, PPP2R5C, ZMYND12, DOK4, GART, PIWIL4, SPPL3, CYLD, SELENBP1, KLHL5, ERO1B, RNF144B, C11orf96, BAD, PRR29, LRRFIP2, ZNF57, LINC02805, TRIM8, PEX26, CANX, POLR2C, PCBP1-AS1, MKRN1, NBN, IFIT3, LOC101929356, ARL5A, DZIP1, PYM1, GNA11, PXMP4, SIAH2, DNAJC27, SPOCK1, SAMSN1, SRGN, TOR1AIP1, HOOK2, APLP2, LINC01159, RBMSI, RAB23, WDR47, ACSS2, CHN2, TRIM3, LINC00888, TM7SF3, IGSF3, C2orf88, PHBP19, MLKL, HADH, C3orf62, PDZD3, CD86, PXDN, RHBDD1, S100A9, HDGFL3, KLHL12, IER5, IGFBP5, LRRC8C, NR3C1, RGS5, PTGFR, TFPI2, HGF, PAPPA, or CFLAR.

In some embodiments, a gene expression response signature is developed by assessing one or more genes comprising: GOS2, ARHGAP18, HCAR3, GABARAPL1, SF3B2, LILRA3, TLR2, APOBEC3A_B, APOBEC3A, MRPS16, GK3P, WNK2, TFPI, SLC7A8, SUPV3L1, CLEC4E, TREM1, C5AR1, CDCA7, PLK4, TARDBP, CNTN3, MLN, ECH1, CDCA7L, ECSIT, CEMIP, LOC254896, CMTM2, OLRI, RASGRP4, NKAPL, ACOT9, HNRNPA3, ZWINT, SLC22A4, FCGR3B, CXCL8, ARL11, CXCR1, PROK2, SOD2, SOD2-OT1, IFITM2, IL11, MRPLI, ZBED3, DGAT2, KIFC1, DUSPI, WNT5A, FCN1, DUT, PI15, TAGAP, NRIP3, PDE4B, FGR, MASP1, RIPK2, CCNB1, PLEK, LILRA6, ELOVL6, UBR7, GLTIDI, GLA, LATS2, MIR3945HG, STEAP4, CHTOP, TLR4, BAGS, RUVBL2, ASPHDI, TIMM23B, TIMM23, FGF2, GLG1, MNDA, TBCE, FAM98B, IDH3B, PILRA, BCL2A1, MRPL12, PAPPA, CD82, SKA2, BMP1, GNAI1, MAP4K4, LILRA5, MEFV, SSRP1, HAPLNI, AQP9, PPARGCIB, RGS2, CYP4F3, TSN, NUDT19, CHEK1, GNS, GATD3A, SMIM25, UPPI, SET, SETSIP, LSM5, FCGRICP, FCGRIB, MGAM, ABL2, CLEC4D, LILRA2, GALNT15, FFAR2, ATG4B, B3GNTL1, SLC11A1, FST, NFIL3, RPIA, SMC2, IFIT2, NINJ1, DYRKIA, SERTAD4-AS1, IRAK2, TOMM70, NUP88, PCDH20, LINC02339, HGF, SHMTI, MIR6778, CTDSPL, S100A12, RAB15, GFPT2, CHST10, PFKFB3, STC1, ADGRG3, FPR2, IL1B, GADD45B, IL1RN, TNFRSF11B, NAMPT, PTGS2, TNFAIP6, CSF3R, CREB5, IL17RB, SNCA, BCL6, GK, TNFRSF10C, ACSL1, OSM, FCGR1A|FCGR1B, FPR1, SRGN, FCGR3B|FCGR3A, TMEM97, KCNJ15, RNF13, or CXCL11.

In some embodiments, a gene expression response signature is developed by assessing one or more genes comprising: ABCC5, ABHD12, ABTB1, ADAMTS12, AJUBA, AMIGO2, AP2A2, BAD, C16orf58, CANX, CEBPB, CFLAR, CHN2, CXCL1, CXCL2, CXCL6, DRAM1, EPS15L1, FAM86C1, H2BC3, HGF, IFIT3, IGFBP5, IL13RA2, IL7R, INHBA, KLHL12, LARP4B, LIMS1, LRRC8C, MAP3K20, MEIS1, MMP12, MS4A7, NAAA, NBN, NFE2L1, NR3C1, OTX1, PAPPA, PAX5, PLAU, PLG, PPMIA, PTK2B, RGS5, RHBDD1, RNF144B, S100A9, SIAH2, SLC25A29, SLC35G2, SNX29, SOD2, SPIRE2, SPPL3, STC1, TAL1, TNC, TOLLIP, TOR1AIP1, TRAF1, TRDV2, TRIM8, USP54, or ZNF57.

In some embodiments, a gene expression response signature is developed by assessing one or more genes comprising: ABL2, AQP9, ATG4B, BAG5, BCL2A1, BCL6, BMP1, C5AR1, CCNB1, CD82, CDCA7L, CHEK1, CHST10, CHTOP, CLEC4E, CREB5, CSF3R, CXCL11, CXCL8, CXCR1, CYP4F3, DUSP1, DYRK1A, ECH1, ECSIT, FCGR1B, FCGR1CP, FCGR3B, FCGR3B|FCGR3A, FCN1, FFAR2, FGF2, FGR, FPR1, FPR2, FST, GABARAPL1, GALNT15, GK, GK3P, GNAI1, HGF, IDH3B, IFIT2, ILIB, IRAK2, KIFC1, LATS2, MASP1, MEFV, MNDA, NFIL3, NINJ1, NUP88, PAPPA, PI15, PLEK, PTGS2, RIPK2, RPIA, RUVBL2, SET, SLC22A4, SLC7A8, SMC2, SNCA, SOD2, SSRP1, STC1, SUPV3L1, TARDBP, TLR2, TLR4, TMEM97, TNFAIP6, TREM1, or TSN.

In some embodiments, a gene expression response signature is developed by assessing one or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPMIA, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPL1, TMEM97, TLR2, FPR2, IL1B, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGR1CP, FCGR1B, ABL2, GALNT15, FFAR2, ATG4B, FST, NFIL3, RPIA, SMC2, IFIT2, NINJ1, DYRK1A, IRAK2, NUP88, or CHST10.

In some embodiments, a gene expression response signature is developed by assessing SOD2, PAPPA, HGF, or STC1.

In some embodiments, a gene expression response signature is developed by assessing AMIGO2, CEBPB, CXCL1, CXCL2, CXCL6, DRAM1, IGFBP5, MAP3K20, MEIS1, MMP12, MS4A7, or NR3C1.

### Defining Classifier(s)

A provided gene expression response signature is a gene or set of genes that can be used to determine whether a subject will or will not respond to a particular therapy (e.g., anti-TNF therapy). A gene expression response signature itself can be a classifier, or can otherwise be part of a classifier that distinguishes between responsive and non-responsive subjects. In some embodiments, a gene expression response signature can be identified using mRNA or protein expression datasets, for example as may be or have been prepared from validated biological data (e.g., biological data derived from publicly available databases such as Gene Expression Omnibus (“GEO”)). In some embodiments, a gene expression response signature may be derived by comparing gene expression levels of known responsive and known non-responsive prior subjects to a specific therapy (e.g., anti-TNF therapy). In some embodiments, certain genes (e.g., signature genes) are selected from this cohort of gene expression data to be used in developing the gene expression response signature.

In some embodiments, signature genes are identified by methods analogous to those reported by Santolini, “A personalized, multiomics approach identifies genes involved in cardiac hypertrophy and heart failure,” Systems Biology and Applications, (2018) 4:12; doi: 10.1038/s41540-018-0046-3, which is incorporated herein by reference for all purposes. In some embodiments, signature genes are identified by comparing gene expression levels of known responsive and non-responsive prior subjects and identifying significant changes between the two groups, wherein the significant changes can be large differences in expression (e.g., greater than 2-fold change), small differences in expression (e.g., less than 2-fold change), or both. In some embodiments, genes are ranked by significance of difference in expression. In some embodiments, significance is measured by Pearson correlation between gene expression and response outcome. In some embodiments, signature genes are selected from the ranking by significance of difference in expression. In some embodiments, the number of signature genes selected is less than the total number of genes analyzed. In some embodiments, 200 signature genes or less are selected. In some embodiments 100 genes or less are selected.

In some embodiments, signature genes are selected in conjunction with their location on a human interactome (HI), a map of protein-protein interactions. Use of the HI in this way encompasses a recognition that mRNA activity is dynamic and determines the actual over and under expression of proteins critical to understanding certain diseases. In some embodiments, genes associated with response to certain therapies (e.g., anti-TNF therapy) may cluster (e.g., form a cluster of genes) in discrete modules on the HI map. The existence of such clusters is associated with the existence of fundamental underlying disease biology. In some embodiments, a gene expression response signature is derived from signature genes selected from the cluster of genes on the HI map. Accordingly, in some embodiments, a gene expression response signature is derived from a cluster of genes associated with response to anti-TNF therapy on a human interactome map.

In some embodiments, genes associated with response to certain therapies exhibit certain topological properties when mapped onto a human interactome map. For example, in some embodiments, a plurality of genes associated with response to anti-TNF therapy and characterized by their position (e.g., topological properties, e.g., their proximity to one another) on a human interactome map.

In some embodiments, genes associated with response to certain therapies (e.g., anti-TNF therapy) may exist within close proximity to one another on the HI map. Said proximal genes, do not necessarily share fundamental underlying disease biology. That is, in some embodiments, proximal genes do not share significant protein interaction. Accordingly, in some embodiments, the gene expression response signature is derived from genes that are proximal on a human interactome map. In some embodiments, the gene expression response signature is derived from certain other topological features on a human interactome map.

In some embodiments, genes associated with response to certain therapies (e.g., anti-TNF therapy) may be determined by Diffusion State Distance (DSD) (see Cao, et al. PLOS One, 8 (10): e76339 (Oct. 23, 2013), which is incorporated herein by reference for all purposes) when used in combination with the HI map.

In some embodiments, signature genes are selected by (1) ranking genes based on the significance of difference of expression of genes as compared to known responders and known non-responders; (2) selecting genes from the ranked genes and mapping the selected genes onto a human interactome map; and (3) selecting signature genes from the genes mapped onto the human interactome map.

In some embodiments, signature genes (e.g., selected from the Santolini method, or using various network topological properties including, but not limited to, clustering, proximity and diffusion-based methods) are provided to a probabilistic neural network or other classifier described herein to thereby provide (e.g., “train”) the gene expression response signature. In some embodiments, the probabilistic neural network implements the algorithm proposed by D. F. Specht in “Probabilistic Neural Networks,” Neural Networks, 3 (1): 109-118 (1990), which is incorporated herein by reference for all purposes. In some embodiments, the probabilistic neural network is written in the R-statistical language, and knowing a set of observations described by a vector of quantitative variables, classifies observations into a given number of groups (e.g., responders and non-responders). The algorithm is trained with the data set of signature genes taken from known responders and non-responders and provides new observations. In some embodiments, the probabilistic neural network is one derived from the Comprehensive R Network.

Alternatively or additionally, in some embodiments, a gene expression response signature can be trained in the probabilistic neural network using a cohort of known responders and non-responders using leave-one-out cross or k-fold cross validation. In some embodiments, such a process leaves one sample out (e.g., leave-one-out) of the analysis and trains the classifier based on the remaining samples. In some embodiments, the updated classifier is then used to predict a probability of response for the sample that's left out. In some embodiments, such a process can be repeated iteratively, for example, until all samples have been left out once. In some embodiments, such a process randomly partitions a cohort of known responders and non-responders into k equal sizes groups. Of the k groups, a single group is retained as validation data for testing the model, and the remaining groups are used as training data. Such a process can be repeated k times, with each of the k groups being used exactly once as the validation data. In some embodiments, the outcome is a probability score for each sample in the training set. Such probability scores can correlate with actual response outcome. A Recursive Operating Curves (ROC) can be used to estimate the performance of the classifier. In some embodiments, an Area Under Curve (AUC) of about 0.6 or higher reflects a suitably validated classifier. In some embodiments, a Negative Predictive Value (NPV) of 0.9 reflects a suitable validated classifier. In some embodiments, a Positive Predictive Value (PPV) of 0.9 reflects a suitable validated classifier. In some embodiments, a classifier can be tested in a completely independent (e.g., blinded) cohort to, for example, confirm the suitability (e.g., using leave-one-out or k-fold cross validation). Accordingly, in some embodiments, provided methods further comprise validating a gene expression response signature, for example, by assigning probability of response to a group of known responders and non-responders; and checking the gene expression response signature against a blinded group of responders and non-responders. The output of these processes is a trained gene expression response signature useful for establishing whether a subject will or will not respond to a particular therapy (e.g., anti-TNF therapy).

In some embodiments, a gene expression response signature is validated using a cohort of subjects having previously been treated with anti-TNF therapy, but is independent from the cohort of subjects used to prepare the classifier. In some embodiments, a gene expression response signature is considered “validated” when 90% or greater of non-responding subjects are predicted with 50% or greater accuracy within the validating cohort.

In some embodiments, the gene expression response signature predicts responsiveness of subjects with at least 50% accuracy across a population of subjects. In some embodiments, the gene expression response signature predicts responsiveness of subjects with at least 60% accuracy predicting responsiveness across a population of subjects. In some embodiments, the gene expression response signature predicts responsiveness of subjects with at least 80% accuracy across a population of subjects. In some embodiments, the gene expression response signature predicts responsiveness of subjects with at least 90% accuracy across a population of subjects. In some embodiments, the gene expression response signature predicts responsiveness of subjects with at least 95% accuracy across a population of subjects. In some embodiments, the gene expression response signature predicts responsiveness of subjects with at least 97% accuracy across a population of subjects. In some embodiments, the gene expression response signature predicts responsiveness of subjects with at least 98% accuracy across a population of subjects. In some embodiments, the gene expression response signature predicts responsiveness of subjects with at least 99% accuracy across a population of subjects.

Accordingly, in some embodiments, the gene expression response signature is established to distinguish between responsive and non-responsive prior subjects who have received a type of therapy, e.g., anti-TNF therapy. This gene expression response signature, derived from these prior responders and non-responders, is used to classify subjects (outside of the previously-identify cohorts) as responders or non-responders, e.g., can predict whether a subject will or will not respond to a given therapy. In some embodiments, the response and non-responsive prior subjects suffered from the same disease, disorder, or condition.

In some embodiments, a classifier is validated by analyzing gene expression levels in biological samples from a first cohort of subjects who have previously received the anti-TNF therapy (“prior subjects”) and have been determined to respond (“responders”) or not to respond (“non-responders”) to the anti-TNF therapy to identify genes that show statistically significant differences in expression level between the responders and the non-responders (“signature genes”). In some embodiments, signature genes are mapped onto a biological network (e.g., a human interactome). In some embodiments, a subset of signature genes are selected on the basis of their connectivity in the biological network to provide a candidate gene list. In some embodiments, a method of validating a classifier comprising training a classifier (e.g., an non-validated classifier) on expression levels of the genes of the candidate gene list from the first cohort of subjects (e.g, prior subjects, that is, subjects who have previously been classified as responsive or non-responsive to anti-TNF therapy) to identify a subset of the prior subjects having a pattern of expression of the candidate gene list indicative that the subset of prior subjects are unlikely to respond to the anti-TNF therapy, to thereby obtain a trained classifier.

In some embodiments, a trained classifier is validated via analysis of a second cohort comprising an independent and blinded group of responders and non-responders, and selecting a cutoff score such that the validated classifier distinguishes about 50% of prior subjects that are non-responsive (e.g., have a TNR of about 0.5 or have a TPR of about 0.5) to the anti-TNF therapy. In some embodiments, a validated classifier distinguishes about 65% of prior subjects that are non-responsive (e.g., have a TNR of about 0.65 or have a TPR of about 0.65) to the anti-TNF therapy. In some embodiments, a validated classifier distinguishes about 70% of prior subjects that are non-responsive (e.g., have a TNR of about 0.7 or have a TPR of about 0.7) to the anti-TNF therapy. In some embodiments, a validated classifier distinguishes about 80% of prior subjects that are non-responsive (e.g., have a TNR of about 0.8 or have a TPR of about 0.8) to the anti-TNF therapy. In some embodiments, a validated classifier distinguishes about 90% of prior subjects that are non-responsive (e.g., have a TNR of about 0.9 or have a TPR of about 0.9) to the anti-TNF therapy. In some embodiments, a validated classifier distinguishes about 95% of prior subjects that are non-responsive (e.g., have a TNR of about 0.95 or have a TPR of about 0.95) to the anti-TNF therapy. In some embodiments, a validated classifier distinguishes about 100% of prior subjects that are non-responsive (e.g., have a TNR of about 1.0 or have a TPR of about 1.0) to the anti-TNF therapy. As described elsewhere herein, positive predictive value (PPV) and true positive rate (TPR) can also be associated with or determined from combinations of true negative rate (TNR), negative predictive value (NPV), false positive rate, false negative rate, sensitivity, and specificity

In some embodiments, a validated classifier distinguishes at least 50% of prior subjects that are non-responsive to the anti-TNF therapy with at least 60% NPV or 60% PPV (e.g., has an NPV of about 0.6 or has a PPV of about 0.6). In some embodiments, a validated classifier distinguishes at least 50% of prior subjects that are non-responsive to the anti-TNF therapy with at least 70% NPV or 70% PPV (e.g., has an NPV of about 0.7 or has a PPV of about 0.7). In some embodiments, a validated classifier distinguishes at least 50% of prior subjects that are non-responsive to the anti-TNF therapy with at least 80% NPV or 80% PPV (e.g., has an NPV of about 0.8 or has a PPV of about 0.8). In some embodiments, a validated classifier distinguishes at least 50% of prior subjects that are non-responsive to the anti-TNF therapy with at least 90% NPV or 90% PPV (e.g., has an NPV of about 0.9 or has a PPV of about 0.9). In some embodiments, a validated classifier distinguishes at least 50% of prior subjects that are non-responsive to the anti-TNF therapy with at least 95% NPV or 95% PPV (e.g., has an NPV of about 0.95 or has a PPV of about 0.95). In some embodiments, a validated classifier distinguishes at least 50% of prior subjects that are non-responsive to the anti-TNF therapy with at least 100% NPV or 100% PPV (e.g., has an NPV of about 1.0 or has a PPV of about 1.0). As described elsewhere positive predictive value (PPV) and true positive rate (TPR) can also be associated with or determined from combinations of true negative rate (TNR), negative predictive value (NPV), false positive rate, false negative rate, sensitivity, and specificity.

Many statistical classification techniques are suitable as approaches to perform the classification described above (e.g., distinguish between subjects who do and who do not respond to anti-TNF therapy). Such methods include but are not limited to supervised learning approaches.

In supervised learning approaches, a group of samples from two or more groups (e.g., those do and do not respond to anti-TNF therapy) are analyzed or processed with a statistical classification method. Absence/presence of genes or particular SNPs or variants, or expression level of genes or biomarkers described herein can be used as a basis for classifier that differentiates between the two or more groups. A new sample can then be analyzed or processed so that the classifier can associate the new sample with one of the two or more groups.

Commonly used supervised classifiers include without limitation the neural network (e.g, artificial neural network, multi-layer perceptron), support vector machines, k-nearest neighbors, Gaussian mixture model, Gaussian, naive Bayes, decision tree and radial basis function (RBF) classifiers. Linear classification methods include Fisher's linear discriminant, logistic regression, naive Bayes classifier, perceptron, and support vector machines (SVMs). Other classifiers for use with methods according to the disclosure include quadratic classifiers, k-nearest neighbor, boosting, decision trees, random forests, neural networks, pattern recognition, Bayesian networks and Hidden Markov models. Other classifiers, including improvements or combinations of any of these, commonly used for supervised learning, can also be suitable for use with the methods described herein.

Classification using supervised methods can generally be performed by the following methodology:
1. Gather a training set. These can include, for example, expression levels of one or more genes or biomarkers described herein from a sample from a patient responding or not responding to anti-TNF therapy. The training samples are used to “train” the classifier.
2. Determine the input “feature” representation of the learned function. The accuracy of the learned function depends on how the input object is represented. Typically, the input object is transformed into a feature vector, which contains a number of features that are descriptive of the object. The features might include a set of genes detected in a sample from a patient or subject.
3. Determine the structure of the learned function and corresponding learning algorithm. A learning algorithm is chosen, e.g., artificial neural networks, decision trees, Bayes classifiers or support vector machines. The learning algorithm is used to build the classifier.
4. Build the classifier (e.g., classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set. The built model can involve feature coefficients or importance measures assigned to individual features.

In some cases, the individual features are individual genes or levels of individual genes. In some cases, the level of the gene is a normalized value, an average value, a median value, a mean value, an adjusted average, or other adjusted level or value. The individual features may comprise or consist of sets or panels of genes, such as the sets provided herein.

Once the classifier (e.g., classification model) is determined as described above (“trained”), it can be used to classify a sample e.g., a patient sample comprising expressed genes that is analyzed or processed according to methods described herein.

### Detecting Classifier(s)

Detecting gene classifiers in subjects, once the gene classifier is identified, is a method. In other words, by first defining the gene classifier, a variety of methods can be used to determine whether a subject or group of subjects express the established gene classifier. For example, in some embodiments, a practitioner can obtain a blood or tissue sample from the subject prior to administering of therapy, and extract and analyzemRNA profiles from said blood or tissue sample. The analysis of gene expression profiles can be performed by any method appropriate to those practicing the present disclosure, including, but not limited hybridization-based RNA detection assays (such as assays based on microarray, bead array, and NANOSTRING (direct detection of color-coded hybridized probes) technologies), RNA sequencing assays, amplification-based RNA detection assays (such as real-time quantitative reverse transcription polymerase chain reaction (qRT-PCR) or reverse transcription loop mediated isothermal amplification (RT-LAMP)), mass spectrometry-based protein detection assays (such as targeted mass spectrometry (MRM or SRM) or immunoaffinity liquid chromatography-tandem mass spectrometry (IA LC-MS/MC)) and immunoassay-based protein detection assays (such as enzyme-linked immunosorbent assays (ELISA), immunohistochemistry, or flow cytometry). Accordingly, in some embodiments, the present disclosure provides methods of determining whether a subject is classified as a responder or non-responder, comprising measuring gene expression by at least one of a microarray, RNA sequencing, real-time quantitative reverse transcription PCR (qRT-PCR), bead array, and ELISA. In some embodiments, the present disclosure provides methods of determining whether a subject is classified as a responder or non-responder comprising measuring gene expression of a subject by RNA sequencing (e.g., RNAseq).

In some embodiments, the provided technologies provide methods comprising determining, prior to administering anti-TNF therapy, that a subject displays a gene expression response signature associated with response to anti-TNF therapy; and administering the anti-TNF therapy to the subject determined to display the gene expression response signature. In some embodiments, the provided technologies provide methods comprising determining, prior to administering anti-TNF therapy, that a subject does not display the gene expression response signature; and administering a therapy alternative to anti-TNF therapy to the subject determine not to display the gene expression signature.

In some embodiments, the therapy alternative to anti-TNF therapy comprises rituximab (Rituxan®), sarilumab (Kevzara®), tofacitinib citrate (Xeljanz®), leflunomide (Arava®), vedolizumab (Entyvio®), tocilizumab (Actemra®), anakinra (Kineret®), abatacept (Orencia®), or a combination thereof.

In some embodiments, gene expression is measured by subtracting background data, correcting for batch effects, and dividing by mean expression of housekeeping genes. See Eisenberg & Levanon, “Human housekeeping genes, revisited,” Trends in Genetics, 29 (10): 569-574 (October 2013), which is incorporated herein by reference for all purposes. In the context of microarray data analysis, background subtraction refers to subtracting the average fluorescent signal arising from probe features on a chip not complimentary to any mRNA sequence, e.g, signals that arise from non-specific binding, from the fluorescence signal intensity of each probe feature. The background subtraction can be performed with different software packages, such as Affymetrix® Gene Expression Console. Housekeeping genes are involved in basic cell maintenance and, therefore, are expected to maintain constant expression levels in all cells and conditions. The expression level of genes of interest, e.g., those in the response signature, can be normalized by dividing the expression level by the average expression level across a group of selected housekeeping genes. This housekeeping gene normalization procedure calibrates the gene expression level for experimental variability. Further, normalization methods such as robust multi-array average (“RMA”) correct for variability across different batches of microarrays, are available in R packages recommended by either Illumina® or Affymetrix® platforms. The normalized data is log transformed, and probes with low detection rates across samples are removed. Furthermore, probes with no available genes symbol or Entrez ID are removed from the analysis.

In some embodiments, the present disclosure provides a kit comprising mechanisms for detecting a gene expression response signature established to distinguish between responsive and non-responsive prior subjects who have received anti-TNF therapy. In some embodiments, the kit facilitates comparison levels of gene expression of a subject to the gene expression response signature (e.g., the gene classifier) established to distinguish between responsive and non-responsive prior subjects who have received anti-TNF therapy. In some embodiments, a kit comprises a set of reagents for detecting an expression level of one or more genes in a gene expression response signature described herein.

In some embodiments, the present disclosure provides a kit comprising mechanisms for detecting a gene expression response signature established to distinguish between responsive and non-responsive prior subjects suffering from a disease, disorder, or condition and who have received anti-TNF therapy, wherein the gene expression response signature comprises an expression level of SOD2, PAPPA, HGF, or STC1.

In some embodiments, the present disclosure provides a kit for evaluating a likelihood that a patient having an autoimmune disorder will not respond to an anti-TNF therapy, the kit comprising a set of reagents for detecting an expression level of one or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPM1A, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPL1, TMEM97, TLR2, FPR2, IL1B, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGR1CP, FCGR1B, ABL2, GALNT15, FFAR2, ATG4B, FST, NFIL3, RPIA, SMC2, IFIT2, NINJ1, DYRK1A, IRAK2, NUP88, or CHST10.

As described herein, a kit comprises a set of reagents for detecting or measuring expression level of one or more genes described herein. In some embodiments, a kit comprises components for hybridization-based RNA detection assays (such as assays based on microarray, bead array, and NANOSTRING (direct detection of color-coded hybridized probes) technologies), RNA sequencing assays, amplification-based RNA detection assays (such as real-time quantitative reverse transcription polymerase chain reaction (qRT-PCR) or reverse transcription loop mediated isothermal amplification (RT-LAMP)), mass spectrometry-based protein detection assays (such as targeted mass spectrometry (MRM or SRM) or immunoaffinity liquid chromatography-tandem mass spectrometry (IA LC-MS/MC)) and immunoassay-based protein detection assays (such as enzyme-linked immunosorbent assays (ELISA), immunohistochemistry, or flow cytometry).

In some embodiments, the gene expression response signature comprises an expression level of (1) SOD2, PAPPA, HGF, or STC1 and (2) one or more genes comprising: ABCC5, ABHD12, ABTB1, ADAMTS12, AJUBA, AMIGO2, AP2A2, BAD, C16orf58, CANX, CEBPB, CFLAR, CHN2, CXCL1, CXCL2, CXCL6, DRAM1, EPS15L1, FAM86C1, H2BC3, HGF, IFIT3, IGFBP5, IL13RA2, IL7R, INHBA, KLHL12, LARP4B, LIMS1, LRRC8C, MAP3K20, MEIS1, MMP12, MS4A7, NAAA, NBN, NFE2L1, NR3C1, OTX1, PAPPA, PAX5, PLAU, PLG, PPM1A, PTK2B, RGS5, RHBDD1, RNF144B, S100A9, SIAH2, SLC25A29, SLC35G2, SNX29, SOD2, SPIRE2, SPPL3, STC1, TAL1, TNC, TOLLIP, TOR1AIP1, TRAF1, TRDV2, TRIM8, USP54, or ZNF57.

In some embodiments the gene expression response signature comprises an expression level of (1) SOD2, PAPPA, HGF, or STC1 and (2) one or more genes comprising: ABL2, AQP9, ATG4B, BAG5, BCL2A1, BCL6, BMP1, C5AR1, CCNB1, CD82, CDCA7L, CHEK1, CHST10, CHTOP, CLEC4E, CREB5, CSF3R, CXCL11, CXCL8, CXCR1, CYP4F3, DUSP1, DYRK1A, ECH1, ECSIT, FCGR1B, FCGR1CP, FCGR3B, FCGR3B|FCGR3A, FCN1, FFAR2, FGF2, FGR, FPR1, FPR2, FST, GABARAPL1, GALNT15, GK, GK3P, GNAI1, HGF, IDH3B, IFIT2, IL1B, IRAK2, KIFC1, LATS2, MASP1, MEFV, MNDA, NFIL3, NINJ1, NUP88, PAPPA, PI15, PLEK, PTGS2, RIPK2, RPIA, RUVBL2, SET, SLC22A4, SLC7A8, SMC2, SNCA, SOD2, SSRP1, STC1, SUPV3L1, TARDBP, TLR2, TLR4, TMEM97, TNFAIP6, TREM1, or TSN.

In some embodiments, the gene expression response signature comprises an expression level of (1) SOD2, PAPPA, HGF, or STC1 and (2) one or more genes comprising: ABCC5, ABHD12, ABTB1, ADAMTS12, AJUBA, AMIGO2, AP2A2, BAD, C16orf58, CANX, CEBPB, CFLAR, CHN2, CXCL1, CXCL2, CXCL6, DRAM1, EPS15L1, FAM86C1, H2BC3, HGF, IFIT3, IGFBP5, IL13RA2, IL7R, INHBA, KLHL12, LARP4B, LIMS1, LRRC8C, MAP3K20, MEIS1, MMP12, MS4A7, NAAA, NBN, NFE2L1, NR3C1, OTX1, PAPPA, PAX5, PLAU, PLG, PPM1A, PTK2B, RGS5, RHBDD1, RNF144B, S100A9, SIAH2, SLC25A29, SLC35G2, SNX29, SOD2, SPIRE2, SPPL3, STC1, TAL1, TNC, TOLLIP, TOR1AIP1, TRAF1, TRDV2, TRIM8, USP54, or ZNF57.

### Using Classifiers

### Patient Stratification

Among other things, the present disclosure provides technologies for predicting responsiveness to anti-TNF therapies. In some embodiments, provided technologies exhibit consistency or accuracy across cohorts superior to other methodologies.

Thus, the present disclosure provides technologies for patient stratification, defining or distinguishing between responder and non-responder populations. For example, in some embodiments, the present disclosure provides methods for treating subjects with anti-TNF therapy, which methods, in some embodiments, comprise: administering the anti-TNF therapy to subjects who have been determined not to display a gene expression response signature established to distinguish between responsive and non-responsive prior subjects who have received the anti-TNF therapy. In some such embodiments, the gene expression response signature includes a plurality of genes established to distinguish between responsive and non-responsive prior subjects for a given anti-TNF therapy. In some embodiments, the plurality of genes are determined to cluster with one another in a human interactome map. In some embodiments, the plurality of genes are proximal in a human interactome map. In some embodiments, the plurality of genes comprise genes that are shown to be statistically significantly different between responsive and non-responsive prior subjects.

### Methods of Treatment and Therapy Monitoring

Further, the present disclosure provides technologies for monitoring therapy for a given subject or cohort of subjects. As a subject's gene expression level can change over time, it may, in some instances, be desirable to evaluate a subject at one or more points in time, for example, at specified and or periodic intervals.

In some embodiments, the present disclosure provides a method of treating a subject suffering from a disease, disorder, or condition (e.g., inflammatory bowel disease, ulcerative colitis or Crohn's disease) with an anti-TNF therapy, the method comprising: administering the anti-TNF therapy to subjects who have been determined not to display a gene expression response signature established to distinguish between responsive and non-responsive prior subjects who have received the anti-TNF therapy, wherein the gene expression response signature comprises an expression level of SOD2, PAPPA, HGF, or STC1.

In some embodiments, the present disclosure provides a method of treating a subject suffering from a disease, disorder, or condition with an anti-TNF therapy, the method comprising: administering the anti-TNF therapy to subjects who have been determined to be responsive via a classifier determined to distinguish between responsive and non-responsive subjects who have received the anti-TNF therapy (“prior subject”), and the classifier measures expression of one or more genes (e.g., two or more, three or more, four or more, five or more, six or more, or substantially all) comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPM1A, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPL1, TMEM97, TLR2, FPR2, ILIB, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGR1CP, FCGR1B, ABL2, GALNT15, FFAR2, ATG4B, FST, NFIL3, RPIA, SMC2, IFIT2, NINJ1, DYRK1A, IRAK2, NUP88, or CHST10.

In some embodiments, the classifier measures expression of one or more genes (e.g., two or more, three or more, four or more, five or more, six or more, or substantially all) comprising: ABCC5, ABHD12, ABTB1, ADAMTS12, AJUBA, AMIGO2, AP2A2, BAD, C16orf58, CANX, CEBPB, CFLAR, CHN2, CXCL1, CXCL2, CXCL6, DRAM1, EPS15L1, FAM86C1, H2BC3, HGF, IFIT3, IGFBP5, IL13RA2, IL7R, INHBA, KLHL12, LARP4B, LIMS1, LRRC8C, MAP3K20, MEIS1, MMP12, MS4A7, NAAA, NBN, NFE2L1, NR3C1, OTX1, PAPPA, PAX5, PLAU, PLG, PPM1A, PTK2B, RGS5, RHBDD1, RNF144B, S100A9, SIAH2, SLC25A29, SLC35G2, SNX29, SOD2, SPIRE2, SPPL3, STC1, TAL1, TNC, TOLLIP, TOR1AIP1, TRAF1, TRDV2, TRIM8, USP54, or ZNF57.

In some embodiments, the classifier measures expression of one or more genes (e.g., two or more, three or more, four or more, five or more, six or more, or substantially all) comprising: ABL2, AQP9, ATG4B, BAG5, BCL2A1, BCL6, BMP1, C5AR1, CCNB1, CD82, CDCA7L, CHEK1, CHST10, CHTOP, CLEC4E, CREB5, CSF3R, CXCL11, CXCL8, CXCR1, CYP4F3, DUSP1, DYRK1A, ECH1, ECSIT, FCGR1B, FCGR1CP, FCGR3B, FCGR3B|FCGR3A, FCN1, FFAR2, FGF2, FGR, FPR1, FPR2, FST, GABARAPL1, GALNT15, GK, GK3P, GNAI1, HGF, IDH3B, IFIT2, ILIB, IRAK2, KIFC1, LATS2, MASP1, MEFV, MNDA, NFIL3, NINJ1, NUP88, PAPPA, PI15, PLEK, PTGS2, RIPK2, RPIA, RUVBL2, SET, SLC22A4, SLC7A8, SMC2, SNCA, SOD2, SSRP1, STC1, SUPV3L1, TARDBP, TLR2, TLR4, TMEM97, TNFAIP6, TREM1, or TSN.

In some embodiments, the classifier measures expression of SOD2, PAPPA, HGF, or STC1.

In some embodiments, the classifier measures expression of one or more genes (e.g., two or more, three or more, four or more, five or more, six or more, or substantially all) comprising: AMIGO2, CEBPB, CXCL1, CXCL2, CXCL6, DRAM1, IGFBP5, MAP3K20, MEIS1, MMP12, MS4A7, or NR3C1.

In some embodiments, the classifier measures expression levels of two or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPM1A, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPL1, TMEM97, TLR2, FPR2, ILIB, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGR1CP, FCGR1B, ABL2, GALNT15, FFAR2, ATG4B, FST, NFIL3, RPIA, SMC2, IFIT2, NINJ1, DYRK1A, IRAK2, NUP88, or CHST10.

In some embodiments, a gene expression response signature comprises an expression level of (1) SOD2, PAPPA, HGF, or STC1 and (2) one or more genes comprising: ABCC5, ABHD12, ABTB1, ADAMTS12, AJUBA, AMIGO2, AP2A2, BAD, C16orf58, CANX, CEBPB, CFLAR, CHN2, CXCL1, CXCL2, CXCL6, DRAM1, EPS15L1, FAM86C1, H2BC3, HGF, IFIT3, IGFBP5, IL13RA2, IL7R, INHBA, KLHL12, LARP4B, LIMS1, LRRC8C, MAP3K20, MEIS1, MMP12, MS4A7, NAAA, NBN, NFE2L1, NR3C1, OTX1, PAPPA, PAX5, PLAU, PLG, PPM1A, PTK2B, RGS5, RHBDD1, RNF144B, S100A9, SIAH2, SLC25A29, SLC35G2, SNX29, SOD2, SPIRE2, SPPL3, STC1, TAL1, TNC, TOLLIP, TOR1AIP1, TRAF1, TRDV2, TRIM8, USP54, or ZNF57.

In some embodiments, a gene expression response signature comprises an expression level of (1) SOD2, PAPPA, HGF, or STC1 and (2) one or more genes comprising: ABL2, AQP9, ATG4B, BAG5, BCL2A1, BCL6, BMP1, C5AR1, CCNB1, CD82, CDCA7L, CHEK1, CHST10, CHTOP, CLEC4E, CREB5, CSF3R, CXCL11, CXCL8, CXCR1, CYP4F3, DUSP1, DYRK1A, ECH1, ECSIT, FCGR1B, FCGR1CP, FCGR3B, FCGR3B|FCGR3A, FCN1, FFAR2, FGF2, FGR, FPR1, FPR2, FST, GABARAPL1, GALNT15, GK, GK3P, GNAI1, HGF, IDH3B, IFIT2, ILIB, IRAK2, KIFC1, LATS2, MASP1, MEFV, MNDA, NFIL3, NINJ1, NUP88, PAPPA, PI15, PLEK, PTGS2, RIPK2, RPIA, RUVBL2, SET, SLC22A4, SLC7A8, SMC2, SNCA, SOD2, SSRP1, STC1, SUPV3L1, TARDBP, TLR2, TLR4, TMEM97, TNFAIP6, TREM1, or TSN.

In some embodiments, the gene expression response signature comprises an expression level of (1) SOD2, PAPPA, HGF, or STC1 and (2) one or more genes comprising: ETV1, IL13RA2, PDPN, KATNAL1, LOC100505918, CXCL2, SIRT4, RPRD1A, DMD, PDLIM4, AKAP12, ABTB1, IL7R, ZC4H2, RNF24, GOLGA6L6, TOLLIP, DLX5, FAM86C1, SEZ6L, SOD2, SOD2-OT1, SSR4P1, ABHD12, GPR161, DRAM1, TNC, H2BC3, MPI, MMP10, VASH1, LINC01241, C16orf58, ZNF510, RASSF9, MEIS1, RHOJ, USP54, INHBA, PPM1A, NAAA, NFE2L1, DALRD3, LOC101929243, PSG9, RAP2C, TMEM158, TRDV2, YME1L1, TRAC, TRAJ17, TRAV20, ADGRL2, LIMS1, LIMS4, OPN1SW, TAL1, N4BP2L1, PROX1-AS1, RBM48, TSPAN2, PTK2B, OTX1, PRKAR2B, ADAMTS12, SNX29, ADAMTS17, DKK3, ABCC5, STC1, SNAPC1, MS4A7, SRPK3, CXCL6, IL11, CEBPB, SLC25A29, SGK2, SPACA9, MMP3, RPUSD3, CXCL1, IL411, FRMD6, SPART, BBOX1, PAX5, RBPJ, WNT5A, AP2A2, TRAF1, PLG, ZEB2, PLAU, AMIGO2, EPS15L1, KLHL6, NRCAM, MGAT4B, MAP3K20, TAGAP, SEC63, ASB10, SLC35G2, MMP12, LARP4B, AJUBA, SPIRE2, ARMCX2, PPP2R5C, ZMYND12, DOK4, GART, PIWIL4, SPPL3, CYLD, SELENBP1, KLHL5, ERO1B, RNF144B, C11orf96, BAD, PRR29, LRRFIP2, ZNF57, LINC02805, TRIM8, PEX26, CANX, POLR2C, PCBP1-AS1, or MKRN1.

In some embodiments, repeated monitoring under time permits or achieves detection of one or more changes in a subject's gene expression profile or characteristics that may impact ongoing treatment regimens. In some embodiments, a change is detected in response to which particular therapy administered to the subject is continued, is altered, or is suspended. In some embodiments, therapy may be altered, for example, by increasing or decreasing frequency or amount of administration of one or more agents or treatments with which the subject is already being treated. Alternatively or additionally, in some embodiments, therapy may be altered by addition of therapy with one or more new agents or treatments. In some embodiments, therapy may be altered by suspension or cessation of one or more particular agents or treatments.

To give but one example, if a subject is initially classified as responsive (because the subject's gene expression correlated to a gene expression response signature associated with a disease, disorder, or condition), a given anti-TNF therapy can then be administered. At a given interval (e.g., every six months, every year, etc.), the subject can be tested again to ensure that they still qualify as “responsive” to a given anti-TNF therapy. In the event the gene expression levels for a given subject change over time, and the subject no longer expresses genes associated with the gene expression response signature, or now expresses genes associated with non-responsiveness, the subject's therapy can be altered to suit the change in gene expression.

Accordingly, in some embodiments, the present disclosure provides methods of administering therapy to a subject previously determined not to display a gene expression response signature associated with anti-TNF therapy, wherein the subject does not display a gene expression response signature associated with response to anti-TNF therapy.

In some embodiments, the present disclosure provides methods of treating subjects with anti-TNF therapy, the method comprising: administering the anti-TNF therapy to subjects who have been determined not to display a gene expression response signature established to distinguish between responsive and non-responsive prior subjects who have received the anti-TNF therapy.

In some embodiments, the present disclosure provides methods further comprising determining, prior to the administering, that a subject does not display the gene expression response signature; and administering the anti-TNF therapy to the subject determined not to display the gene expression response signature.

In some embodiments, the present disclosure provides methods further comprising determining, prior to the administering, that a subject does display the gene expression response signature; and administering a therapy alternative to anti-TNF therapy to the subject determined to display the gene expression response signature.

In some embodiments, the gene expression response signature was established to distinguish between responsive and non-responsive prior subjects who have received the anti-TNF therapy by a method comprising: mapping genes whose expression levels significantly correlate to clinical responsiveness or non-responsiveness to a human interactome map; and selecting a plurality of genes determined to cluster with one another in a human interactome map, thereby establishing the gene expression response signature.

In some embodiments, the gene expression response signature was established to distinguish between responsive and non-responsive prior subjects who have received the anti-TNF therapy by a method comprising: mapping genes whose expression levels significantly correlate to clinical responsiveness or non-responsiveness to a human interactome map; and selecting a plurality of genes determined to be proximal with one another in a human interactome map, thereby establishing the gene expression response signature.

In some embodiments, the present disclosure provides methods further comprising: validating the gene expression response signature by assigning probability of response to a group of known responders and non-responders; and checking the gene expression response signature against a blinded group of responders and non-responders.

In some embodiments, the responsive and non-responsive prior subjects suffered from the same disease, disorder, or condition.

In some embodiments, the subjects to whom the anti-TNF therapy is administered are suffering from the same disease, disorder or condition as the prior responsive and non-responsive prior subjects.

In some embodiments, the gene expression response signature includes expression levels of a plurality of genes derived from a cluster of genes associated with response to anti-TNF therapy on a human interactome map.

In some embodiments, the gene expression response signature includes expression levels of a plurality of genes proximal to genes associated with response to anti-TNF therapy on a human interactome map.

In some embodiments, the gene expression response signature includes expression levels of a plurality of genes determined to cluster with one another in a human interactome map.

In some embodiments, the gene expression response signature includes expression levels of a plurality of genes that are proximal in a human interactome map.

In some embodiments, genes of the subject are measured by at least one of a microarray, RNA sequencing, real-time quantitative reverse transcription PCR (qRT-PCR), bead array, ELISA, and protein expression.

In some embodiments, a disease, disorder, or condition described herein is an autoimmune disease.

In some embodiments, the subject suffers from a disease, disorder, or condition comprising rheumatoid arthritis, psoriatic arthritis, ankylosing spondylitis, Crohn's disease (adult or pediatric), ulcerative colitis, inflammatory bowel disease, chronic psoriasis, plaque psoriasis, hidradenitis suppurativa, asthma, uveitis, juvenile idiopathic arthritis, vitiligo, Graves' ophthalmopathy (see also thyroid eye disease, or Graves' orbitopathy), multiple sclerosis, or a combination thereof.

In some embodiments, the subject suffers from an autoimmune disease comprising rheumatoid arthritis, psoriatic arthritis, ankylosing spondylitis, Crohn's disease (adult or pediatric), ulcerative colitis, inflammatory bowel disease, chronic psoriasis, plaque psoriasis, hidradenitis suppurativa, asthma, uveitis, juvenile idiopathic arthritis, vitiligo, Graves' ophthalmopathy (see also thyroid eye disease, or Graves' orbitopathy), multiple sclerosis, or a combination thereof.

In some embodiments, the anti-TNF therapy comprises administration of infliximab, adalimumab, etanercept, certolizumab pegol, golimumab, biosimilars, or a combination thereof. In some embodiments, the anti-TNF therapy comprises administration of infliximab or adalimumab.

In some embodiments, the present disclosure provides, in a method of administering anti-TNF therapy, the improvement that comprises administering the therapy selectively to subjects who have been determined to display a gene expression response signature established to distinguish between responsive and non-responsive prior subjects who have received the anti-TNF therapy.

In some embodiments, the responsive and non-responsive prior subjects suffered from the same disease, disorder, or condition.

In some embodiments, the subjects to whom the anti-TNF therapy is administered are suffering from the same disease, disorder or condition as the prior responsive and non-responsive prior subjects.

In some embodiments, the gene expression response signature includes expression levels of a plurality of genes derived from a cluster of genes associated with response to anti-TNF therapy on a human interactome map.

In some embodiments, the anti-TNF therapy comprises administration of infliximab, adalimumab, etanercept, certolizumab pegol, golimumab, biosimilars, or a combination thereof.

In some embodiments, the disease, disorder, or condition is rheumatoid arthritis.

In some embodiments, the disease, disorder, or condition is ulcerative colitis.

In some embodiments, the present disclosure provides use of an anti-TNF therapy in the treatment of a subject determined to display a gene expression response signature established to distinguish between responsive and non-responsive prior subjects who have received the anti-TNF therapy.

In some embodiments, prior to use of the anti-TNF therapy, determining that the subject displays the gene expression response signature. In some embodiments, prior to use of the anti-TNF therapy, determining that the subject does not display the gene expression response signature.

In some embodiments, the gene expression response signature was established to distinguish between responsive and non-responsive prior subjects who have received the anti-TNF therapy by a method comprising: mapping genes whose expression levels significantly correlate to clinical responsiveness or non-responsiveness to a human interactome map; and selecting a plurality of genes determined to cluster with one another in a human interactome map, thereby establishing the gene expression response signature.

In some embodiments, the gene expression response signature was established to distinguish between responsive and non-responsive prior subjects who have received the anti-TNF therapy by a method comprising: mapping genes whose expression levels significantly correlate to clinical responsiveness or non-responsiveness to a human interactome map; and selecting a plurality of genes determined to be proximal with one another in a human interactome map, thereby establishing the gene expression response signature.

In some embodiments, the gene expression response signature was established to distinguish between responsive and non-responsive prior subjects who have received the anti-TNF therapy by the method further comprising: validating the gene expression response signature by assigning probability of response to a group of known responders and non-responders; and checking the gene expression response signature against a blinded group of responders and non-responders.

### Systems and Architecture

In some embodiments, the present disclosure provides a method of validating response to an anti-TNF therapy in a subject, the method comprising: receiving, by a processor of a computing device, a gene expression response signature determined to distinguish between responsive and non-responsive subjects to the anti-TNF therapy; analyzing, by the processor, gene expression of the subject relative to the gene expression response signature to determine whether the subject expresses the gene expression response signature, wherein the gene expression response signature comprising one or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPM1A, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TOR1AIP1, CHN2, RHBDD1, S100A9, KLHL12, GABARAPL1, TMEM97, TLR2, FPR2, ILIB, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGR1CP, FCGR1B, ABL2, GALNT15, FFAR2, ATG4B, FST, NFIL3, RPIA, SMC2, IFIT2, NINJ1, DYRK1A, IRAK2, NUP88, or CHST10.

In some embodiments, the present disclosure provides a system for determining or validating responsiveness to anti-TNF therapy for a subject suffering from a disease, the system comprising: a processor of a computing device; and a memory having instructions stored thereon, wherein the instructions, when executed by the processor cause the processor to perform the methods described herein.

In some embodiments, the present disclosure provides a system for determining or validating responsiveness to anti-TNF therapy for a subject suffering from a disease, the system comprising: a processor of a computing device; and a memory having instructions stored thereon, wherein the instructions, when executed by the processor cause the processor to perform: receiving by the processor, a gene expression response signature determined to distinguish between responsive and non-responsive subjects to the anti-TNF therapy; analyzing, by the processor, gene expression of the subject relative to the gene expression response signature to determine whether the subject expresses the gene expression response signature, wherein the gene expression response signature comprising one or more genes comprising: IGFBP5, IL13RA2, HGF, CXCL2, NR3C1, ABTB1, RGS5, IL7R, TOLLIP, FAM86C1, SOD2, ABHD12, DRAM1, TNC, H2BC3, C16orf58, MEIS1, USP54, INHBA, PPM1A, NAAA, NFE2L1, TRDV2, LIMS1, TAL1, PTK2B, OTX1, ADAMTS12, SNX29, PAPPA, ABCC5, STC1, MS4A7, CXCL6, CEBPB, SLC25A29, CXCL1, CFLAR, PAX5, AP2A2, TRAF1, PLG, PLAU, AMIGO2, EPS15L1, LRRC8C, MAP3K20, SLC35G2, MMP12, LARP4B, AJUBA, SPIRE2, SPPL3, RNF144B, BAD, ZNF57, TRIM8, CANX, NBN, IFIT3, SIAH2, TORIAIP1, CHN2, RHBDD1, S100A9, KLHL12, GABARAPL1, TMEM97, TLR2, FPR2, ILIB, GK3P, SLC7A8, PTGS2, TNFAIP6, SUPV3L1, CSF3R, CLEC4E, TREM1, CREB5, C5AR1, SNCA, BCL6, TARDBP, GK, ECH1, CDCA7L, ECSIT, FPR1, SLC22A4, FCGR3B|FCGR3A, FCGR3B, CXCL8, CXCR1, KIFC1, DUSP1, FCN1, PI15, CXCL11, FGR, MASP1, RIPK2, CCNB1, PLEK, LATS2, CHTOP, TLR4, BAG5, RUVBL2, FGF2, MNDA, IDH3B, BCL2A1, CD82, BMP1, GNAI1, MEFV, SSRP1, AQP9, CYP4F3, TSN, CHEK1, SET, FCGR1CP, FCGR1B, ABL2, GALNT15, FFAR2, ATG4B, FST, NFIL3, RPIA, SMC2, IFIT2, NINJ1, DYRK1A, IRAK2, NUP88, or CHST10.

### Computer Control Systems

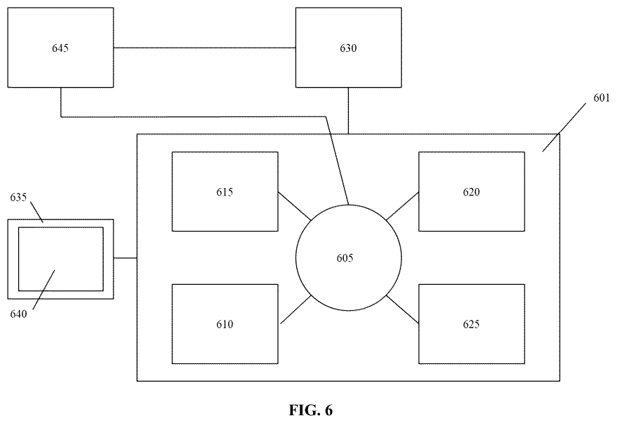

The present disclosure provides computer control systems that can be programmed to implement methods of the present disclosure. FIG. 6 shows a computer system 1101 that is programmed or otherwise configured to generate or develop autoantibody profile or compare autoantibodies with the profile of the specific immune response. The computer system 601 can regulate various aspects of the present disclosure, such as, for example, receive or generate sequence reads, correlate sequences to specific epitopes or autoantibodies, output a result for the user as to the presence of an autoantibody or profile, or an expected progression of a disease. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, selecting autoantibodies for analysis, interacting with graphs correlating autoantibodies to specific generated profiles. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, calculate statistics measurements to identify autoantibodies and generate profiles or predict efficacy and toxicity of a treatment.

It is contemplated that systems, architectures, devices, methods, and processes of the present disclosure encompass variations and adaptations developed using information from the embodiments described herein. Adaptation or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present disclosure that consist essentially of, or consist of, the recited components, and that there are processes and methods according to the present disclosure that consist essentially of, or consist of, the recited processing steps.

It should be understood that the order of steps or order for performing certain action is immaterial so long as the disclosure remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Headers are provided for the convenience of the reader—the presence or placement of a header is not intended to limit the scope of the subject matter described herein.

### EXAMPLES

Examples below demonstrate gene expression response signatures (otherwise referred to as “classifiers” below) characteristic of subjects who do or do not respond to anti-TNF therapy.

### Example 1: Determining Responder and Non-Responder Patient Populations—Ulcerative Colitis

In accordance with the present disclosure, gene expression data from subjects diagnosed with ulcerative colitis (UC) who had received anti-TNF therapy was used to determine patients who are responders and non-responders to anti-TNF therapy. This UC cohort (GSE12251) included 23 patients diagnosed with UC, 11 of which did not respond to anti-TNF-therapy. The gene expression data for this cohort were generated using the Affymetrix® platform.

The gene expression data was analyzed define a set of genes (response signature genes) whose expression patterns distinguish responders and non-responders. To do this, genes with significant gene expression deviations between responders and non-responders were relied on. Unlike other differential expression methods that look for high fold changes in gene expression between two groups, the present disclosure provides the insight that small but significant changes between two groups of patients can be included. The present disclosure thus identifies the source of a problem with other differential expression methods.

Without wishing to be bound by any particular theory, the present disclosure provides an insight that small but significant differences impact responsiveness to therapy. Indeed, the present disclosure notes that, given that patients in these cohorts are all diagnosed with the same disease, they often may not manifest big FCs across genes. The present disclosure demonstrates that even very small but significant changes in gene expression will lead to a different treatment outcome.

Additionally, the present disclosure demonstrates that analysis of genes displaying small (but significant) expression differences, in context of a human “interactome” map, defines signatures that reliably distinguish responders from non-responders.

### In-Cohort Analysis

Using a human interactome (“HI”) map of gene connectivity that reveals features of underlying biology of response and is useful for understanding response signature genes.

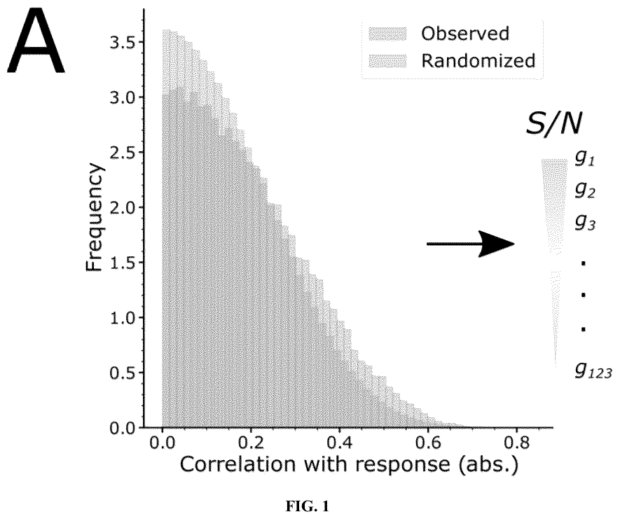

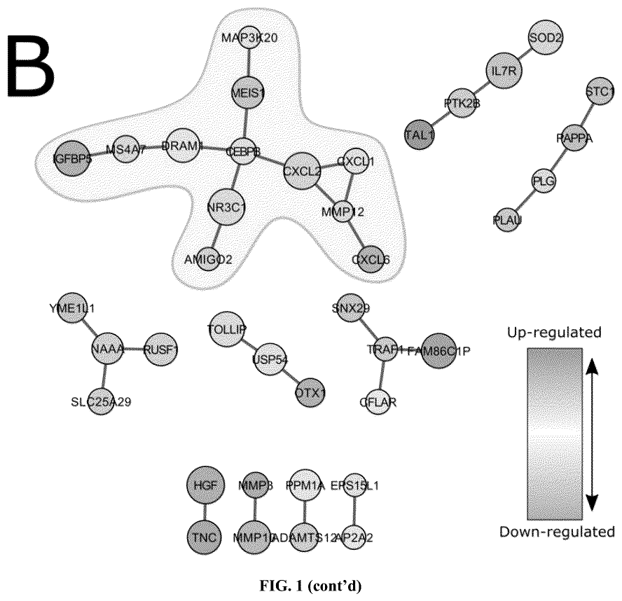

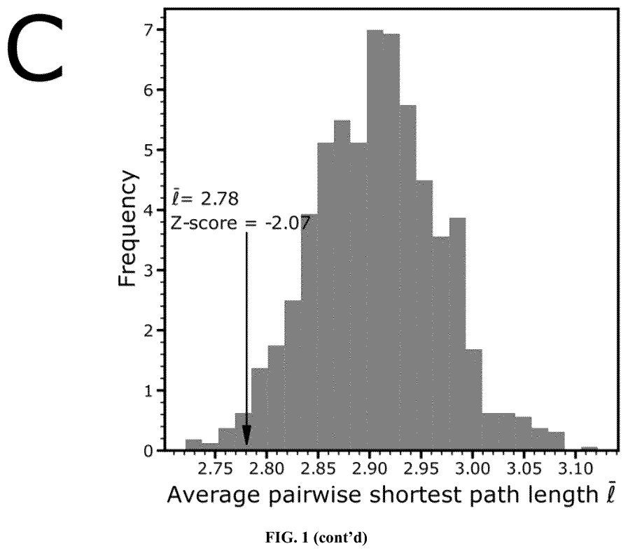

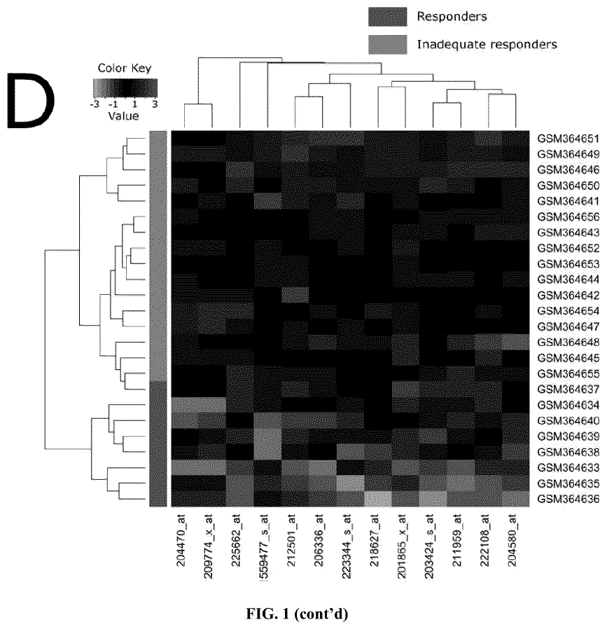

The top 200 genes (as measured by p-value from lowest to highest) whose expression values across patients were significantly correlated to clinical outcome after treatment were selected and mapped to HI. It was observed that even though these genes have been found using the gene expression data only, for example, they form a largest connected component (module) on the HI, and are closer to each other than what is expected by chance HI (FIG. 1, subpanels B-C). Existence of such significant modules (z-score >1.6) has been repeatedly shown to be associated with underlying disease biology. See Barabási, et al. “Network medicine: a network-based approach to human disease,” Nat. Rev. Genet, 12 (1): 56-68 (January 2011); Hall et al,. “Genetics and the placebo effect: the placebome,” Trends Mol. Med., 21 (5): 285-294 (May 2015); del Sol, et al. “Diseases as network perturbations,” Curr. Opin. Biotechnol., 21 (4): 566-571 (August 2010), which are incorporated herein by reference for all purposes.

FIG. 1, subpanel B shows the subnetwork containing the genes correlated to phenotypic outcome in UC cohort as well as their interactions. A number of genes found by gene expression analysis form the LCC of the subgraph. The LCC genes (classifier genes) were then utilized to feed and train a probabilistic neural network.

TABLE 2

| | No. features | | | Shortest path |
|---|---|---|---|---|
| Cohort ID | selected | No. Genes in HI | LCC size | Significance |
| GSE14580 | 123 | 106 | 12 | −2.07 |

Table 2 represents the number and topological properties (e.g., the size of the largest component on the network and its significance) of response signature genes when mapped onto the network.

Compared the method described herein, a major drawback of traditional gene expression analysis is the inability to reproduce the results across different studies. See Ioannidis J. P. A., “Why most published research findings are false,” PLoS Med. 2 (8): e124 (2005); Goodman S. N., et al. “What does research reproducibility mean?” Sci. Transl. Med., 8 (341): 341-353 (2016); Ioannidis J. P., et al. “Replication validity of genetic association studies.” Nat. Genet. 29: (3) 306-309 (November 2001), which are incorporated herein by reference for all purposes. Below, it is shown that the methods and systems described herein are able to make high accuracy predictions across cohorts. To estimate the power of the classifier, the classifier is tested in a completely independent cohort (GSE14580) and in a blinded fashion. The independent UC cohort includes 16 non-responders and 8 responders.

For cross-platform validation, the two cohorts were merged and batch effects removed using the R package, ComBat, a tool used for batch-adjusting gene expression data. See Johnson W. E., et al. “Adjusting batch effects in microarray expression data using empirical Bayes methods,” Biostatistics 8 (1), 118-127 (2007), which is incorporated herein by reference for all purposes. The performance of the designed classifier was tested in the independent cohort (leave-one-batch-out cross validation).

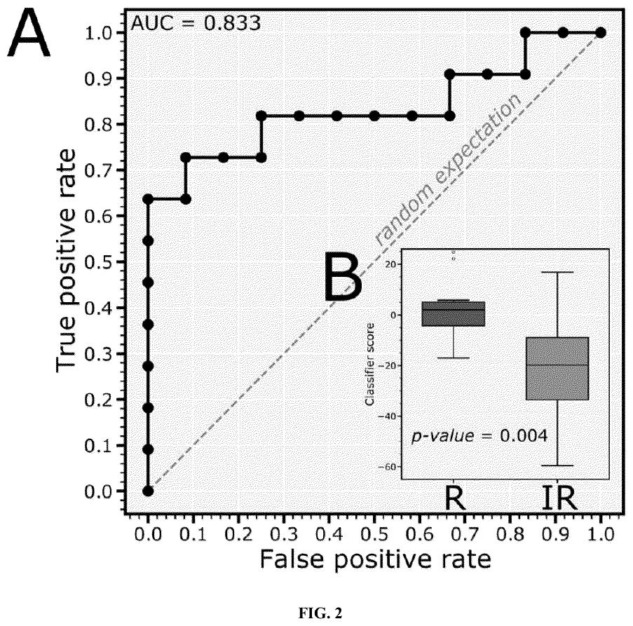

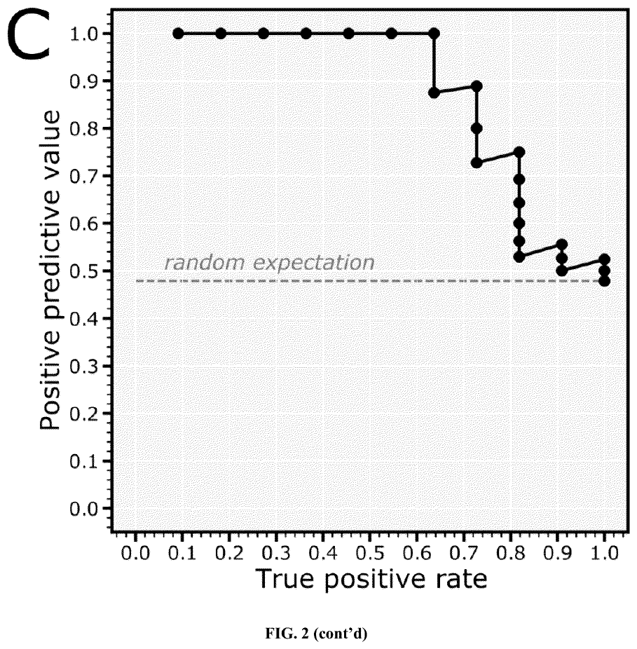

The performance of trained classifiers was validated cross cohort and using an independent data. FIG. 2, subpanels A-B show the receiver operator curves (ROC) as well as positive predictive power (predicting non-responders) of the classifier. The result of the analysis shows an Area Under the Curve (AUC) of 0.83. The classifier is able to detect 64% of the non-responders, with 100%, accuracy, within the validation cohort.

Aside from the high cross-cohort performance assessed by AUC, cross-cohort PPV (Positive Predictive Value) and TPR (True Positive Rate), which indicates the accuracy of detecting non-responders in a blind cohort, were also estimated (FIG. 2, subpanel C). The cross-cohort validation shows that the classifier is able to predict at least 64% of non-responders (PPV=1, TPR=0.64). The classifier is able to detect even more numbers of non-responders (TPR>0.64), which results in slight drop in PPV (FIG. 2, subpanel C). Nevertheless, regardless of the selected point on the curve, the classifier meets or exceeds the commercial criteria (PPV of 0.9 and TPR of 0.5) set by health insurance companies.

### Disease Biology of Non-Responders

The network defined by the analysis described herein provides insights into underlying biology of this response prediction. The classifier genes within the response module were analyzed using GO terms to identify the most highly enriched pathways. We found that inflammatory signaling pathways (including TNF signaling) were highly enriched, as were pathways linked to sumoylation, ubiquitination, proteasome function, proteolytic degradation and antigen presentation in immune cells. Thus, the network approach described herein has captured protein interactions for selecting genes within the response module that clearly reflect the biology of the disease and drug response at the independent patient level and allow the accurate prediction of response to anti-TNF therapies from a baseline sample.

### DISCUSSION

One problem with existing anti-TNF therapy approaches is that “many patients do not respond to the . . . therapy (primary non-response—PNR) or lose response during the treatment (secondary loss of response-LOR).” See, e.g., Roda et al. Clin Gastroentorl. 7:e135, January 2016, which is incorporated herein by reference for all purposes. Specifically, reports indicate that “around 10-30% of patients do not respond to the initial treatment and 23-46% of patients lose response overtime” Id. Thus, overall, the drug response rate for anti-TNF therapy (and in particular for anti-TNF therapy to treat UC patients) is below 65%, resulting in continued disease progression and escalating treatment costs for the majority of the patient population. Moreover, billions of dollars are spent prescribing anti-TNF therapies to patients that don't respond. There is a significant need for development of a technology that can identify responder vs. non-responder subjects, prior to initiation of therapy, at the time that therapy (e.g., a particular dose) is administered, or over time as therapy has been or is received.

Gene expression data has been touted as holding the promise of being able to uncover disease biology of individual patients in complex diseases, but up until now the data has been difficult to interpret, and efforts to develop biomarkers (e.g., expression signatures) for therapeutic responsiveness have failed in cross-cohort validation tests. The present disclosure provides new technologies that, for example, consider relatively small changes in expression levels or participation of genes in relevant parts of the human interactome.

As already noted, the present disclosure demonstrates that projecting baseline gene expression profiles from UC patients that are non-responders to anti-TNF therapy on the HI reveals that such profiles cluster and form a largest connected module that describes the non-responders' disease biology. In accordance with the present disclosure, a classifier developed from genes expressed in this module predicts non-response with a high level of accuracy and has been validated in a completely independent cohort (cross-cohort validation). Furthermore, this classifier meets the commercial criteria set by insurance companies and is therefore ready for clinical development and future commercialization.

### Methods

### Microarray Analysis

Cohort 1, GSE14580: Twenty-four patients with active UC, refractory to corticosteroids or immunosuppression, underwent colonoscopy with biopsies from diseased colon within a week prior to the first intravenous infusion of 5 mg infliximab per kg body weight. Response to infliximab was defined as endoscopic and histologic healing at 4-6 weeks after first infliximab treatment using the MAYO score. Six control patients with normal colonoscopy were included. Total RNA was isolated from colonic mucosal biopsies, labelled and hybridized to Affymetrix R Human Genome U133 Plus 2.0 Arrays.

Cohort 2, GSE12251: Twenty-two patients underwent colonoscopy with biopsy before infliximab treatment. Response to infliximab was defined as endoscopic and histologic healing at week 8 using the MAYO score (P2, 5, 9, 10, 14, 15, 16, 17, 24, 27, 36, and 45 as responders; P3, 12, 13, 19, 28, 29, 32, 33, 34, and 47 as non-responders). Messenger RNA was isolated from pre-infliximab biopsies, labeled and hybridized to Affymetrix® HGU133 Plus_2.0 Array.

### Identification of Classifier Genes

Genes with expression values across patients that were significantly correlated to clinical measures after treatment were selected as best determinants of response. These genes were mapped on the consolidated Human Interactome (“HI”). The consolidated Human Interactome collects physical protein interactions between a cell's molecular components relying on experimental support. The material reported by Barabási et al. in “Uncovering disease-disease relationships through the incomplete interactome,” Science, 347 (6224):1257601 (February 2015), which is incorporated herein by reference for all purposes, provides instruction regarding how to build and curate a Human Interactome. The genes on the Human Interactome are not randomly scattered on the network. Instead, they significantly interact with each other, reflecting the existence of an underlying disease biology module that explains response.

### Human Interactome

As noted, the HI contains experimentally supported physical interactions between cellular components. These interactions were queried from several resources but only selected, for example, those that are supported by experimental validation. Most of the interactions in the HI are from unbiased high-throughput studies such as Y2H. All included data were experimentally supported interactions that have been reported in at least two publications. These interactions include, regulatory, metabolic, signaling and binary interactions. The HI contains about 17 k cellular components and over 200K interactions among them. Unlike other interaction databases, no computationally inferred interaction were included, nor any interaction curated from text parsing of literature with no experimental validation.

### Classifier Design and Validation

Genes identified above were used as features of a probabilistic neural network. The classifier was validated using leave-one-out or k-fold cross validation within a given cohort. The classifier was trained based on the outcome data provided on all patients but the one left out. The classifier was blind to the response outcome of that left out patient. Predicting the outcome of the patient that has been left out then validated the trained classifier. This procedure was repeated so that each patient was left out once. The classifier provided a probability for each patient reflecting whether they belong to responder or non-responder group. The logarithm of likelihood ratio was used to assign a score to each patient. Patients were then ranked based on their score and prediction accuracy values were estimated by varying the classifier threshold resulting in the ROC curves. In particular, each patient is given a score by the trained classifier. The prediction accuracy is measured for the entire cohort as a whole and by checking whether given scores across patients well distinguish responders and non-responders. Prediction performance is generally measured by the Area Under the Curve (AUC). When higher levels of accuracy are required, positive predictive value (PPV) and true positive rate (TPR) can be used. The score cutoff that results in best group separation (e.g., highest PPV) is set for future predictions.

### Example 2: Determining Responder and Non-Responder Patient Populations—Rheumatoid Arthritis

Analogous to Example 1, the present Example 2 describes prediction of response or non-response to anti-TNF therapy in patients suffering from rheumatoid arthritis (RA). The presently described predictions satisfy the performance threshold identified by payers and physicians of Negative Predictive Value (NPV) of 0.9 and True Negative Rate (TNR) of 0.5. As described elsewhere, positive predictive value (PPV) and true positive rate (TPR) can also be associated with or determined from combinations of true negative rate (TNR), negative predictive value (NPV), false positive rate, false negative rate, sensitivity, and specificity.

In the present example, gene expression data from baseline blood samples for two cohorts comprising a total of 89 RA patients were analyzed. The methodology utilized in the present Example to develop a classifier (e.g., a gene expression response signature) that predicted response or non-response to anti-TNF therapy included a process wherein initial genes were selected based on differential expression between responders and non-responders to anti-TNF therapy; such genes were projected on the human interactome to determine which genes form a significant and biologically relevant cluster; genes that cluster on the interactome were selected and fed into a probabilistic neural network (PNN) to develop the final classifiers; and each classifier was validated using leave-one-out validation in the training set and validated cross-cohort in an independent cohort of patients (test set). For RA, the final classifier contained 9 genes and reached an NPV of 0.91 and TNR of 0.67 in the test set.

The developed classifiers meet the performance thresholds set by payers and physicians; those practicing the present disclosure will appreciate that these classifiers are useful tests that predict non-response to anti-TNFs prior to initiation of therapy or to assess desirability of altering administered therapy. Among other things, provided technology therefore permits selection of therapy (whether initial therapy or continued or altered therapy), including enabling patients to be switched onto alternative therapies faster, resulting in substantial clinical benefits to patients and savings to the healthcare system.

### Data Description

The response prediction analysis in RA utilized in the present Example was based on two individual cohorts (Table 3 and Table 4). Response was measured 14-weeks after initiation of anti-TNF therapy, with response rates (Good responders; DAS28 improvement>1.2, corresponding to LDA or remission) in cohort 1 and 2 of 30% and 23%, respectively. Cohort 1 was used to train the classifier and cohort 2 was used as the independent test cohort to validate the predictive power of the classifier.

The analyses were conducted on RNA expression data generated from whole blood, before initiation of therapy, using an Illumina® BeadArray platform and provided as standard output of BeadStudio. Raw data was normalized and processed using lumi package in R.

TABLE 3

| Clinical response according to | | |
|---|---|---|
| EULAR DAS28 criteria | Cohort 1 | Cohort 2 |
| No. Good responders | 15 | 9 |
| No. Moderate responders | 15 | 15 |
| No. Non-responders | 20 | 15 |

TABLE 4

DAS28 Improvement

| Baseline DAS28 | >1.2 | >0.6 & ≤1.2 | ≤0.6 |
|---|---|---|---|
| ≤3.2 | Good | Moderate | No response |
| | response | response | |
| >3.2 & ≤5.1 | Moderate | Moderate | No response |
| | response | response | |
| >5.1 | Moderate | No response | No response |
| | response | | |

### Identifying Classifier Genes

Expression values for over 10,000 probes (genes) were available in each patient; those practicing the present disclosure will appreciate the challenges associated with defining a set of genes (features) that effectively distinguishes response from such a volume of data. Insights provided by the present disclosure, including that particularly useful genes for inclusion in a classifier may, in some embodiments, be those with relatively small changes, permit effective selection of gene (feature) set(s) for use in a classifier.

In the present Example, genes for inclusion in an RA classifier were selected via an analysis process comprising: genes were ranked based on their significance of correlation to patient's response outcome (change in baseline DAS28 score at week 14) using Pearson correlation resulting in 200 top ranked genes (Feature set 1). Unlike other differential expression methods that look for highest fold changes in gene expression between two groups, the present Example captures small but significant changes between two groups of patients.

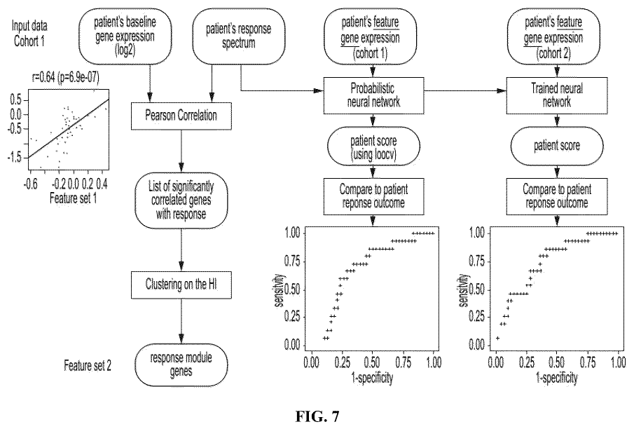

Second, the present disclosure appreciates that gene products (proteins) do not function in isolation, and furthermore appreciates that reference to the interactome-a map of protein interconnectivity—can valuably be used as a blueprint to understand roles played by individual gene products in context (e.g., in biology of cells or organisms). By mapping the 200 genes identified above on the interactome, a significant cluster, or response module, consisting of 41 proteins was identified (Table 5). Existence of a significant cluster was repeatedly shown to be associated with underlying disease biology. The observed response module not only uncovers the underlying biology of response but also served as Features set 2. As an example, FIG. 7 illustrates a classifier development flowchart containing identifying features of the classifier (A), training and validation of a probabilistic neural network on cohort 1 using identified features (B) and validation of the trained classifier using identified feature genes expressions in an independent cohort (C). The final set of features are selected based on best performance.

TABLE 5

| | # Top genes | | | |
|---|---|---|---|---|
| Cohort ID | selected | # Genes in HI | LCC size | Significance |
| 1 | 200 | 186 | 41 | 1.19 |

### Training the Response Classifier and In-Cohort Validation

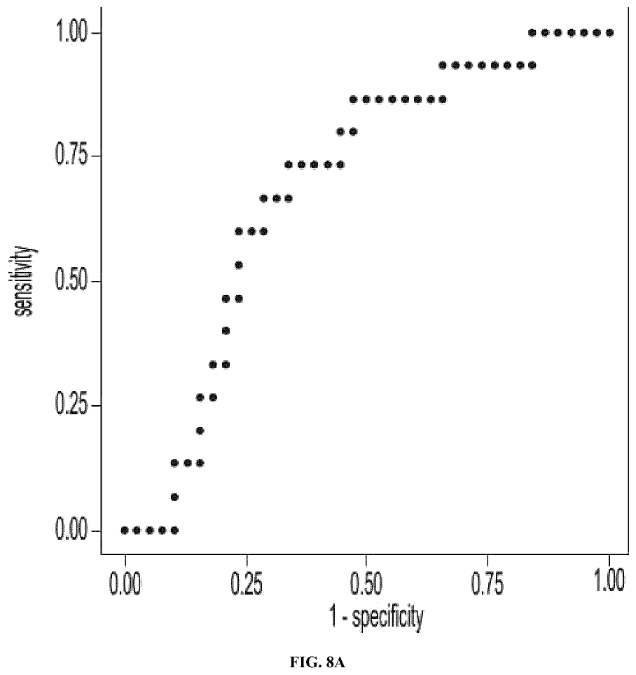

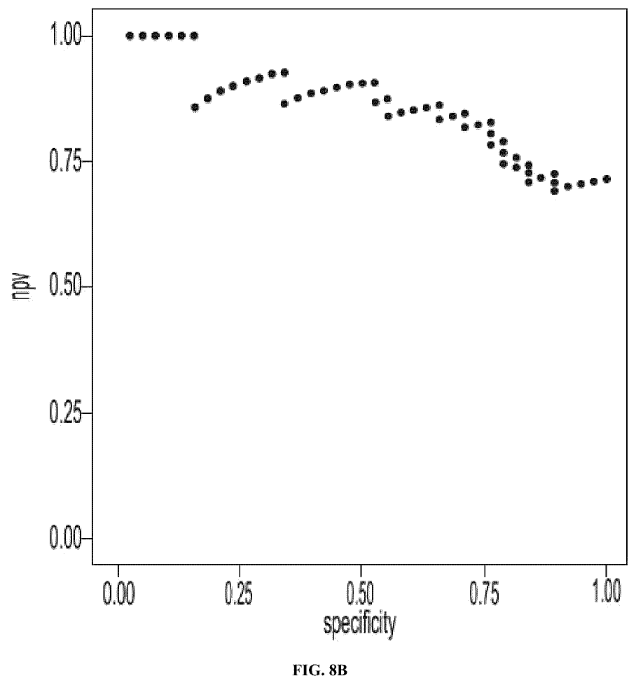

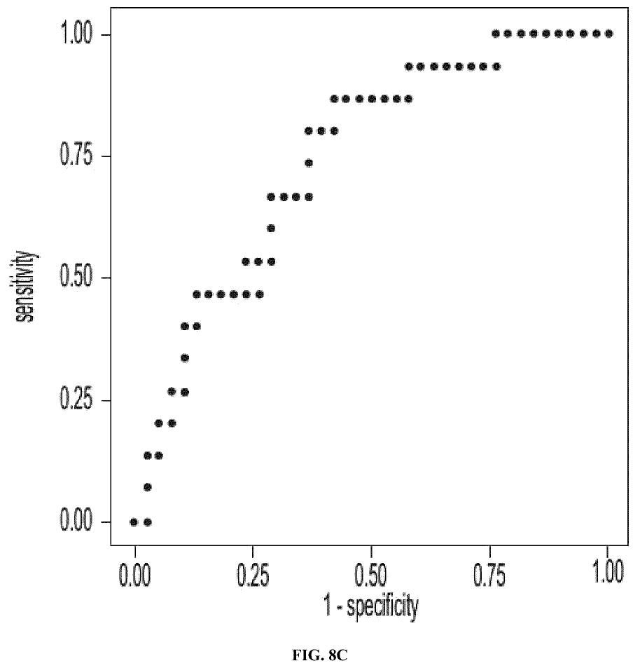

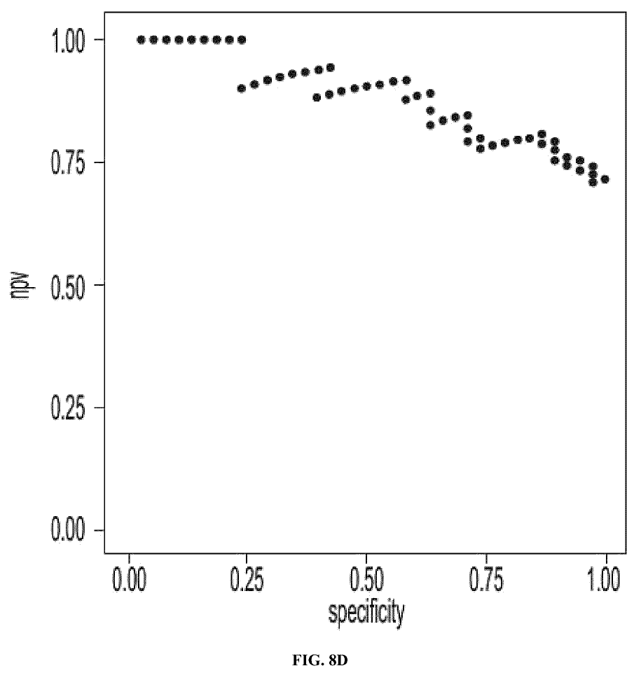

In the present Example, a response classifier was trained by feeding a probabilistic neural network with Feature set 1 and 2. Training the classifier on Feature set 1 significantly predicted response using leave-one-out cross validation and reached an AUC of 0.69, an NPV of 0.9 and a TNR of 0.52 (FIG. 8A, and FIG. 8B, respectively), outperforming Feature set 2. Having a smaller number of classifier genes also opens up the opportunity to use a variety of lower cost, FDA-approved expression platforms with a broad installed base to generate the required gene expression data sets. The classifier was therefore further trained to see if performance holds up when reducing the number of genes in Feature set 1 by training on top n-ranked genes where n goes from 1 to 20. A local maximum was observed in classifier performance when training on the top 9 genes (AUC=0.74, corrected p-value=0.006) with an NPV of 0.92 and a TNR of 0.76 (FIG. 8C and FIG. 8D). The 9-gene classifier was chosen for the cross cohort validation analysis below.

### Validation of Trained Response Classifier in an Independent Cohort (Cross-Cohort Validation)

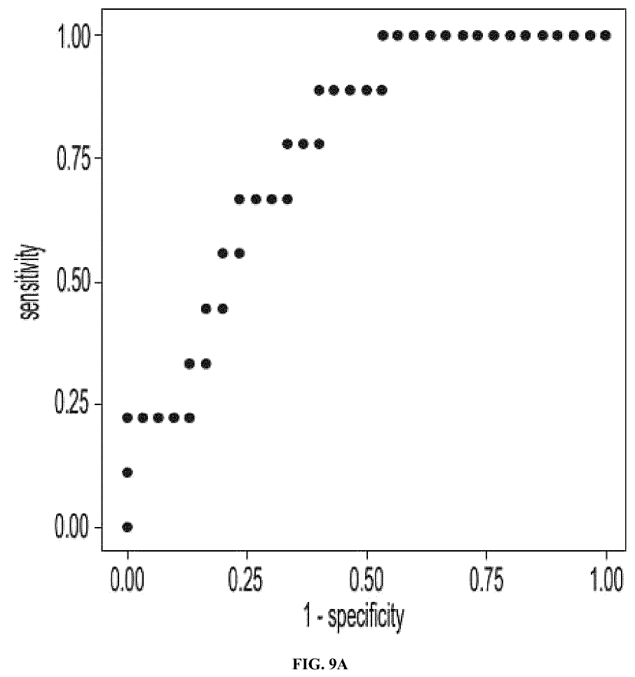

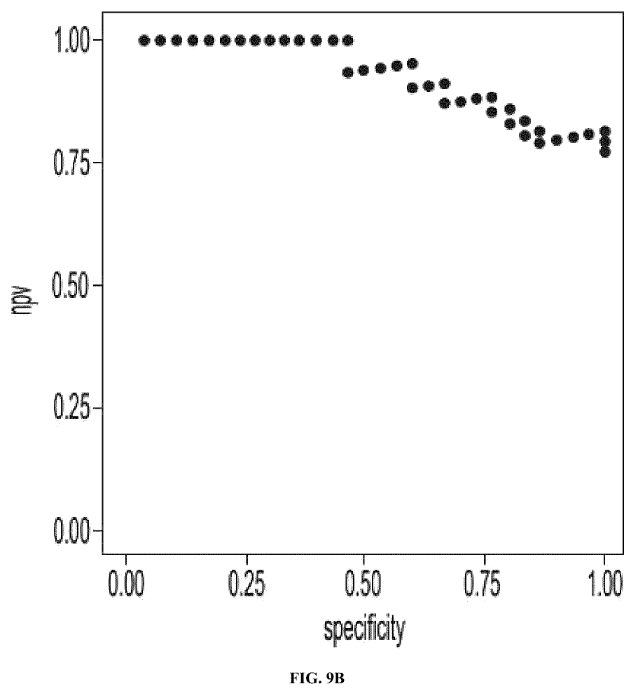

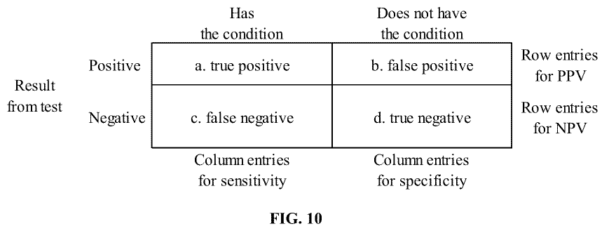

Of importance when building diagnostic tests and classifiers is the ability to reproduce the results and successfully test the classifier's performance in an independent cohort. The developed 9-gene classifier was therefore tested in a blinded fashion on a completely independent group of patients (cohort 2). The results show that the classifier performed well (cross-cohort AUC=0.78, p value=0.01) with an NPV of 0.91 and a TNR of 0.67 (FIG. 9B and Table 6). FIG. 9A is an ROC curve of cross-cohort classifier test results.

TABLE 6

| Predicting non-responders for TNF-naïve patients | AUC | NPV | TNR |
|---|---|---|---|
| Classifier trained on cohort 1 tested on cohort 2 | 0.78 | 0.91 | 0.67 |

### DISCUSSION

The present Example documents effectiveness of a classifier, as described herein, that predicts non-response to anti-TNF drugs before therapy is prescribed in patients suffering from RA.

Interviews with payers and clinicians indicate that current target specifications aim to identify at least half of the non-responders to anti-TNF therapy with high negative predictive accuracy (NPV>90%). Patients that are identified as non-responders can be placed on alternative effective therapies and higher response rates for those patients still offered anti-TNFs can be achieved. Financial savings are garnered by not spending on expensive ineffectual therapies and avoiding serious side effects and continuing disease progression. By identifying 50% of the non-responders, significant cost and care benefits can be achieved since, in the absence of stratification, two-thirds of patients do not achieve the target of LDA or remission today. High NPV is desired to ensure that few patients that may have responded are not incorrectly withheld a therapy they may have benefited from.

For RA, the present disclosure has demonstrated an AUC of 0.78, an NPV of 0.91 and a TNR of 0.67, resulting in the matrix below (Table 7). That is, the classifier identifies 67% of true non-responders with a 91% accuracy. Stratifying patients using this classifier can increase the response rate for the anti-TNF treated group by 71% from 34% to 58%. By comparison, the highest cross-cohort performance reported for classifiers developed by others had an NPV of 0.71 and a TNR of 0.71. See Toonen EJ. et al. “Validation study of existing gene expression signatures for anti-TNF treatment in patients with rheumatoid arthritis.” PLoS One. 2012; 7 (3):e33 199, which is incorporated herein by reference for all purposes. Using that classifier may significantly misclassify the genuine responders leading to a worse overall response rate than not using it at all. The presently described classifiers clearly meet the performance targets when tested in an independent cohort of patients.

TABLE 7

Predicted

| | | R | NR | | |
|---|---|---|---|---|---|
| Actual | R | 30 | 4 | 34 | TPR 87% |
| | NR | 22 | 44 | 66 | TNR 67% |
| | | 52 | 48 | | |
| | | PPV 58% | NPV 91% | | |

The reduced number of genes in the classifier allows several expression analysis platforms to be considered for the delivery of the final commercial version of the test. For example, Nanostring nCounter system uses digital barcode technology to count nucleic acid analytes for panels of up to several hundred genes on an FDA approved platform. Multiplexed qRT-PCR is the gold standard for quantifying gene expression for panels of less than ˜20 genes and can enable the test to be offered as a distributable kit. RA is a chronic, complex auto-immune diseases, where many genetic risk factors have been identified but none of them are of sufficient impact to be useful as diagnostic or prognostic markers. The present disclosure provides a ranked list of candidate genes based on correlation of baseline expression level with response outcomes. The rank order is derived from the significance of the correlation. The present disclosure, however, does not prioritize genes with larger fold change across the category of responders and non-responders. It is common practice in the field to give preference to genes that show the highest fold change. This is because it is generally believed that large changes in expression levels are biologically more meaningful, and because of the technical advantage of high signal to noise ratios to compensate for high background and other sources of technical variability. However, the present disclosure appreciates that small differences, which are ignored or overlooked in other technologies, can provide important, and even critical, discriminating capability. Without wishing to be bound by any particular theory, the present disclosure proposes that subtle differential perturbations may be particularly relevant or important in situations, like the present, where subjects suffering from the same disease, disorder, or condition are compared with one another (e.g., rather than with “control” subjects not suffering from the disease, disorder, or condition). It may be that small yet statistically significant differences in gene expression differentiate patient populations in complex diseases such as RA. This study shows that even very small but significant changes in gene expression will lead to a different treatment outcome. This method captures genes that are overlooked by other differential expression methods.

Additionally, the present disclosure utilizes the highly unbiased and independently validated map of the protein-protein interactions in cells, the human interactome. By mapping the prioritized genes to the interactome, distinct and statistically significant clusters appear. In addition to using the interactome network analysis to define the classifier, the identified clusters also provide biological insights into the biology and causal genes of anti-TNF response. The genes corresponding to the top 9 genes in RA are valuable in immunological pathways and functions linked to ER stress, the protein quality control pathway, control of the cell cycle and the ubiquitin proteasome system, primarily in targeting key regulators of the cell cycle to the proteasome through ubiquitination.

The classifiers described here serve as the basis for diagnostic tests to predict anti-TNF non-response for patients with moderate to severe disease and considering initiating biologic therapy. Patients identified as non-responders will be offered alternative, approved mechanism of action therapies. These tests will provide significant improvements to current clinical practice by increasing the proportion of patients reaching treatment goals, making the treatment assignment based on scientific data and as a result decrease waste of resources and generate significant financial savings within the health care system.

### Materials and Methods

### RA Cohort Description and Microarray Analysis

Blood samples were collected from RA patients across the United States from two individual observational studies, both of which predominantly consisted of older Caucasian women. Cohort 1 was obtained from a multi-center study conducted in 2014. These patients were treated with Enbrel®, Remicade®, Humira®, Cimzia® and Simponi®. Cohort 2 was obtained from the Autoimmune Biomarkers Collaborative Network, a NIAMS supported contract to develop new approaches to biomarkers for RA and lupus in 2003. These patients were treated with Humira®, Remicade® and Enbrel®.

The level of response was defined using the EULAR DAS28 scoring criteria assessed 14 weeks after anti-TNF treatment. EULAR response rates for female TNF naïve patients are given in Table 3. EULAR response characterizes patients into good responders, moderate responders and non-responders. For this study, response was defined as EULAR good response, or DAS28 improvement>1.2. This corresponds to LDA or remission.

The gene expression data and 14 week response outcome was available for 50 and 39 female and TNF naïve samples in cohort 1 and 2, respectively, for classifier design and validation.

All subjects had Pax Gene tubes drawn at baseline before starting therapy, and again at 14 weeks after treatment started. RNA was isolated using the QIAcube (Qiagen) following the manufacturer's automated protocol for PaxGene blood RNA. Extracted samples were eluted in 80 ul of elution buffer (BR5) and subsequently run on Agilent's 2100 Bioanalyzer of RNA integrity using the RNA 6000 Nanochip. Samples with RNA Integrity Numbers (RIN)>6.5 were diluted to 30 ng/μl in a total 11 μl of RNAse-free water. Samples were amplified using Life Technologies Illumina® RNA Total Prep Amplification Kit. 750 ng of cRNA was re-suspended in 5 μl of RNAse-free water for analysis on the Illumina® Human HT-1.2v4 chip (cohort 1 samples) and 1.2 μg was re-suspended in 10 μl of RNAse-free water for analysis Illumina® WG6v3 Bead Chip (cohort 2 samples). All samples were processed according to the manufacturer's instructions.

Raw data were exported from GenomeStudio® and further analyzed with the R programming language. All datasets were background corrected using the R/Bioconductor package “lumi.” Data were further transformed using variance stabilization transformational (vst) and quantile normalized. Probes with zero detection count and detection rates of lower that 50% across samples were removed from the study. To enable cross cohort classifier testing, the two cohorts were combined and normalized using the ComBat package in R and then separated to ensure completely blind testing. All of the microarray analysis resulted in having about 10,000 common probes in the two cohorts.

### Identification of Classifier Genes

Genes with expression values that are significantly correlated to clinical measures after treatment are selected as the best determinants of response. Expression correlation of gene expression to response outcome is measured by Pearson correlation. Genes are ranked based on the correlation value and the performance of the classifier is assessed when using top n ranked genes. In some cases mapping the ranked genes on the interactome forms a significant cluster reflecting the under lying biology of response. Itis observed that the ranked genes are not randomly scattered on the network. Instead, they significantly interact with each other, reflecting the existence of an underlying disease biology module that explains response.

### Classifier Design and Validation

Genes identified previously were used as features of a probabilistic neural network. In this approach the average distance of each sample to training samples' probability distribution functions is calculated. The average distance of a test sample to training samples in the n-dimensional feature space determines the probability of belonging to one group vs. the other. The classifier was validated using leave-one-out cross validation within a given cohort. In this approach, the classifier was trained based on the outcome data provided on all patients but the one left out. The classifier was blind to the response outcome of that left out patient. Predicting the outcome of the patient that has been left out then validated the trained classifier. This procedure was repeated so that each patient was left out once. The classifier provided a probability for each patient reflecting whether they responded or not. These probabilities were used to define a score (by using log of likelihood ratio) for each patient. The area under the curve (AUC) determined the performance of the classifier. In cross-cohort assessment of the classifier, the trained classifier was completely blind to the outcome of the independent cohort. Trained data on one cohort is tested to determine its ability to predict response in an independent cohort.

### Statistical Analysis

Fisher's t-test was used to determine the significance of difference between two distributions.

### Human Interactome

The human interactome contains experimentally supported physical interactions between cellular components. These interactions are collected from several resources and those supported by a rigorous experimental validation confirming the existence of a physical interaction between proteins are selected. Most of the interactions in the interactome are from unbiased high-throughput studies such as yeast 2-hybrid. Experimentally supported interactions that that have been reported in at least two publications are also included. These interactions include regulatory, metabolic, signaling and binary interactions. The interactome contains about 17,000 cellular components and over 200,000 interactions. Unlike other interaction databases the present methods do not include any computationally inferred interactions, nor any interaction curated from text parsing of literature with no experimental validation. Therefore, the interactome used is the most complete, carefully selected and quality controlled version to date.

### Example 3: Determining a Gene Expression Response Signature—Ulcerative Colitis

The present examples provide a network-based response module comprised of gene expression biomarkers that predict response or non-response to an anti-TNF therapy (also referred to as TNF inhibitors, or, “TNFi” or “TNFis”, including infliximab) at treatment initiation in ulcerative colitis.

### Cohort Description

In the present example, two cohorts were studied. Cohort A (GSE14580) included twenty-four patients with active ulcerative colitis (UC), refractory to corticosteroids or immunosuppression, and underwent colonoscopy with biopsies from diseased colon within a week prior to the first intravenous infusion of 5 mg infliximab per kg body weight. Response to infliximab was defined as endoscopic and histologic healing at 4-6 weeks after first infliximab treatment. Eight patients were determined to be responders, sixteen were determined to be non-responsive. Six control patients with normal colonoscopy were included. Total RNA was isolated from colonic mucosal biopsies, labelled, and hybridized to Affymetrix® Human Genome U133 Plus 2.0 Arrays.

Cohort B (GSE12251) included twenty-two patients who underwent colonoscopy with biopsy before infliximab treatment. Response to infliximab was defined as endoscopic and histologic healing at week 8 (12 patients as responders and 11 patients as non-responders). Messenger RNA was isolated from pre-infliximab biopsies, labeled and hybridized to Affymetrix Human Genome U133 Plus_2.0 Array.

### Microarray Analysis

The two datasets were downloaded using GEOquery R package. Before treatment gene expression data were extracted by setting the visit time point to baseline. Probe IDs were converted to gene Entrez ID using the hgu133plus2.db database. The two datasets were merged by the common probe IDs. Batch effects were removed using ComBat from the sva R package. To retain the biological differences between responders and non-responders, cohort-specific biomarkers were derived prior to applying ComBat.

### Human Interactome

The Human Interactome, previously described in Menche et al. Science, 347 (6224): 1257601 (Feb. 20, 2015), which is incorporated herein by reference for all purposes, contains experimentally determined physical interactions between proteins. These interactions include, regulatory, metabolic, signaling, and binary interactions. The Human Interactome amalgamates data from more than 300 thousand interactions among them.

### Identification of Classifier Genes (e.g., Genes of the Gene Expression Response Signature)

For all genes in each cohort, Pearson correlation between their gene expression values and response to treatment was determined. The signal-to-noise ratio of each gene correlation was calculated by randomly shuffling of the response outcome 100 times. Selected genes were then mapped onto the consolidated Human Interactome, and the largest connected component (LCC), was determined.

### Classifier Design and Validation

Genes identified as discriminatory between responders and non-responders to infliximab that were in the LCC were used as features of a probabilistic neural network. González-Camacho, et al. BMC Genomics. 17:208. (Mar. 9, 2016), which is incorporated herein by reference for all purposes. One cohort was selected for classifier training using the R package pnn, while the second cohort was used for blinded independent validation. The in-cohort model training and validation was done using a leave-one-sample-out cross validation where the classifier was blind to the response outcome of that left-out patient. The classifiers were validated using leave-one-batch out cross-validation where one cohort was used for feature selection and model training and the other cohort was used for independent validation.

The classifier was trained using the default smoothing parameter (σ=0.8).

The classifier provided a probability for each patient reflecting whether or not that individual responded to infliximab. The log likelihood ratio of response and non-response probabilities were used to define a score for each patient and draw the receiver operating characteristic (ROC) curves by comparing the score to actual response outcomes. The area under the curve (AUC) determined the performance of the classifiers. In cross-cohort assessment of classifiers, the trained classifiers were blind to the outcome of the independent cohort.

### Results

Identification of gene expression features predictive of non-response to infliximab

To identify genes whose expression best distinguishes responders from non-responders (also referred to as “inadequate responders”) to infliximab, two publicly available UC patient gene expression datasets were downloaded for which the clinical outcomes data were available. Arijs I, et al. Gut. 58 (12): 1612-9 (2009), which is incorporated herein by reference for all purposes. Each cohort was separately analyzed to find genes with significant gene expression deviations between responders and inadequate responders. Santolini M, et al. NPJ Syst Biol Appl. 4:12 (2018), which is incorporated herein by reference for all purposes. Unlike other differential expression methods that look for large fold-changes in gene expression between two groups, this analysis investigated small but significant changes—a high signal-to-noise ratio—between the two cohorts. Genes were ranked by decreasing value of signal-to-noise ratio and the top 123 probes with the highest signal-to-noise ratio were selected as infliximab response discriminatory genes (FIG. 5).

### Refinement of Molecular Signature Genes Using the Human Interactome

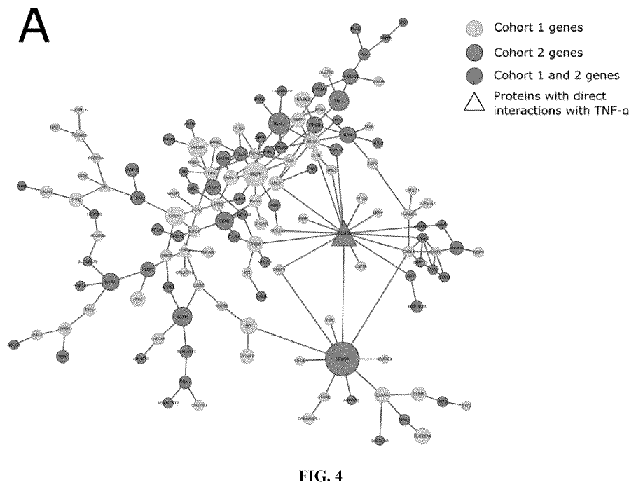

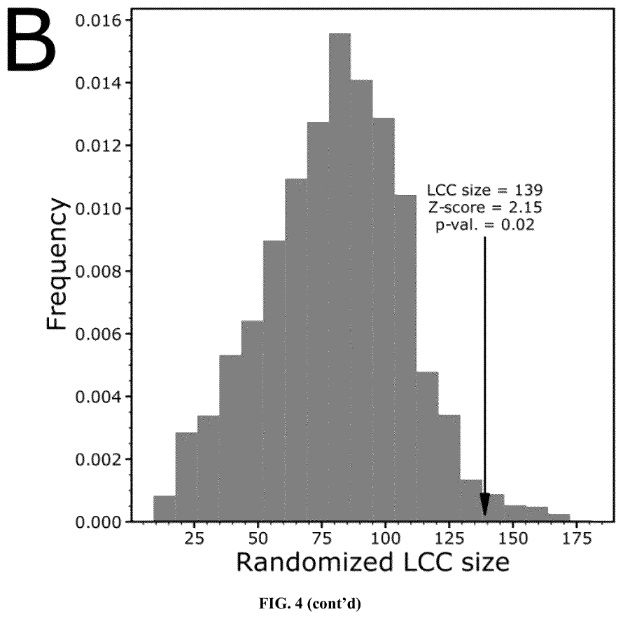

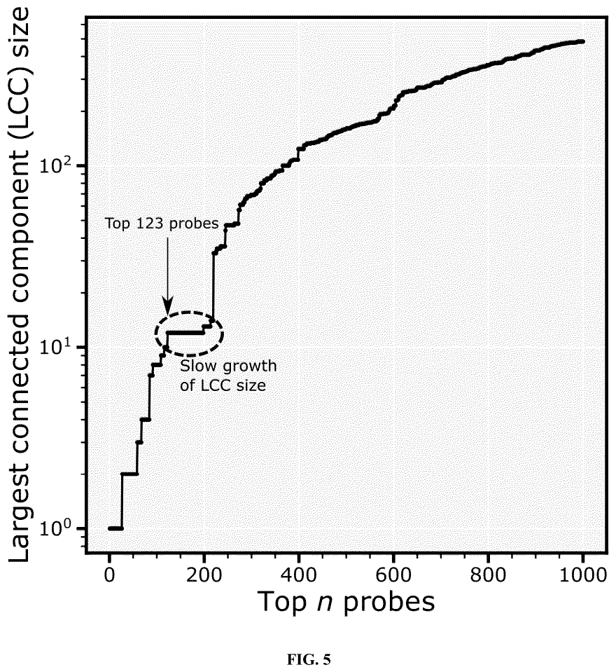

The Human Interactome network map of protein-protein interactions can serve as a blueprint to better understand the interconnectivity and underlying biology of the response prediction genes. The top 200 probes from each cohort whose expression values across patients were significantly correlated to clinical outcome after infliximab treatment were selected and their associated gene IDs were mapped onto the Human Interactome (FIG. 4, subpanels A-B). Although these genes were identified from gene expression data only, for example, the proteins encoded by these genes formed a significant cluster on the Human Interactome. The formed LCC contains 139 genes, four of which belong to both cohorts. The LCC on the Human Interactome was larger than expected by chance (z-score of 2.15). Menche J, etal. Science. 347 (6224): 1257601 (2015); Sharma A, et al. Hum Mol Genet. 24 (11): 3005-20 (2015); Barabasi A L, et al., Nat Rev Genet. 12 (1): 56-68 (2011); Ghiassian S D, et al. Sci Rep.6:27414 (2016), which are incorporated herein by reference for all purposes. Z-scores >1.6 have been associated with sub-networks of underlying disease biology. Amongthe lists of LCC genes, four genes (HGF, SOD2, PAPPA, STC1) were in common between the two cohorts. See Table 8, below for list of genes within the LCC.

TABLE 8

| | |
|---|---|
| Cohort A | Cohort B |

| | | | |
|---|---|---|---|
| ABCC5 | MS4A7 | ABL2 | GNAI1 |
| ABHD12 | NAAA | AQP9 | HGF |
| ABTB1 | NBN | ATG4B | IDH3B |
| ADAMTS12 | NFE2L1 | BAG5 | IFIT2 |
| AJUBA | NR3C1 | BCL2A1 | IL1B |
| AMIGO2 | OTX1 | BCL6 | IRAK2 |
| AP2A2 | PAPPA | BMP1 | KIFC1 |
| BAD | PAX5 | C5AR1 | LATS2 |
| C16orf58 | PLAU | CCNB1 | MASP1 |
| CANX | PLG | CD82 | MEFV |
| CEBPB | PPM1A | CDCA7L | MNDA |
| CFLAR | PTK2B | CHEK1 | NFIL3 |
| CHN2 | RGS5 | CHST10 | NINJ1 |
| CXCL1 | RHBDD1 | CHTOP | NUP88 |
| CXCL2 | RNF144B | CLEC4E | PAPPA |
| CXCL6 | S100A9 | CREB5 | PI15 |
| DRAM1 | SIAH2 | CSF3R | PLEK |
| EPS15L1 | SLC25A29 | CXCL11 | PTGS2 |
| FAM86C1 | SLC35G2 | CXCL8 | RIPK2 |
| H2BC3 | SNX29 | CXCR1 | RPIA |
| HGF | SOD2 | CYP4F3 | RUVBL2 |
| IFIT3 | SPIRE2 | DUSP1 | SET |
| IGFBP5 | SPPL3 | DYRK1A | SLC22A4 |
| IL13RA2 | STC1 | ECH1 | SLC7A8 |
| IL7R | TAL1 | ECSIT | SMC2 |
| INHBA | TNC | FCGR1B | SNCA |
| KLHL12 | TOLLIP | FCGR1CP | SOD2 |
| LARP4B | TOR1AIP1 | FCGR3B | SSRP1 |
| LIMS1 | TRAF1 | FCGR3B\|FCGR3A | STC1 |
| LRRC8C | TRDV2 | FCN1 | SUPV3L1 |
| MAP3K20 | TRIM8 | FFAR2 | TARDBP |
| MEIS1 | USP54 | FGF2 | TLR2 |
| MMP12 | ZNF57 | FGR | TLR4 |
| | | FPR1 | TMEM97 |
| | | FPR2 | TNFAIP6 |
| | | FST | TREM1 |
| | | GABARAPL1 | TSN |
| | | GALNT15 | |
| | | GK | |
| | | GK3P | |

### Classifier Training and Blinded Cross-Cohort Validation

For each cohort, the LCC genes were used to train a probabilistic neural network. See Specht DF. IEEE Transactions on Electronic Computers. EC-16 (3): 308-19 (1967); Specht DF. IEEE Trans Neural Netw. 1 (1): 111-21 (1990). A probabilistic neural network is an optimum pattern classifier that minimizes the risk of incorrectly classifying an object with high efficiency. Gonzalez-Camacho J M, et al. BMC Genomics. 17:208 (2016), which is incorporated herein by reference for all purposes. For each cohort, the probabilistic neural networks were trained using the LCC genes and patient data to teach the predictive classifiers the appropriate patient outcome (e.g., response or inadequate response to infliximab) for each input (e.g., gene expression levels of LCC genes).

Blinded, independent cross-cohort validation assessed the performance of the two predictive classifiers. In this analysis, the classifier that was trained on the known data and outcomes from one cohort was used to predict the outcomes on the other cohort, ultimately testing the ability of the predictive classifiers to accurately predict inadequate response to infliximab in an unseen patient population. To assess the performance of the classifiers, the classifier predicted probabilities were converted to a continuous classifier prediction score using log-likelihood ratio. ROC curves, which plot the rate of false positives versus the rate of true positives, were used to assess cross-cohort performance (FIG. 2, subpanel A). An AUC of 0.83 was observed for classifier trained on cohort A predicting response to infliximab among cohort B patients. At about 90% positive predictive value (PPV), the classifier had a sensitivity of at least 70%. The distribution of classifier prediction scores in responders and inadequate responders when validated in independent cohorts showed a significant difference between the classifier prediction scores for responders and inadequate responders (FIG. 2).

### The UC Infliximab Response Module is a Sub-Network on the Human Interactome

The high cross-cohort performance, despite the limited overlap between LCC gene sets, motivated the search for an underlying mechanism that explained the biology of inadequate response to infliximab in UC patients. When the 200 top probes from the two cohorts were mapped simultaneously onto Human Interactome, the genes were not randomly scattered on the network, but instead significantly interacted with each other (z-score of 2.15) forming a common LCC (FIG. 4) that was significantly larger than the random expectation (139 genes; z-score of 2.15). To account for genes that were shared between the two cohort gene lists,, a careful randomization was made to estimate the significance of interconnectivity. Three proteins in the common LCC (FFAR2, GK and CEBPB) are direct interaction partners of TNF-α, the protein target of infliximab. Several proteins in the common LCC were orphan genes that were not previously part of LCCs of the individual cohorts (e.g., IGFBP5 and IL13RA2) and yet were integrated into this common LCC (FIG. 4, subpanel A). Our results show that even though the biomarkers identified from each cohort were apparently distinct with minimal overlap, their protein products tend to interact significantly on the network, reflecting the existence of an underlying disease biology sub-network, or response module, that defines a molecular signature of inadequate response to infliximab in UC patients.

### DISCUSSION

This present example describes two predictive classifiers developed using knowledge from the Human Interactome map of protein-protein interactions and a probabilistic neural network machine learning algorithm. The genes predictive of response to infliximab identified from baseline colon biopsy samples from two separate patient cohorts showed limited overlap in identity but significant overlap on the Human Interactome and were predictive of response to infliximab in a cross-cohort validation. The patients in these two cohorts are all diagnosed with UC, and as such, differences in the biology between these individuals may not manifest in large fold-changes in gene expression. These subtle differences in transcript levels may be overlooked in other differential gene expression methods. However, this study identified small but significant changes in gene expression that may lead to different treatment outcomes.

There is an interaction between genetic, immune, and environmental factors that is evident in the mucosa gene expression profiles of IBD patients compared to healthy controls and in the genetic risk alleles associated with an increased risk of IBD. Jostins L, et al. Nature. 491 (7422): 119-24 (2012), which is incorporated herein by reference for all purposes. The topological and biological properties of the infliximab response module on the Human Interactome suggests that it is possible to determine a molecular signature for inadequate response to TNFi therapies in patients with UC. TNFi therapies have demonstrated efficacy in the treatment of moderate to severe IBD. However, response rates vary, and initially 40-60% of patients fail to achieve remission with their initial treatment, dose escalation is needed in 23-46% of patients after 12 weeks of treatment and up to 50% of patients who responded initially will have a secondary loss of response after 12 months of therapy. Ford A C, et al. Am J Gastroenterol. 106 (4): 644-59, quiz 60 (2011); Sandborn W J, et al. Gastroenterology. 142 (2): 257-65 e1-3 (2012); Zampeli E, et al. World J Gastrointest Pathophysiol. 5(3):293-303 (2014); Rutgeerts P, et al. N Engl J Med. 353 (23): 2462-76 (2005); Roda G, et al. Clin Transl Gastroenterol. 7: e135 (2016); Fausel R, Afzali A. Ther Clin Risk Manag. 11:63-73 (2015); Fine S, et al. Gastroenterol Hepatol (N Y). 15 (12): 656-65 (2019), which are incorporated herein by reference for all purposes.

Given the need to rapidly manage disease flares and avoid surgery, there is a critical need for a test that can predict which UC patients will benefit from TNFi therapy and who may consider alternative treatment options.

The two sets of response prediction genes described in this study have little overlap; however, they are unified in a common response module on the Human Interactome. This observation addresses one of the major concerns of biomarker irreproducibility; studies evaluating response prediction biomarkers rarely report the same genes. Many studies have reported prognostic indicators of response to TNFi therapies in UC. Arijs I, et al. Gut. 58 (12): 1612-9 (2009); Subramaniam K, etal. Intern Med J. 44 (5): 464-70 (2014); Garcia-Bosch O, etal. J Crohns Colitis. 7 (9): 717-22 (2013); Rismo R, et al. Scand J Gastroenterol. 47 (5): 538-47 (2012); Olsen T, et al. Cytokine. 46 (2): 222-7 (2009), which are incorporated herein by reference for all purposes.

A gene array study of UC mucosal biopsies identified gene panels predictive of response to infliximab with 95% sensitivity and 85% specificity. Arijs I, et al. Gut. 58 (12): 1612-9 (2009), which is incorporated herein by reference for all purposes. A prospective study determined the predictive value of pre-treatment mucosal T cell-related cytokine gene expression profiles in response to infliximab; expression of transcripts encoding IL-17A and IFN-γ were associated with remission after three infliximab infusions (OR=5.4, p=0.013 and OR=5.5, p=0.011, respectively). Rismo R, et al. Scand J Gastroenterol. 47 (5): 538-47 (2012), which is incorporated herein by reference for all purposes. These studies developed predictive models using machine learning approaches, calculating mean gene expression values, evaluating the highest fold changes in gene expression or taking a pathway-based approach to describe UC disease biology. None of these studies have been developed into a clinical test for care of UC patients. By mapping the response module, network analyses performed in this study enabled identification of biomarkers associated with a specific disease phenotype (inadequate response to infliximab), reduced the noise inherent to gene expression data and eliminated many false positives that can arise from small sample sizes and characteristics specific to demographics of a particular patient cohort.

This network-based approach evaluates protein interactions to select genes that reflect the biology of disease at the individual patient level. The cross-cohort validation of two predictive classifiers, developed using a response module found in the Human Interactome, suggests the existence of a molecular signature in baseline tissue samples that characterizes UC patients who will have an inadequate response to TNFi therapy. Further development of such a test may decrease the time to treatment response, thus allowing patients to get back to their normal, productive lives sooner while decreasing the burden on supportive family members. Furthermore, this method of biomarker discovery and classifier development can be applied across multiple disease areas with complex phenotypes and datasets containing molecular information. The platform described herein opens new, unprecedented opportunities to create new drug response modules, predict drug response in complex diseases, and achieve a goal of treating patients with the most effective treatment for their unique disease biology.

### Example 4: Predicting Response to Infliximab at Treatment Initiation-Ulcerative Colitis

### Overview

This cross-cohort study aimed to (1) determine a network-based molecular signature that predicts the likelihood of inadequate response to the tumor necrosis factor-a inhibitor (TNFi) therapy, infliximab, in ulcerative colitis (UC) patients and (2) address biomarker irreproducibility across different cohort studies. Whole-transcriptome microarray data were derived from biopsies of affected colon tissue from 2 cohorts of infliximab-treated UC patients (training N=24 and validation N=22). Response was defined as endoscopic and histologic healing at 4-6 weeks and 8 weeks, respectively. From the training cohort, genes with RNA expression that significantly correlated with clinical response outcomes were mapped onto the Human Interactome network map of protein-protein interactions to identify a largest connected component (LCC) of proteins indicative of infliximab response status in UC. Expression levels of transcripts within the LCC were fed into a probabilistic neural network model to generate a classifier that predicts inadequate response to infliximab. A classifier predictive of inadequate response to infliximab was generated and tested in a cross-cohort, blinded fashion; the AUC was 0.83 and inadequate response was predicted with a 100% positive predictive value and 64% sensitivity. Genes separately identified from the 2 cohorts that correlated with response to infliximab appeared distinct but mapped onto the same network region of the Human Interactome, reflecting a common underlying biology of response among UC patients. Cross-cohort validation of a classifier predictive of infliximab response status in UC patients indicates that a molecular signature of non-response to TNFi therapies is present in patients' baseline gene expression data. One goal of this study is to develop methods of treatment that predict which patients will have an inadequate response to targeted therapies and define new targets and pathways for therapeutic development.

### Introduction

Ulcerative colitis (UC) is a chronic, relapsing disease characterized by diffuse mucosal inflammation of the colon. Langan, Robert C., et al. “Ulcerative colitis: diagnosis and treatment.” American family physician 76.9 (2007): 1323-1330, which is incorporated herein by reference for all purposes. UC is part of a larger spectrum of chronic relapsing diseases of the intestinal tract classified as inflammatory bowel disease (IBD), which also includes Crohn's disease (CD). IBD is a growing health problem, and the estimated prevalence is 568 cases per 100,000 persons in the US and 827 cases per 100,000 persons in Europe. Kappelman, Michael D., et al. “Recent trends in the prevalence of Crohn's disease and ulcerative colitis in a commercially insured US population.” Digestive diseases and sciences 58.2 (2013): 519-525, which is incorporated herein by reference for all purposes. Approximately 20% of patients with UC present symptoms before age 20. Kelsen, Judith, and Robert N. Baldassano. “Inflammatory bowel disease: the difference between children and adults.” Inflammatory bowel diseases 14.suppl_2 (2008): S9-S11, which is incorporated herein by reference for all purposes. UC is diagnosed based on clinical presentation and endoscopic evidence of inflammation in the rectum that extends proximally into the colon. Clinical manifestations of active disease include bloody diarrhea, rectal urgency, abdominal pain, weight loss, and malaise. See Langan, Robert C., et al.

One treatment goal in UC is to induce remission and maintain a corticosteroid-free remission, which often requires use of a targeted therapy. Rubin, David T., et al. “ACG clinical guideline: ulcerative colitis in adults.” Official journal of the American College of Gastroenterology| ACG 114.3 (2019): 384-413, which is incorporated herein by reference for all purposes. Approved targeted therapies include anti-integrin α₄β₇ (e.g., vedolizumab), anti-interleukin-12 of 23 (e.g., ustekinumab), tumor necrosis factor inhibitor (TNFi; e.g., adalimumab, infliximab and golimumab) and Janus kinase inhibitor (JAKi; e.g., tofacitinib) therapies.

Selecting the right drug for individual patients from day 1 of treatment results in faster recovery, less pain, and improved quality of life, particularly in chronic progressive diseases such as UC. However, response to targeted therapies in UC are as low 20-50%. Clinical response in UC clinical trials is defined as a decline in Mayo score of ≥3 points and either a ≥30% relative decrease from baseline with at least a 1-point decrease in rectal bleeding or a rectal bleeding score of 0 or 1. See Rubin, David T., et al.; Schroeder, Kenneth W., William J. Tremaine, and Duane M. Ilstrup. “Coated oral 5-aminosalicylic acid therapy for mildly to moderately active ulcerative colitis.” New England Journal of Medicine 317.26 (1987): 1625-1629, which are incorporated herein by reference for all purposes. This definition of response is less stringent than treatment goals of clinical remission (Mayo score of 0-2) and mucosal healing (endoscopic score of 0-1). This highlights the urgent need to develop precision medicine tools for UC patients so that a therapy suitable to each patient's disease biology can be prescribed.

Analysis of the map of human disease biology called the Human Interactome can be used to interpret a patients' unique molecular signature in order to identify which therapy will work for which patient based on each individual's unique biology. Mellors, Theodore, et al. “Clinical validation of a blood-based predictive test for stratification of response to tumor necrosis factor inhibitor therapies in rheumatoid arthritis patients.” Network and Systems Medicine 3.1 (2020): 91-104, which is incorporated herein by reference for all purposes. Analysis of the topology and dynamics of the Human Interactome can reveal the underlying biological processes regulating many of the most common and difficult to treat diseases. This has resulted in the ability to discover novel targets, reprioritize known targets, and develop new biomarkers to predict drug response. See Mellors, Theodore, et al.; Gysi, D. M., et al. “Network medicine framework for identifying drug repurposing opportunities for COVID-19. ArXiv.” arXiv preprint arXiv:2004.07229 (2020), which are incorporated herein by reference for all purposes. Application of this technology is especially useful in human complex diseases such as autoimmune conditions like rheumatoid arthritis and UC. One goal is to develop methods of treatment to predict which patients will have an inadequate response to targeted therapies and define new drug targets and pathways for novel therapeutic development.

### Cohort Description

Training cohort, GSE14580: Twenty-four patients with active UC, refractory to corticosteroids or immunosuppression, underwent colonoscopy with biopsies from diseased colon within a week prior to the first intravenous infusion of 5 mg infliximab per kg body weight. Response to infliximab was defined as endoscopic and histologic healing at 4-6 weeks after first infliximab treatment (8 patients as responders and 16 patients as inadequate responders). This data also included 6 healthy controls. Total RNA was isolated from colonic mucosal biopsies, labelled, and hybridized to Affymetrix® Human Genome U133 Plus 2.0 Arrays. Arijs, Ingrid, et al. “Mucosal gene signatures to predict response to infliximab in patients with ulcerative colitis.” Gut 58.12 (2009): 1612-1619, which is incorporated herein by reference for all purposes.

Validation cohort, GSE12251: Twenty-two patients underwent colonoscopy with biopsy before infliximab treatment. Response to infliximab was defined as endoscopic and histologic healing at week 8 (12 patients as responders and 11 patients as inadequate responders). For 1 patient, data from 2 samples taken at different timepoints were available. RNA was isolated from pre-infliximab biopsies, labeled and hybridized to Affymetrix® Human Genome U133 Plus_2.0 Array. Arijs, Ingrid, et al. “Mucosal gene signatures to predict response to infliximab in patients with ulcerative colitis.” Gut 58.12 (2009): 1612-1619, which is incorporated herein by reference for all purposes.

This study was performed in accordance with the principles outlined in the Declaration of Helsinki.

### Microarray Analysis

The 2 datasets were downloaded using GEOquery R package. Before-treatment gene expression data were extracted by setting the visit time point to baseline. Probe IDs were converted to gene Entrez ID using the hgu133plus2.db database. The 2 datasets were merged by the common probe IDs. Batch effects were removed using ComBat from the sva R package. Leek, Jeffrey T., et al. “The sva package for removing batch effects and other unwanted variation in high-throughput experiments.” Bioinformatics 28.6 (2012): 882-883; Johnson, W. Evan, Cheng Li, and Ariel Rabinovic. “Adjusting batch effects in microarray expression data using empirical Bayes methods.” Biostatistics 8.1 (2007): 118-127, which are incorporated herein by reference for all purposes. To retain the biological differences between responders and inadequate responders, cohort-specific biomarkers were derived prior to applying ComBat.

### Human Interactome

The Human Interactome contains experimentally determined physical interactions between proteins. See Mellors, T., Withers, J. B., Ameli, A. et al.; Menche, Jörg, et al. “Uncovering disease-disease relationships through the incomplete interactome.” Science 347.6224 (2015): 1257601, which is incorporated herein by reference for all purposes. These interactions include, regulatory, metabolic, signaling, and binary interactions. The Human Interactome amalgamates data from more than 300 thousand interactions among 18 thousand proteins.

### Identification of Classifier Genes

For all genes, the Pearson correlation between the gene expression values and the response to treatment was determined. Santolini, Marc, et al. “A personalized, multiomics approach identifies genes involved in cardiac hypertrophy and heart failure.” NPJ systems biology and applications 4.1 (2018): 1-13, which is incorporated herein by reference for all purposes. The signal-to-noise ratio of each gene correlation was calculated by randomly shuffling the response outcome 100 times. Selected genes were then mapped onto the consolidated Human Interactome and the largest connected component (LCC) was determined.

### Classifier Design and Validation

Genes identified as discriminatory between responders and inadequate responders to infliximab that were in the LCC were used as features of a probabilistic neural network. González-Camacho, Juan Manuel, et al. “Genome-enabled prediction using probabilistic neural network classifiers.” BMC genomics 17.1 (2016): 1-16, which is incorporated herein by reference for all purposes. Classifier training was performed by implementing the R package pnn in Python. Chasset, P.-O. “PNN: Probabilistic neural network for the statistical language R,” which is incorporated herein by reference for all purposes. The classifier training included in-cohort validation using a leave-one-sample-out cross-validation where the classifier was blind to the response outcome of that left-out patient. The classifier was trained using the default smoothing parameter (σ=0.8). The classifier was validated on the validation cohort where the training cohort was used for feature selection and classifier training and the validation cohort was used for independent validation.

The classifier provided a probability for each patient reflecting whether or not that individual responded to infliximab treatment. The log-likelihood ratio of response and inadequate response probabilities were used to define a score for each patient and draw the receiver operating characteristic (ROC) curves by comparing the score to actual response outcomes. The area under the curve (AUC) determined the performance of the classifier. In cross-cohort assessment, the trained classifier was blind to the clinical outcomes of the validation cohort patients.

### Response Module Randomization

Response module was comprised of the largest connected component (LCC) formed by top genes when derived from both training and validation cohorts. None of the shared genes (STC1, PAPPA, SOD2 and HGF) between the 2 cohorts' top gene sets was a high degree node in the Human Interactome, that could have caused a high degree of perceived connectedness between the LCC genes from the 2 cohorts. Hence, nodes were randomly assigned to both cohorts uniformly at random.

### Identification of Gene Expression Features Predictive of Inadequate Response to Infliximab

To identify genes whose expression best distinguished responders from inadequate responders to infliximab, 2 publicly available UC patient gene expression datasets were downloaded for which the clinical outcomes data were available. See Arijs, I., Li, K., Toedter, G. et al. The training cohort data were analyzed to find genes with significant gene expression deviations between responders and inadequate responders (response prediction genes). See Santolini, M., Romay, M. C., Yukhtman, C. L. et al. Compared to differential methods that look for large fold-changes in gene expression between 2 groups, the methods described herein investigated small but significant changes (e.g., a high signal-to-noise ratio) between the two groups. Genes were ranked by decreasing value of signal-to-noise ratio and the top genes with the highest signal-to-noise ratio were selected as infliximab response discriminatory genes (FIG. 1, subpanels A-B).

### Refinement of Molecular Signature Genes Using the Human Interactome

The top genes from the training cohort for which expression values across patients were significantly correlated to clinical outcome after infliximab treatment were selected and mapped onto the Human Interactome network map of protein-protein interactions (FIG. 1, subpanels B-C). The cut-off of the top 123 probes was empirically determined, for example, as the minimum number of genes for the LCC size to plateau (FIG. 5). Although these genes were identified from gene expression data only, for example, proteins encoded by these genes formed several clusters with the largest one containing 12 proteins on the Human Interactome. The associated proteins to the set of 123 probes on the Human Interactome was significantly closer to each other than expected by chance (z-score of −2.07) (FIG. 1, subpanels B-C and Table 9). Absolute z-scores >1.6 have been associated with sub-networks of underlying disease biology. See Menche, J., Sharma, A., Kitsak, M. et al.; Sharma, A., Menche, J., Huang, C. C. et al.; Sharma, Amitabh, et al. “A disease module in the interactome explains disease heterogeneity, drug response and captures novel pathways and genes in asthma.” Human molecular genetics 24.11 (2015): 3005-3020; Barabási, Albert-László, Natali Gulbahce, and Joseph Loscalzo. “Network medicine: a network-based approach to human disease.” Nature reviews genetics 12.1 (2011): 56-68; Ghiassian, Susan Dina, et al. “Endophenotype network models: common core of complex diseases.” Scientific reports 6.1 (2016): 1-13, which are incorporated herein by reference for all purposes.

TABLE 9

AMIGO2
CEBPB
CXCL1
CXCL2
CXCL6
DRAM1
IGFBP5
MAP3K20
MEIS1
MMP12
MS4A7
NR3C1

### Classifier Training and Blinded Cross-Cohort Validation

The LCC genes from the training cohort were used to train a probabilistic neural network; (Specht, Donald F. “Generation of polynomial discriminant functions for pattern recognition.” IEEE Transactions on Electronic Computers 3 (1967): 308-3 19; Specht, Donald F. “Probabilistic neural networks and the polynomial adaline as complementary techniques for classification.” IEEE Transactions on Neural Networks 1.1 (1990): 111-121, which are incorporated herein by reference for all purposes) an optimum pattern classifier that minimizes the risk of incorrectly classifying an object with high efficiency. See Gonzalez-Camacho, J. M., Crossa, J., Perez-Rodriguez, P., Ornella, L. & Gianola, D. The probabilistic neural networks were trained using the LCC genes and patient data to teach the predictive classifier the appropriate patient outcome (e.g., response or inadequate response to infliximab) for each input (e.g., gene expression levels of LCC genes).

Blinded, independent cross-cohort validation assessed the performance of the predictive classifier. In this analysis, the classifier that was trained on the known data and outcomes from the training cohort was used to predict the outcomes on the validation cohort, ultimately testing the ability of the classifier to accurately predict inadequate response to infliximab in an unseen patient population. The classifier predicted probabilities were converted to a continuous classifier prediction score using log-likelihood ratio. ROC curves, which plot the rate of false positives versus the rate of true positives, were used to assess cross-cohort performance (FIG. 2, subpanel A). An AUC of 0.83 was observed for the classifier predicting inadequate response to infliximab among validation cohort patients. The distribution of classifier prediction scores within the validation cohort showed a significant difference between the classifier prediction scores for responders and inadequate responders (P-value=0.004) (FIG. 2, subpanel B). Additionally, the cross-cohort positive predictive value (PPV) and sensitivity were estimated (FIG. 2, subpanel C), which are metrics that describe the accuracy of the inadequate response predictions. Ata 100% PPV, the classifier had a sensitivity of 64%.

### Baseline Gene Expression Profiles of Responders More Closely Resemble Healthy Controls

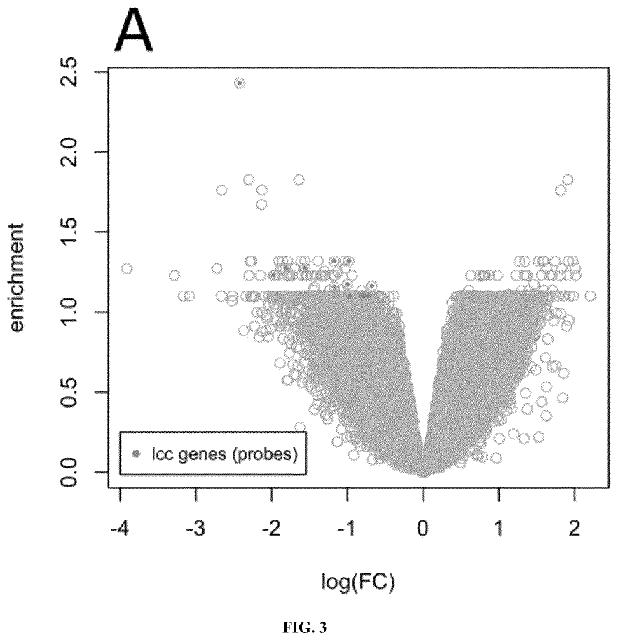

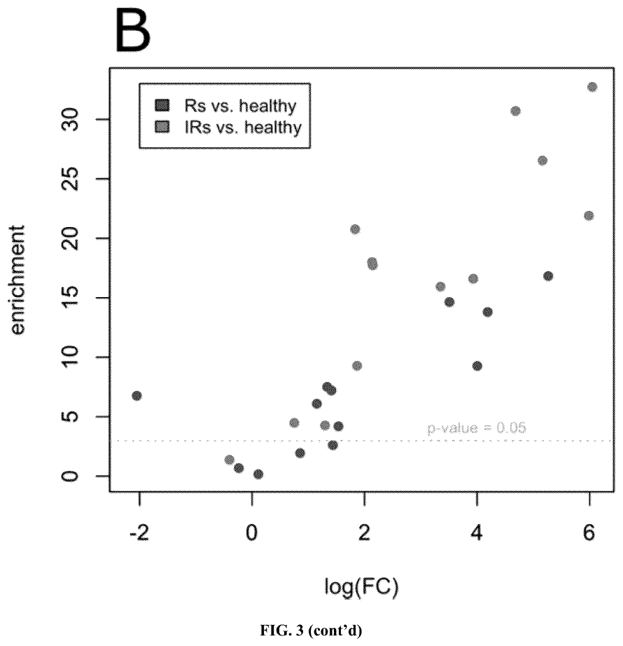

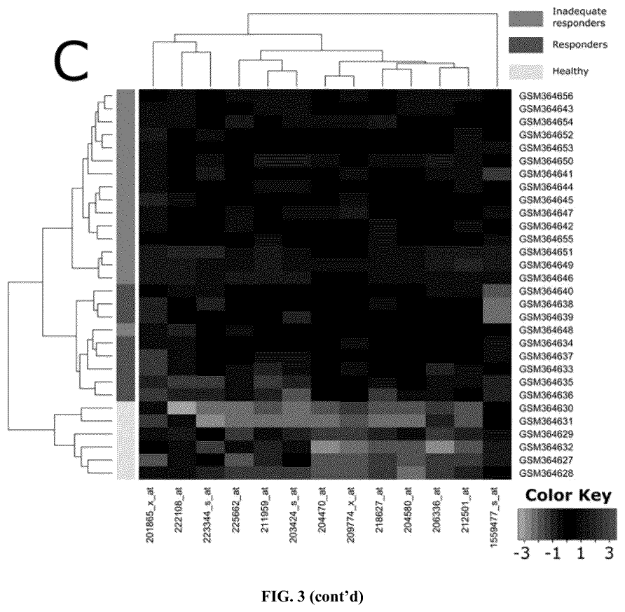

To further evaluate the 12-LCC classifier genes, the expression was compared between responders and inadequate responders. In general, the changes in gene expression between these 2 patient groups were small and no gene reached a threshold that indicated a significant differential expression (P-value≤0.05 and enrichment of −log(0.05)≥2.99) (FIG. 3, subpanel A). However, the expression of the 12-LCC classifier genes tended toward higher enrichment scores. One interpretation was that the genes with the greatest fold-change were not necessarily the most relevant to treatment response as they could potentially be downstream of master regulators or be altered by indirect/secondary consequences of disease-relevant signaling processes.

Next, the expression of the 12-LCC classifier genes was compared to that of healthy controls, comparing the fold changes relative to responders or to inadequate responders. The patients who were inadequate responders showed the largest divergence in gene expression pattern from that of healthy controls (FIG. 3, subpanel B). Unsupervised hierarchical clustering analysis showed that the baseline expression profiles of the patients who responded to infliximab more closely resembled the expression pattern of healthy controls than did the inadequate responders (FIG. 3, subpanel C).

### The UC Infliximab Response Module is a Sub-Network on the Human Interactome

The Human Interactome can serve as a blueprint to better understand the interconnectivity and underlying biology of the inadequate response prediction genes. Thus, the top 200 probes with the highest signal-to-noise ratio between responders and inadequate responders among the validation cohort data were also determined. When the 200 top probes from the training and validation cohorts were mapped simultaneously onto Human Interactome, the genes were not randomly scattered on the network, but instead significantly interacted with each other (z-score, absolute value of 7.68) forming a common response module LCC (FIG. 4, subpanel A) that was significantly larger than the random expectation (139 genes; z-score of 2.09). To account for genes that were shared between the 2 cohort gene lists, a careful randomization was made to estimate the significance of interconnectivity. Three proteins in the response module LCC (GK, FFAR2, CEBPB) are direct interaction partners of TNF-α, the protein target of infliximab. Several proteins in the response module LCC were orphan genes that were not previously part of LCCs of the individual cohorts (e.g., STC1 and IL7R) yet were integrated into this response module LCC (FIG. 4, subpanel A). These results shows that even though the response discriminatory genes identified from each cohort were apparently distinct with minimal overlap, their protein products tended to interact significantly on the network, reflecting the existence of an underlying disease biology subnetwork, or response module, that defined a molecular signature of non-response to infliximab in UC patients.

### Discussion

This study describes a predictive classifier developed using knowledge from the Human Interactome map of protein-protein interactions and a probabilistic neural network machine learning algorithm. The genes correlating with response to infliximab identified from baseline colon biopsy samples were predictive of inadequate response to infliximab in a cross-cohort validation. The patients in this study were all diagnosed with UC, and as such, differences in the biology between these individuals may not manifest in large fold-changes in gene expression. These subtle differences in transcript levels may be overlooked in other differential gene expression methods in favor of genes with a greater observed fold-change between the responder and inadequate responder groups. However, methods described herein identified small but significant changes in gene expression that may contribute to different treatment outcomes. The network-based method to discover biomarkers described in this study ensured that the differentially expressed genes in the classifier were significantly connected to the subnetwork of ulcerative colitis disease biology. This reduces the large number of differentially expressed genes to those most relevant to the biology of treatment response.

When response discriminatory genes identified separately from the training and validation cohorts were compared, the gene sets showed limited overlap in identity but significant overlap on the Human Interactome. Thus, they are unified in a common response module on the Human Interactome. This observation addresses one of the major concerns of biomarker irreproducibility; studies evaluating response prediction biomarkers rarely report the same genes. Many studies have reported prognostic indicators of response to TNFi therapies in UC. See Arijs, I., Li, K., Toedter, G. et al; Subramaniam, Kavitha, et al. “Early predictors of colectomy and long-term maintenance of remission in ulcerative colitis patients treated using anti-tumour necrosis factor therapy.” Internal medicine journal 44.5 (2014): 464-470; Garcia-Bosch, Orlando, et al. “Observational study on the efficacy of adalimumab for the treatment of ulcerative colitis and predictors of outcome.” Journal of Crohn's and Colitis 7.9 (2013): 717-722; Rismo, Renathe, et al. “Mucosal cytokine gene expression profiles as biomarkers of response to infliximab in ulcerative colitis.” Scandinavian journal of gastroenterology 47.5 (2012): 538-547; Olsen, Trine, etal. “TNF-alpha gene expression in colorectal mucosa as a predictor of remission after induction therapy with infliximab in ulcerative colitis.” Cytokine 46.2 (2009): 222-227, which are incorporated herein by reference for all purposes. A gene array study of UC mucosal biopsies identified gene panels predictive of response to infliximab with 95% sensitivity and 85% specificity. See Gysi, D. M., Do Valle, I., Zitnik, M. et al. A prospective study determined the predictive value of pre-treatment mucosal T cell-related cytokine gene expression profiles in response to infliximab; (See Rismo, R., Olsen, T., Cui, G. et al.) expression of transcripts encoding IL-17A and IFN-γ were associated with remission after 3 infliximab infusions (odds ratio or 5.4, P=0.013 and 5.5, P=0.011, respectively). These studies developed predictive models using machine learning approaches, calculatingmean gene expression values, evaluatingthe highest fold changes in gene expression or taking a pathway-based approach to describe UC disease biology. None of these studies have been developed into a clinical method of treatment for care of UC patients. By mapping the response module, network analyses according to the methods describe herein enabled identification of biomarkers associated with a specific disease phenotype (inadequate response to infliximab), reduced the noise inherent to gene expression data and eliminated many false positives that can arise from small sample sizes and characteristics specific to demographics of a particular patient cohort. Future analyses and larger cohort studies will explore the use of genes in the aggregated response module to train and validate a TNFi response classifier.

The proteins encoded by the classifier genes of Table 9 are implicated in many biological processes including epithelial cell proliferation, response to reactive oxygen species, regulation of apoptotic signaling and cellular responses to lipid metabolism. Bioactive lipid mediators, including prostaglandins, regulate chronic inflammation through cell differentiation and activation, protect against acute epithelial barrier damage and facilitate tissue regeneration. Crittenden, Siobhan, et al. “Prostaglandin E2 promotes intestinal inflammation via inhibiting microbiota-dependent regulatory T cells.” Science advances 7.7 (2021): eabd7954; Yao, Chengcan, and Shuh Narumiya. “Prostaglandin-cytokine crosstalk in chronic inflammation.” British journal of pharmacology 176.3 (2019): 337-354; Kabashima, Kenji, et al. “The prostaglandin receptor EP4 suppresses colitis, mucosal damage and CD4 cell activation in the gut.” The Journal of clinical investigation 109.7 (2002): 883-893; Roulis, Manolis, et al. “Intestinal myofibroblast-specific Tpl2-Cox-2-PGE2 pathway links innate sensing to epithelial homeostasis.” Proceedings of the National Academy of Sciences 111.43 (2014): E4658-E4667; Yao, Chengcan, etal. “Prostaglandin E2-EP4 signaling promotes immune inflammation through THI cell differentiation and TH17 cell expansion.” Nature medicine 15.6 (2009): 633-640, which are incorporated herein by reference for all purposes TNF-α protein production is elevated in UC patients. MacDonald, Thomas T., et al. “Tumour necrosis factor-alpha and interferon-gamma production measured at the single cell level in normal and inflamed human intestine.” Clinical & Experimental Immunology 81.2 (1990): 301-305; Murch, S. H., et al. “Serum concentrations of tumour necrosis factor alpha in childhood chronic inflammatory bowel disease.” Gut 32.8 (1991): 913-917; Komatsu, Momoko, et al. “Tumor necrosis factor-α in serum of patients with inflammatory bowel disease as measured by a highly sensitive immuno-PCR.” Clinical chemistry 47.7 (2001): 1297-1301; Martinez-Borra, Jesús, et al. “High serum tumor necrosis factor-α levels are associated with lack of response to infliximab in fistulizing Crohn's disease.” The American journal of gastroenterology 97.9 (2002): 2350-2356; Yarur, Andres J., etal. “The association of tissue anti-TNF drug levels with serological and endoscopic disease activity in inflammatory bowel disease: the ATLAS study.” Gut 65.2 (2016): 249-255; Braegger, Christian P., et al. “Tumour necrosis factor alpha in stool as a marker of intestinal inflammation.” The Lancet 339.8785 (1992): 89-91, which are incorporated herein by reference for all purposes. TNF stimulation induces COX-2 expression in innate immune cells, initiating proinflammatory responses by converting arachidonic acid into prostaglandins and inducing production of other cytokines and chemokines. Chen, Chu. “COX-2's new role in inflammation.” Nature chemical biology 6.6 (2010): 401-402, which is incorporated herein by reference for all purposes. Inclusion of genes encoding proteins involved in epithelial cell proliferation and cell apoptosis indicatesa role for barrier function in determining TNFi inadequate response. Ye, Dongmei, Iris Ma, and Thomas Y. Ma. “Molecular mechanism of tumor necrosis factor-a modulation of intestinal epithelial tight junction barrier.” American Journal of Physiology-Gastrointestinal and Liver Physiology 290.3 (2006): G496-G504, which is incorporated herein by reference for all purposes. A permeable barrier is formed by intestinal epithelial cells that normally restricts access to potential antigens in the intestinal lumen and epithelial regeneration is a critical aspect of mucosal healing and regeneration. Okamoto, Ryuichi, and Mamoru Watanabe. “Cellular and molecular mechanisms of the epithelial repair in IBD.” Digestive diseases and sciences 50.1 (2005): S34-S38; Okamoto, Ryuichi, and Mamoru Watanabe. “Cellular and molecular mechanisms of the epithelial repair in IBD.” Digestive diseases and sciences 50.1 (2005): S34-S38, which are incorporated herein by reference for all purposes. Disrupted barrier function contributes to the pathogenesis of inflammatory bowel diseases and TNF inhibitor treatment can improve intestinal barrier function in IBD. Noth, Rainer, et al. “Anti-TNF-α antibodies improve intestinal barrier function in Crohn's disease.” Journal of Crohn's and Colitis 6.4 (2012): 464-469; Bouma, Gerd, and Warren Strober. “The immunological and genetic basis of inflammatory bowel disease.” Nature Reviews Immunology 3.7 (2003): 521-533; Bouma, Gerd, and Warren Strober. “The immunological and genetic basis of inflammatory bowel disease.” Nature Reviews Immunology 3.7 (2003): 521-533, which are incorporated herein by reference for all purposes. Consistent with the complexity of TNF-α signaling, transcripts predictive of inadequate response to infliximab are similarly diverse.

There can be an interaction between genetic, immune, and environmental factors that is evident in the mucosa gene expression profiles of IBD patients compared to healthy controls and in the genetic risk alleles associated with an increased risk of IBD. Jostins, Luke, et al. “Host-microbe interactions have shaped the genetic architecture of inflammatory bowel disease.” Nature 491.7422 (2012): 119-124, which is incorporated herein by reference for all purposes. The topological and biological properties of the infliximab response module on the Human Interactome suggests that it is possible to determine a molecular signature for inadequate response to TNFi therapies in patients with UC. TNFi therapies have demonstrated efficacy in the treatment of moderate to severe IBD. However, response rates vary, and initially 40-60% of patients fail to achieve remission with their initial treatment, (Ford, Alexander C., et al. “Efficacy of biological therapies in inflammatory bowel disease: systematic review and meta-analysis.” Official journal of the American College of Gastroenterology| ACG 106.4 (2011): 644-659; Sandborn, William J., etal. “Adalimumab induces and maintains clinical remission in patients with moderate-to-severe ulcerative colitis.” Gastroenterology 142.2 (2012): 257-265; Zampeli, Evanthia, et al. “Predictors of response to anti-tumor necrosis factor therapy in ulcerative colitis.” World journal of gastrointestinal pathophysiology 5.3 (2014): 293, which are incorporated herein by reference for all purposes) dose escalation is needed in 23-46% of patients after 12 weeks of treatment (Roda, Giulia, et al. “Loss of response to anti-TNFs: definition, epidemiology, and management.” Clinical and translational gastroenterology 7.1 (2016): e135; Fausel, Rebecca, and Anita Afzali. “Biologics in the management of ulcerative colitis-comparative safety and efficacy of TNF-α antagonists.” Therapeutics and clinical risk management 11 (2015): 63, which ARE incorporated herein by reference for all purposes) and up to 50% of patients who respondedinitially will have a secondary loss of response after 12 months of therapy. See Roda, G., Jharap, B., Neeraj, N. & Colombel, J. F.; Fine, Sean, Kostantinos Papamichael, and Adam S. Cheifetz. “Etiology and Management of Lack or Loss of Response to Anti-Tumor Necrosis Factor Therapy in Patients With Inflammatory Bowel Disease.” Gastroenterology & Hepatology 15.12 (2019): 656, which are incorporated herein by reference for all purposes. Furthermore, a multicenter, retrospective study reported that 55.6% of UC patients who were primary non-responders to infliximab underwent colectomy within 3.2 years, a surgery that costs an estimated $91,767. Papamichael, Konstantinos, et al. “Long-term outcome of patients with ulcerative colitis and primary non-response to infliximab.” Journal of Crohn's and Colitis 10.9 (2016): 1015-1023; Papamichael, Konstantinos, et al. “Long-term outcome of patients with ulcerative colitis and primary non-response to infliximab.” Journal of Crohn's and Colitis 10.9 (2016): 1015-1023, which are incorporated herein by reference for all purposes. Given the need to rapidly manage disease flares and avoid surgery, there is a critical need for a method of treatment that can predict which UC patients will benefit from TNFi therapy and who should consider alternative treatment options.

This network-based approach described herein evaluates protein interactions to select genes that reflect the biology of disease at the individual patient level. The cross-cohort validation of the predictive classifier developed using a response module found in the Human Interactome, suggests the existence of a molecular signature in baseline tissue samples that characterizes UC patients who will have an inadequate response to TNFi therapy. Further development of such a method of treatment may decrease the time to treatment response, thus allowing patients to get back to their normal, productive lives sooner while decreasing the burden on supportive family members. Furthermore, this method of biomarker discovery and classifier development can be applied across multiple disease areas with complex phenotypes and datasets containing molecular information. The platform described herein opens new, unprecedented opportunities to create new drug response modules, predict drug response in complex diseases, and achieve a goal of treating patients with the most effective treatment for their unique disease biology.

The foregoing has been a description of certain non-limiting embodiments of the subject matter described within. Accordingly, it is to be understood that the embodiments described in this specification are merely illustrative of the subject matter reported within. Reference to details of the illustrated embodiments is not intended to limit the scope of the claims, which themselves recite those features regarded as essential.

It is contemplated that systems and methods of the claimed subject matter encompass variations and adaptations developed using information from the embodiments described within. Adaptation, modification, or both, of the systems and methods described within may occur.

Throughout the description, where systems are described as having, including, or comprising specific components, or where methods are described as having, including, or comprising specific steps, it is contemplated that, additionally, there are systems encompassed by the present subject matter that consist essentially of, or consist of, the recited components, and that there are methods encompassed by the present subject matter that consist essentially of, or consist of, the recited processing steps.

It should be understood that the order of steps or order for performing certain action is immaterial so long as any embodiment of the subject matter described within remains operable. Moreover, two or more steps or actions may be conducted simultaneously.

While preferred embodiments of the present disclosure have been shown and described herein, such embodiments are provided by way of example only. Numerous variations, changes, and substitutions may occur without departing from the present disclosure. It should be understood that various alternatives to the embodiments of the disclosure described herein may be employed in practicing the present disclosure. It is intended that the following claims define the scope of the present disclosure and that methods and structures within the scope of these claims and their equivalents be covered thereby.

1.-78. (canceled)

79. A method of treating a subject suffering from ulcerative colitis (UC), the method comprising:
administering to the subject an anti-TNF therapy,
wherein the subject has been predicted to be responsive to the anti-TNF therapy based at least in part on a trained machine learning classifier that distinguishes between responsive subjects and non-responsive subjects who have received the anti-TNF therapy,
wherein the trained machine learning classifier distinguishes between the responsive subjects and the non-responsive subjects, based at least in part on analyzing an expression level in the subject of a set of genes.

80. The method of claim 79, wherein the trained machine learning classifier further analyzes:
presence of one or more single nucleotide polymorphisms (SNPs) in a sequence of one or more genes that is expressed in the subject; or
presence of one or more clinical characteristics of the subject.

81. The method of claim 80, wherein the one or more clinical characteristics of the subject comprise body-mass index (BMI), gender, age, race, previous anti-TNF therapy treatment, disease duration of ulcerative colitis (UC), C-reactive protein level, or treatment response rate to the anti-TNF therapy.

82. The method of claim 79, wherein the trained machine learning classifier predicts the subject to be responsive to the anti-TNF therapy using a non-linear relationship between (i) an expression level of one or more genes identified in the subject and (ii) responsiveness or non-responsiveness to the anti-TNF therapy.

83. The method of claim 79, wherein the trained machine learning classifier is trained using expression levels of a set of genes in (i) a first set of subjects with ulcerative colitis (UC) who were responsive to the anti-TNF therapy and (ii) a second set of subjects with ulcerative colitis (UC) who were non-responsive to the anti-TNF therapy.

84. The method of claim 79, wherein the trained machine learning classifier is validated by validating the classifier on a second independent cohort of subjects who have received the anti-TNF therapy and have been determined as either responding to the anti-TNF therapy or not responding to the anti-TNF therapy.

85. The method of claim 84, wherein validating the classifier further comprises using the classifier to predict a probability of response of at least one of the second independent cohort of subjects.

86. The method of claim 79, wherein the trained machine learning classifier comprises a neural network or a random forest.

87. The method of claim 79, wherein the trained machine learning classifier predicts that subjects within a population are responsive or non-responsive to the anti-TNF therapy with a true negative rate (TNR) of at least about 60%.

88. The method of claim 79, wherein the trained machine learning classifier predicts that subjects within a population are responsive or non-responsive to the anti-TNF therapy with a negative predictive value (NPV) of at least about 85%.

89. The method of claim 79, wherein the trained machine learning classifier predicts that subjects within a population are responsive or non-responsive to the anti-TNF therapy with an area under the curve (AUC) of at least about 70%.

90. The method of claim 79, wherein the trained machine learning classifier predicts that subjects within a population are responsive or non-responsive to the anti-TNF therapy with an accuracy of at least about 90%.

91. The method of claim 79, wherein the set of genes comprises ABCC5, ABHD12, ABL2, ABTB1, ADAMTS12, AJUBA, AMIGO2, AP2A2, AQP9, ATG4B, BAD, BAG5, BCL2A1, BCL6, BMP1, C16orf58, C5AR1, CANX, CCNB1, CD82, CDCA7L, CEBPB, CFLAR, CHEK1, CHN2, CHST10, CHTOP, CLEC4E, CREB5, CSF3R, CXCL1, CXCL11, CXCL2, CXCL6, CXCL8, CXCR1, CYP4F3, DRAM1, DUSP1, DYRK1A, ECH1, ECSIT, EPS15L1, FAM86C1, FCGR1B, FCGR1CP, FCGR3B, FCGR3B|FCGR3A, FCN1, FFAR2, FGF2, FGR, FPR1, FPR2, FST, GABARAPL1, GALNT15, GK, GK3P, GNAI1, H2BC3, HGF, IDH3B, IFIT2, IFIT3, IGFBP5, IL13RA2, IL1B, IL7R, INHBA, IRAK2, KIFC1, KLHL12, LARP4B, LATS2, LIMS1, LRRC8C, MAP3K20, MASP1, MEFV, MEIS1, MMP12, MNDA, MS4A7, NAAA, NBN, NFE2L1, NFIL3, NINJ1, NR3C1, NUP88, OTX1, PAPPA, PAX5, PI15, PLAU, PLEK, PLG, PPM1A, PTGS2, PTK2B, RGS5, RHBDD1, RIPK2, RNF144B, RPIA, RUVBL2, S100A9, SET, SIAH2, SLC22A4, SLC25A29, SLC35G2, SLC7A8, SMC2, SNCA, SNX29, SOD2, SPIRE2, SPPL3, SSRP1, STC1, SUPV3L1, TAL1, TARDBP, TLR2, TLR4, TMEM97, TNC, TNFAIP6, TOLLIP, TOR1AIP1, TRAF1, TRDV2, TREM1, TRIM8, TSN, USP54, or ZNF57.

92. The method of claim 91, wherein the set of genes comprises AMIGO2, CEBPB, CXCL1, CXCL2, CXCL6, DRAM1, IGFBP5, MAP3K20, MEIS1, MMP12, MS4A7, or NR3C1.

93. The method of claim 79, wherein the anti-TNF therapy comprises infliximab, adalimumab, etanercept, certolizumab pegol, golimumab, or a biosimilar thereof.

94. The method of claim 79, wherein an alternative to the anti-TNF therapy is administered when the trained machine learning classifier predicts the subject to be non-responsive to the anti-TNF therapy.

95. The method of claim 94, wherein the alternative to the anti-TNF therapy comprises rituximab, sarilumab, tofacitinib citrate, leflunomide, vedolizumab, tocilizumab, anakinra, abatacept or a biosimilar thereof.

96. The method of claim 79, wherein the expression level is obtained by microarray, RNA sequencing, real-time quantitative reverse transcription PCR (qRT-PCR), bead array, or ELISA.

97. The method of claim 79, further comprising analyzing a biological sample of the subject by microarray, RNA sequencing, real-time quantitative reverse transcription PCR (qRT-PCR), bead array, or ELISA.

98. The method of claim 97, further comprising obtaining expression levels of the one or more genes from the biological sample.
